(19) United States
(12) Patent Application Publication
(10) Pub. No.: US 2025/0243238 A1
(43) Pub. Date: Jul. 31, 2025

(54) TETRAZINE AMINO ACIDS AND METHODS FOR THEIR PRODUCTION AND USE

(71) Applicant: Oregon State University, Corvallis, OR (US)

(72) Inventors: Ryan A. Mehl, Corvallis, OR (US); Yogesh Gangarde, Corvallis, OR (US); Subhashis Jana, Corvallis, OR (US); Alex J. Eddins, Corvallis, OR (US)

(73) Assignee: Oregon State University, Corvallis, OR (US)

(21) Appl. No.: 19/043,285

(22) Filed: Jan. 31, 2025

Related U.S. Application Data
(60) Provisional application No. 63/627,687, filed on Jan. 31, 2024.
(60) Provisional application No. 63/627,674, filed on Jan. 31, 2024.
(60) Provisional application No. 63/627,665, filed on Jan. 31, 2024.
(60) Provisional application No. 63/627,625, filed on Jan. 31, 2024.

(51) Int. Cl. C07K 1/00; C07D 401/04; C07K 1/13; C12N 9/00; C12N 15/113

(52) U.S. Cl. CPC C07K 1/006; C07D 401/04; C07K 1/13; C12N 9/93; C12N 15/113; C12Y 601/01

(57) ABSTRACT

Tetrazine non-canonical amino acids, methods for making the tetrazine non-canonical amino acids, methods for incorporating the tetrazine non-canonical amino acids into proteins and polypeptides, post-translationally modified proteins and polypeptides in which the tetrazine non-canonical amino acids have been incorporated, and kits for incorporating the tetrazine non-canonical amino acids into proteins and polypeptides.

### CROSS-REFERENCE TO RELATED APPLICATIONS

This application claims the benefit of Application No. 63/627,687, filed Jan. 31, 2024, Application No. 63/627,674, filed Jan. 31, 2024, Application No. 63/627,665, filed Jan. 31, 2024, and Application No. 63/627,625, filed Jan. 31, 2024, the disclosure of each of which is hereby expressly incorporated by reference in its entirety.

### STATEMENT OF GOVERNMENT LICENSE RIGHTS

This invention was made with government support under RM1-GM144227 awarded by National Institutes of Health, 1R01GM131168-01 awarded by National Institutes of Health, and NSF-2054824 awarded by National Science Foundation. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3014-P38WO Sequence Listing.xml. The XML file is 27,917 bytes; was created on Jan. 30, 2025; and is being submitted electronically via Patent Center with the filing of the specification.

### BACKGROUND

Encodable bioorthogonal ligations have become an indispensable and powerful tool for studying living process in cells because one of the two chemical needed functional groups can be encoded into specific locations on biopolymers and the second functionality can be used to deliver diverse small molecule probes, materials, or cellular components. Encodable bioorthogonal ligation systems are challenging systems to develop because three key comprising elements; the bioorthogonal chemistry, the functional group encoding and the delivery of the label into the cell, are interconnected and each need to be optimized for the labeling tool to work effectively. For this reason, many catalyst-free bioorthogonal reactions (Staudinger ligation, strain promoted azide-alkyne cycloaddition, oxime ligation, photo induced tetrazole-alkene ligation, and strain promoted tetrazine-alkene cycloaddition) have been explore for ability in encodable bioorthogonal ligations. Bioorthogonal ligations that used small functional groups were critical in launching the field because the traditional functional group encoding step relied on the natural cellular enzymes which could tolerate only small deviations in structure from natural substrates. Include a sentence here about tetrazine encoding.

Genetic code expansion (GCE) can now be engineered to encode a wide variety of bioorthogonal functional groups. In GCE, a reactive functional group is converted into a noncanonical amino acid (ncAA) which then enters protein translation using an evolved tRNA/RS pair specific for the ncAA. Using this approach, many bioorthogonal functional groups components have been encoded site-specifically into proteins and evaluated for labeling in cell and on cell surfaces. The extensive work on developing and using labeling reactions in live cells make clear that high biomolecular rate constants are critical to advance the ability to monitor biological process in live cells, in real time. Exceptionally high reaction rates are the only way to access the uncatalyzed labeling of low intracellular protein concentrations with low label concentrations in short time frames. Unfortunately, very few encodable bioorthogonal functional groups can be structurally altered to increase bioorthogonal reaction rates.

Recently, the 1,2,4,5-tetrazine-alkene cycloaddition reaction (tetrazine reaction), also known as the strain-promoted inverse electron demand Diels-Alder (IEDDA) reaction has been explored extensively because of its extraordinarily fast bioorthogonal reaction rates. In addition to exceptionally high reaction rates for biorthogonal reactions, the tetrazine reaction also has an usually broad functional reaction rate range (1 to 10⁶ M⁻¹s⁻¹). The broad reaction rate range results from the electron donating or withdrawing substituent effects on the tetrazine diene and the variety of ring strain imposed on the alkene dienophile. There have been numerous studies that explore the chemical range of functional groups, theoretical limits, and their use in bioorthogonal applications. Thus far many ncAA comprising of the ring strained alkene dienophile have been encoded into proteins to determine the reaction limits of genetically encoded tetrazine reactions but very few tetrazine ncAA structures have been explored.

The methanogenic archaea derived pyrrolysyl-tRNA/tRNA-synthetase systems (PylRS/tRNA^{Pyl}) which can function efficiently as an orthogonal GCE tool across most domains of life has been used extensively to encode strained alkenes attached as a lysine derivative. The ease at which diverse large hydrophobic rings can be encoded as lysine derivatives with the PylRS/tRNA^{Pyl} has allowed on protein and in cell reactivity and stability assessment of strained alkenes having different ring size, attachment chemistry, and levels of ring size. Obtaining reaction rates with 10³ M⁻¹s⁻¹ is possible by encoding strain alkenes without losing significant amino acid stability. Unfortunately, once reaction rates are pushed above 10⁴ M⁻¹s⁻¹ using the more reactive transcyclooctene (TCO) ncAAs a significant portion of the ncAA protein is no longer reactive to Tet-labels due to isomerization of the trans bond from cellular conditions.

As an alternative approach to maximize encoded bioorthogonal reaction rates, encoding Tet ncAAs and reacting with sTCO labels has been reported. The synthesis and encoding of first-generation Tet ncAAs using an Mj tRNA synthetase MjRS/tRNA^{Mj} pair was demonstrated in 2012. A required part of the synthesis the amine linkage in first-generation Tet ncAAs resulted in a slow tetrazine reaction (10² M⁻¹s⁻¹) and compromised the stability of the final labeled protein due to low levels of amine elimination. Newer synthetic routes to make tetrazines provided second- and third-generation Tet ncAAs, which were encoded with the Mj-RS/tRNA^{Mj} and PylRS/tRNA^{Pyl} pairs. These newer Tet ncAA-containing proteins were able to achieve reaction rates of 2 to 8×10⁴ M⁻¹s⁻¹ in live E. coli and mammalian cells while maintaining complete reactivity and product stability.

Despite the advancement in the development of Tet ncAAs for encoding into proteins for rapid bioorthogonal labeling, a need exists for improved Tet ncAAs. The present disclosure seeks to fulfill this need and provide further related advantages.

### SUMMARY

In one aspect, the disclosure provides a tetrazine amino acid having formula (I):

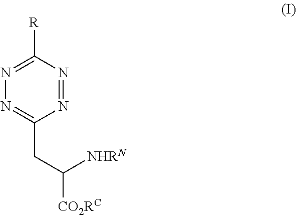

or a stereoisomer or salt thereof, wherein
R is selected from the group consisting of:
(a) a phenyl group substituted with a group selected from o-C1-C4 alkyl, m-C1-C4 alkyl, o-C1-C3 alkoxy, m-C1-C3 alkoxy, o-cyano, m-cyano, o-nitro, m-nitro, o- or m-primary amino (—NH₂), secondary amino (—NHR^{x}), or tertiary amino (—NHR^{x}R^{y}) (wherein R^{x} and R^{y} are independently C1-C6 alkyl), o-fluoro, m-fluoro, 3,5-difluoro, 3,4,5-trifluoro, o-trifluoromethyl, m-trifluoromethyl, and o-, m-, or p-C(═O)R^{z} (wherein R^{z} a counterion, hydrogen, or C1-C6 alkyl),
(b) a substituted or an unsubstituted heteroaryl group,
(c) a substituted or an unsubstituted heterocyclyl group,
(d) an amino C1-C6 alkyl group,
(e) a thio C1-C6 alkyl group,
(f) a carboxylate group,
(g) a sulfonate group, and
(h) an amide group;
R^{C} is hydrogen, a counter ion, or a carboxyl protecting group; and
R^{N} is hydrogen or an amine protecting group.

In certain embodiments, the C1-C6 alkyl group is selected from the group consisting of methyl, ethyl, n-propyl, i-propyl, n-butyl, s-butyl, t-butyl, n-pentyl, and n-hexyl.

In certain embodiments, the phenyl group is substituted with a group selected from the group consisting of 3-methyl, 2-methoxy, 3-methoxy, 2-cyano, 3-cyano, 2-nitro, 3-nitro, 2-fluoro, 3-fluoro, 3,5-difluoro, 3,4,5-trifluoro, 2-hydroxy, 3-hydroxy, 2-amino, 3-amino, 2-acetyl, 2-trifluoromethyl, and 3-trifluoromethyl.

In certain embodiments, the heteroaryl group is a pyridyl group, a furanyl group, a thiophenyl group, or an oxazolyl group.

In certain embodiments, the heterocyclyl group is a pyrrolidinyl group, a piperadinyl group, a piperazinyl group, a tetrahydrofuranyl group, a tetrahydropyranyl, a tetrahydrothiophenyl group, an oxazolydinyl group, or a dihydropyran group.

In another aspect, the disclosure provides a method for making a protein or a polypeptide of interest, comprising incorporating a tetrazine amino acid or a stereoisomer or salt thereof, as described herein, into a protein or polypeptide.

In a related aspect, the disclosure provides a method for genetically encoding a protein or a polypeptide of interest, comprising incorporating a tetrazine amino acid or a stereoisomer or salt thereof, as described herein, into a protein or polypeptide by genetic encoding.

In further aspect, the disclosure provides protein or polypeptide, comprising at least one tetrazine amino acid residue, wherein the tetrazine amino acid residue is derived from a tetrazine amino acid or a stereoisomer or salt thereof, as described herein.

In a related aspect, the disclosure provides a protein or polypeptide, comprising at least one tetrazine amino acid residue, wherein the tetrazine amino acid residue is incorporated into the protein or polypeptide by genetic encoding of the protein or polypeptide using a tetrazine amino acid or a stereoisomer or salt thereof, as described herein.

In another aspect, the disclosure provides a composition comprising a protein or polypeptide, wherein the protein or polypeptide comprises at least one tetrazine amino acid comprising a first reactive group and at least one post-translational modification, wherein the tetrazine amino acid residue is derived from a tetrazine amino acid or a stereoisomer or salt thereof, as described herein, and wherein the at least one post-translational modification comprises attachment of a molecule comprising a second reactive group by a [4+2] cycloaddition reaction to the at least one tetrazine amino acid comprising the first reactive group.

In a related aspect, the disclosure provides a composition comprising a protein or polypeptide, wherein the protein or polypeptide comprises at least one tetrazine amino acid comprising a first reactive group and at least one post-translational modification, wherein the tetrazine amino acid residue is derived from genetic encoding of the protein or polypeptide using a tetrazine amino acid or a stereoisomer or salt thereof, as described herein, and wherein the at least one post-translational modification comprises attachment of a molecule comprising a second reactive group by a [4+2] cycloaddition reaction to the at least one tetrazine amino acid comprising the first reactive group.

In a further aspect, the disclosure provides a kit for in cellulo production of a tetrazine-labeled protein or a tetrazine-labeled polypeptide, comprising (a) a tRNA; (b) an aminoacyl-tRNA synthetase; and (c) a tetrazine amino acid or a stereoisomer or salt thereof as described herein, wherein the tRNA and aminoacyl-tRNA synthetase are an orthogonal tRNA/orthogonal aminoacyl-tRNA pair effective for incorporating the compound into a protein or polypeptide to provide a tetrazine-labeled protein.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIG. 1 is an illustration depicting genetically encoded tetrazine amino acid that directs rapid site-specific in vivo bioorthogonal ligation with trans-cyclooctenes.

FIG. 2 is a schematic illustration of the preparation of representative tetrazine (Tz 3.0 or Tet-v3.0) non-canonical amino acids.

FIG. 3 is a schematic illustration of the preparation of representative tetrazine (Tet-v4.0) non-canonical amino acids.

FIGS. 4A-4J compare stop-flow kinetics measurements of representative tetrazine (Tet-v4.0) non-canonical amino acids in sTCO reaction relative to tetrazine (Tet-v2.0 methyl and Tet-v3.0 hydrogen) non-canonical amino acids.

FIG. 5 compares reaction kinetics of representative tetrazine (Tet-v4.0) non-canonical amino acids in sTCO reaction relative to a representative tetrazine (Tet-v3.0 hydrogen) non-canonical amino acid.

FIGS. 6A-6F compare qualitative analysis and mass characterization of sfGFP_tet (representative tetrazine (Tet-v3.0 hydrogen and butyl) non-canonical amino acids) with and without reaction with sTCO.

FIGS. 7A and 7B show expression (7A) and characterization (7B) of sfGFP expressed with selected aaRS and representative tetrazine (Tet-v4.0) non-canonical amino acids.

FIGS. 8A-8H compare mass spectrometric characterization of sfGFP with representative tetrazine (Tet-v4.0) non-canonical amino acids with and without reaction of sTCO.

FIGS. 9A-9F compare fluorescence kinetics measurements of representative sfGFP-tetrazine (Tet-v4.0) non-canonical amino acids in sTCO reaction relative to sfGFP-tetrazine (Tet-v3.0 hydrogen and butyl) non-canonical amino acids.

FIG. 10 is a schematic illustration of the preparation of a representative tetrazine (Tet-v3.0 hydrogen) amino acid.

FIGS. 11A and 11B are schematic illustrations of the preparation of intermediates useful in the preparation of representative tetrazine (Tet-v4.0) amino acids. FIG. 11A illustrates the synthesis of a methyl thiocarbohydrazide intermediate. FIG. 11B illustrates the synthesis of a thiomethyl tetrazine (Tet-v4.0 SMe) intermediate.

FIG. 12 is a schematic illustration of the preparation of representative tetrazine (Tet-v4.0) amino acids from the intermediate shown in FIG. 11B.

FIG. 13 is a schematic illustration of the preparation of a representative tetrazine amino acid (Tet-v4.0 aminobutyl) from the intermediate shown in FIG. 11B.

FIG. 14 is a schematic illustration of the preparation of representative tetrazine amino acids (Tet-v4.0) via hydrolysis of protected carboxylic acid and amino groups of tetrazine amino acid precursors.

FIG. 15 illustrates representative tetrazine amino acids (Tet-v4.0) prepared by the methods described herein.

FIG. 16 is a general synthetic scheme for the preparation of representative tetrazine amino acids (Tet-v4.0) and illustrates representative tetrazine amino acids (Tet-v4.0) prepared by the scheme.

FIG. 17 is a table that summarizes tetrazine amino acids (Tet-v4.0) and their reactivity, in media stability at physiological pH, and MaRS selection.

FIG. 18 is a table that summarizes MaRS hits and their active site assignments for representative tetrazine amino acids.

FIG. 19 illustrates the synthetic scheme for the preparation of representative Tet-v4.0 ncAAs having different donor and acceptor substituents R (synthetic yields in parentheses).

FIG. 20 illustrates the genetic incorporation of Tet-ncAAs. Permissivity and efficiency of evolved synthetases measured by florescence of expressed GFP-TAG150 in presence and absence of 0.3 mM Tet-v4.0 amino acids using D4 and E1 Tet-v4.0 synthetases in 25 mL AIM at 37° C. for 32 hrs. Asterisks (*) represents tetrazine amino acid used to evolve orthogonal aaRS/tRNA_{CUA} pair.

FIGS. 21A-21D present the results of SDS-page gel mobility shift assay and MS analysis confirming Tet-v4.0 derivatives incorporation into GFP and the labeling efficiency with sTCO reagent. FIG. 21A shows fidelity of Tet-v4.0 incorporation examined by reacting with sTCO-PEG5k and the mobility shift of the reacted proteins in SDS-page gel. ESI mass spectrometry analysis of purified GFP-Tet4.0 derivatives (black) and the reaction with 5-fold molar excess of sTCO for 10 minutes (gray) shows as expected 124 Da increase in mass corresponding to the addition of sTCO and loss of molecular nitrogen. No unreacted GFP-Tet-v4.0s were detected, verifying the reaction of genetically encoded Tet-v4.0 derivatives with sTCO was quantitative. Cal. Mass of GFP-wt: 27827.1 Da avg. FIG. 21B: GFP-Tet-v4.0_3-Me-Ph observed: 27955.1 Da avg, (expected: 27954.1 Da avg); GFP-Tet-v4.0_3-Me-Ph+sTCO observed: 28079.3 Da avg, (expected: 28078.2 Da avg). FIG. 21C: Tet-v4.0_3-F-Ph observed 27959.2 Da avg (expected: 27958.05 Da avg.) Tet-v4.0_3-F-Ph+sTCO observed: 28082.8 Da avg. (expected: 28082.1 Da avg.). FIG. 21D: Tet-4.0_3-CF₃-Ph observed 28008.2 Da avg (expected: 28008.1 Da avg.) Tet-4.0_3-CF₃-Ph+sTCO observed: 28132.5 Da avg. (expected: 28132.1 Da avg.). The peak at 27841.3 Da avg. observed due to near-cognate suppression of amber codon. The lower mass peak labeled with asterisk (*) is a loss of n-terminal methionine (-Met) and upper mass peaks are salt sodium and potassium adducts.

FIGS. 22A-22H verify stability and labeling efficiency of highly reactive Tet-v4.0 on protein in PBS (pH 7.1) at 4° C. and room temperature (RT) for 8 days. SDS-PAGE mobility shift assay by reacting with sTCO-PEG5k verified stability and reaction ability of (FIG. 22A) encoded Tet-v4.0Ph and (FIG. 22B) Tet-v4.0Pyr which were stored at 4° C. and RT for eight days in presence of 100 mM imidazole. ESI-Q-TOF mass spectrometry of purified GFP-Tet4.0-Ph/Pyr (black) and upon reaction with 5-fold molar excess of sTCO-OH for 10 minutes (gray)—confirmed that (FIGS. 22C and 22D) the Tet-v4.0Ph is highly stable inside protein at both 4° C. (FIG. 22E) and RT (FIG. 22F) after 8 days and reacts quantitively with sTCO; GFP-Tet-v4.0Ph observed: 27941.23 Da avg, (expected: 27940.1 Da avg); GFP-Tet-v4.0Ph+sTCO observed: 28065.2 Da avg, (expected: 28064.12 Da avg). FIG. 22G at 4° C., GFP-Tet-v4.0Pyr observed 27941.33 Da avg (expected: 27941.1 Da avg.) GFP-Tet-v4.0Pyr+sTCO-OH observed: 28066.24 Da avg. (expected: 28065.11 Da avg.). FIG. 22H shows Tet-v4.0Pyr degraded with time at room temperature. There was a small peak for reaction with sTCO and the majority of GFP-Tet-v4.0Pyr remains unreactive. GFP150-Tet-v4.0Pyr observed 27930.5 Da avg (expected: 27941.1 Da avg.) GFP-Tet-v4.0Pyr+sTCO observed: 27929.9 (unreactive) and 28066.76 Da avg. (expected: 28065.11 Da avg.) The unreacted single major peak at 27930.5 Da avg. indicates that the GFP-Tet-v4.0Pyr degraded under the following conditions and converted to its oxadiazole derivative which is 12 Da lower molecular mass than Tet-v4.0Pyr.

FIG. 23 compares eukaryotic protein expression and labeling of representative Tet-v4.0 ncAA derivatives.

FIGS. 24A-24C present the syntheses of representative fluorinated Tet-v4.0 ncAA: 2-amino-3-(6-(3,5-difluorophenyl)-1,2,4,5-tetrazin-3-yl)propanoic acid (Tet4.0-3,5-diF phenyl) (FIG. 24A); 2-amino-3-(6-(3,4,5-trifluorophenyl)-1,2,4,5-zin-3-yl)propanoic acid (Tet4.0-3,4,5-TriF phenyl) (FIG. 24B); and 2-amino-3-(6-(4-(trifluoromethyl)phenyl)-1,2,4,5-zin-3-yl)propanoic acid (Tet4.0 p-TriFmethylphenyl) (FIG. 24C).

FIG. 25 illustrates the spin-labeling methods described herein using representative Tet-v4.0 ncAAs (Tet-v4.0 methyl, phenyl, and 2-pyridyl).

FIG. 26 illustrates the synthesis of sTCO-PEG5k and sTCO-tM6, sTCO-tM5, and sTCO-tE5 spin labels.

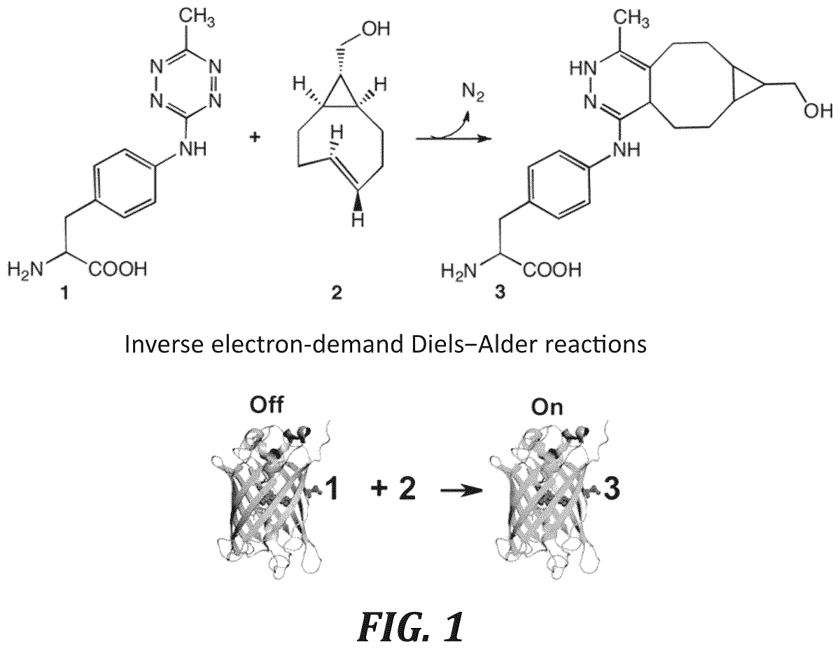

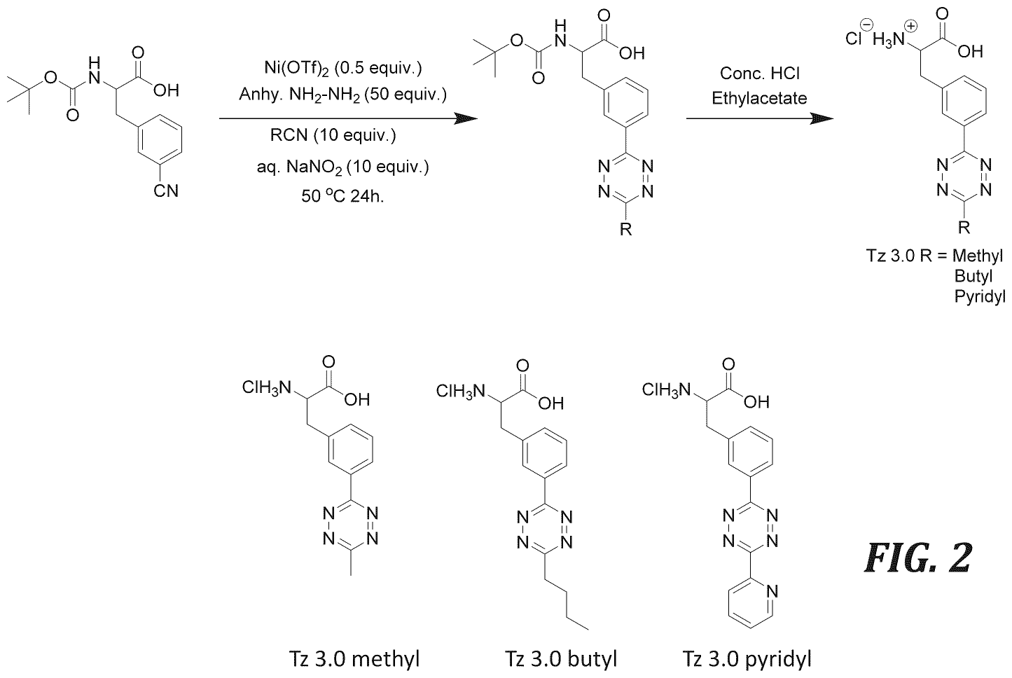

### DETAILED DESCRIPTION

The present disclosure provides compositions and methods for producing translational components that genetically encoded tetrazine amino acids in cells that meet the attributes needed for ideal bioorthogonal ligations. The components include orthogonal tRNAs, orthogonal aminoacyl-tRNA synthetases, orthogonal pairs of tRNAs/synthetases and tetrazine amino acids. Proteins containing tetrazine amino acids and methods of producing proteins with tetrazine amino acids in cells are also provided. In order to generate an ideal bioorthogonal reaction with the properties described the tetrazine amino acid reactivity is key to control. The tetrazine amino acid needs to be non-reactive to all biological conditions, in live cells, and in media, but highly reactive with its TCO partner. Orthogonal tRNAs/synthetases then need to be engineered to accept the tetrazine amino acid that contains these reactive properties. This results in tetrazine amino acid tRNA/synthetase pairs that function in cells to produce proteins with site-specifically incorporated tetrazine amino acids. The tetrazine amino acid containing proteins can then be used in cellulo, in vivo, or in vitro for ideal bioorthogonal ligations. The present disclosure provides composition and methods for producing tetrazine amino acid tRNA/synthetase pairs that are orthogonal in prokaryotic cells and eukaryotic cells. In certain embodiments, the methods for producing proteins with site-specifically incorporated tetrazine amino acids described herein are in cellulo methods. In other embodiments, the methods for producing proteins with site-specifically incorporated tetrazine amino acids described herein are cell-free methods.

The disclosure provides cells with translation components (e.g., pairs of orthogonal aminoacyl-tRNA synthetases (O-RSs) and orthogonal tRNAs (O-tRNAs) and individual components thereof, that are used in protein biosynthetic machinery to incorporate a tetrazine amino acid in a growing polypeptide chain, in a cell.

Compositions of the disclosure include a cell comprising an orthogonal aminoacyl-tRNA synthetase (O-RS), where the O-RS preferentially aminoacylates an orthogonal tRNA (O-tRNA) with at least one tetrazine amino acid (i.e., a tetrazine non-canonical amino acid as described herein) in the cell.

The cell also optionally includes the tetrazine amino acid(s). The cell optionally includes an orthogonal tRNA (O-tRNA), where the O-tRNA recognizes a selector codon and is preferentially aminoacylated with the tetrazine amino acid by the O-RS. In one aspect, the O-tRNA mediates the incorporation of the tetrazine amino acid into a protein with, for example, at least 45%, at least 50%, at least 60%, at least 75%, at least 80%, at least 90%, at least 95%, or 99% efficiency.

In another embodiment, the cell comprises a nucleic acid that comprises a polynucleotide that encodes a polypeptide of interest, where the polynucleotide comprises a selector codon that is recognized by the O-tRNA. In one aspect, the yield of the polypeptide of interest comprising the tetrazine amino acid is, e.g., at least 2.5%, at least 5%, at least 10%, at least 25%, at least 30%, at least 40%, 50% or more, of that obtained for the naturally occurring polypeptide of interest from a cell in which the polynucleotide lacks the selector codon. In another aspect, the cell produces the polypeptide of interest in the absence of the tetrazine amino acid, with a yield that is, e.g., less than 35%, less than 30%, less than 20%, less than 15%, less than 10%, less than 5%, less than 2.5%, of the yield of the polypeptide in the presence of the tetrazine amino acid.

The disclosure also provides a cell comprising an orthogonal aminoacyl-tRNA synthetase (O-RS), an orthogonal tRNA (O-tRNA), the tetrazine amino acid, and a nucleic acid that comprises a polynucleotide that encodes a polypeptide of interest. The polynucleotide comprises a selector codon that is recognized by the O-tRNA. In addition, the O-RS preferentially aminoacylates the orthogonal tRNA (O-tRNA) with the tetrazine amino acid in the cell, and the cell produces the polypeptide of interest in the absence of the tetrazine amino acid, with a yield that is, e.g., less than 30%, less than 20%, less than 15%, less than 10%, less than 5%, less than 2.5% of the yield of the polypeptide in the presence of the tetrazine amino acid.

Compositions that include a cell comprising an orthogonal tRNA (O-tRNA) are also a feature of the disclosure. Typically, the O-tRNA mediates incorporation of the tetrazine amino acid into a protein that is encoded by a polynucleotide that comprises a selection codon that is recognized by the O-tRNA in vivo. In one embodiment, the O-tRNA mediates the incorporation of the tetrazine amino acid into the protein with at least 45%, at least 50%, at least 60%, at least 75%, at least 80%, at least 90%, at least 95%, or even 99% efficiency.

In one aspect, the disclosure comprises a composition comprising a protein, wherein the protein comprises at least one tetrazine amino acid and at least one post-translational modification, wherein the at least one post-translational modification comprises attachment of a molecule comprising a second reactive group by a [4+2] cycloaddition to the at least one tetrazine amino acid comprising a first reactive group.

Thus, proteins (or polypeptides of interest) with at least one tetrazine amino acid are also a feature of the disclosure. In certain embodiments of the disclosure, a protein with at least one tetrazine amino acid includes at least one post-translational modification. In one embodiment, the at least one post-translational modification comprises attachment of a molecule (e.g., a dye, a polymer [e.g., a derivative of polyethylene glycol], a photocrosslinker, a cytotoxic compound, an affinity label, a derivative of biotin, a resin, a second protein or polypeptide, a metal chelator, a cofactor, a fatty acid, a carbohydrate, a polynucleotide (e.g., DNA, RNA) comprising a second reactive group by a [4+2] cycloaddition to the at least one tetrazine amino acid comprising a first reactive group. For example, the first reactive group is tetrazine moiety (e.g., a tetrazine moiety of a tetrazine non-canonical amino acid as described herein) and the second reactive group is an alkenyl moiety (e.g., a trans-cycloalkenyl moiety, such as a trans-cyclooctene (sTCO)). In certain embodiments, a protein of the disclosure includes at least one tetrazine amino acid comprising at least one post-translational modification. In certain embodiments, the post-translational modification is made in vivo in a cell.

Examples of a protein (or polypeptide of interest) include, but are not limited to, a cytokine, a growth factor, a growth factor receptor, an interferon, an interleukin, an inflammatory molecule, an oncogene product, a peptide hormone, a signal transduction molecule, a steroid hormone receptor, erythropoietin (EPO), insulin, human growth hormone, an alpha-1 antitrypsin, an angiostatin, an antihemolytic factor, an antibody, an apolipoprotein, an apoprotein, an atrial natriuretic factor, an atrial natriuretic polypeptide, an atrial peptide, a C—X—C chemokine, T39765, NAP-2, ENA-78, a Gro-a, a Gro-b, a Gro-c, an IP-10, a GCP-2, an NAP-4, an SDF-1, a PF4, a MIG, a calcitonin, a c-kit ligand, a cytokine, a CC chemokine, a monocyte chemoattractant protein-1, a monocyte chemoattractant protein-2, a monocyte chemoattractant protein-3, a monocyte inflammatory protein-1 alpha, a monocyte inflammatory protein-1 beta, RANTES, 1309, R83915, R91733, HCC1, T58847, D31065, T64262, a CD40, a CD40 ligand, a C-kit Ligand, a collagen, a colony stimulating factor (CSF), a complement factor 5a, a complement inhibitor, a complement receptor 1, a cytokine, DHFR, an epithelial neutrophil activating peptide-78, a GRO alpha/MGSA, a GRO beta, a GRO gamma, a MIP-1 alpha, a MIP-1 delta, a MCP-1, an epidermal growth factor (EGF), an epithelial neutrophil activating peptide, an erythropoietin (EPO), an exfoliating toxin, a Factor IX, a Factor VII, a Factor VIII, a Factor X, a fibroblast growth factor (FGF), a fibrinogen, a fibronectin, a G-CSF, a GM-CSF, a glucocerebrosidase, a gonadotropin, a growth factor, a growth factor receptor, a hedgehog protein, a hemoglobin, a hepatocyte growth factor (HGF), a hirudin, a human serum albumin, an ICAM-1, an ICAM-1 receptor, an LFA-1, an LFA-1 receptor, an insulin, an insulin-like growth factor (IGF), an IGF-I, an IGF-II, an interferon, an IFN-alpha, an IFN-beta, an IFN-gamma, an interleukin, an IL-1, an IL-2, an IL-3, an IL-4, an IL-5, an IL-6, an IL-7, an IL-8, an IL-9, an IL-10, an IL-11, an IL-12, a keratinocyte growth factor (KGF), a lactoferrin, a leukemia inhibitory factor, a luciferase, a neurturin, a neutrophil inhibitory factor (NIF), an oncostatin M, an osteogenic protein, an oncogene product, a parathyroid hormone, a PD-ECSF, a PDGF, a peptide hormone, a human growth hormone, a pleiotropin, a protein A, a protein G, a pyrogenic exotoxins A, B, or C, a relaxin, a renin, an SCF, a soluble complement receptor I, a soluble I-CAM 1, a soluble interleukin receptors, a soluble TNF receptor, a somatomedin, a somatostatin, a somatotropin, a streptokinase, a superantigen, a staphylococcal enterotoxin, an SEA, an SEB, an SEC1, an SEC2, an SEC3, an SED, an SEE, a steroid hormone receptor, a superoxide dismutase (SOD), a toxic shock syndrome toxin, a thymosin alpha 1, a tissue plasminogen activator, a tumor growth factor (TGF), a TGF-alpha, a TGF-beta, a tumor necrosis factor, a tumor necrosis factor alpha, a tumor necrosis factor beta, a tumor necrosis factor receptor (TNFR), a VLA-4 protein, a VCAM-1 protein, a vascular endothelial growth factor (VEGEF), a urokinase, a Mos, a Ras, a Raf, a Met; a p53, a Tat, a Fos, a Myc, a Jun, a Myb, a Rel, an estrogen receptor, a progesterone receptor, a testosterone receptor, an aldosterone receptor, an LDL receptor, a SCF/c-Kit, a CD40L/CD40, a VLA-4/VCAM-1, an ICAM-1/LFA-1, a hyalurin/CD44, a corticosterone, aprotein present in GenBank or other available databases, and/or a portion thereof. In one embodiment, the polypeptide of interest includes a transcriptional modulator protein (e.g., a transcriptional activator protein (such as GAL4), or a transcriptional repressor protein) or a portion thereof.

The disclosure also provides methods for producing, in a cell, at least one protein comprising at least one tetrazine amino acid (as well as proteins produced by such methods). The methods include growing, in an appropriate medium, a cell that comprises a nucleic acid that comprises at least one selector codon and encodes the protein. The cell also comprises an orthogonal tRNA (O-tRNA) that functions in the cell and recognizes the selector codon and an orthogonal aminoacyl tRNA synthetase (O-RS) that preferentially aminoacylates the O-tRNA with the tetrazine amino acid, and the medium comprises a tetrazine amino acid. In one embodiment, the O-RS aminoacylates the O-tRNA with the tetrazine amino acid (e.g., at least 45%, at least 50%, at least 60%, at least 75%, at least 80%, at least 90%, at least 95%, or even 99%).

In one embodiment, the method further includes incorporating into the protein the tetrazine amino acid, where the tetrazine amino acid comprises a first reactive group; and contacting the protein with a molecule (e.g., a dye, a polymer, [derivative of polyethylene glycol], a photocrosslinker, a cytotoxic compound, an affinity label, a derivative of biotin, a resin, a second protein or polypeptide, a metal chelator, a cofactor, a fatty acid, a carbohydrate, a polynucleotide [e.g., DNA, RNA]) that comprises a second reactive group. The first reactive group reacts with the second reactive group to attach the molecule to the tetrazine amino acid through a [4+2] cycloaddition. In one embodiment, the first reactive group is tetrazine moiety (e.g., the tetrazine moiety of a tetrazine non-canonical amino acid as described herein) and the second reactive group is an alkenyl moiety (e.g., a trans-cycloalkenyl moiety, such as sTCO).

In certain embodiments, the encoded protein comprises a therapeutic protein, a diagnostic protein, an industrial enzyme, or portion thereof. In one embodiment, the protein that is produced by the method is further modified through the tetrazine amino acid. For example, the tetrazine amino acid is modified through a [4+2] cycloaddition. In another embodiment, the protein produced by the method is modified by at least one post-translational modification (e.g., N-glycosylation, O-glycosylation, acetylation, acylation, lipid-modification, palmitoylation, palmitate addition, phosphorylation, glycolipid-linkage modification) in vivo.

In certain embodiments, the compositions and the methods of the disclosure include cells. The translation components of the disclosure can be derived from a variety of organisms (e.g., non-eukaryotic organisms, such as a prokaryotic organism, or an archaebacterium, or a eukaryotic organism).

Kits are also a feature of the disclosure. For example, a kit for producing a protein that comprises at least one tetrazine amino acid in a cell is provided, where the kit includes a container containing a polynucleotide sequence encoding an O-tRNA or an O-tRNA, and a polynucleotide sequence encoding an O-RS or an O-RS. In one embodiment, the kit further includes at least one tetrazine amino acid (i.e., a tetrazine non-canonical amino acid as described herein). In another embodiment, the kit further comprises instructional materials for producing the tetrazine-containing protein.

As used herein, the term “orthogonal” refers to a molecule (e.g., an orthogonal tRNA (O-tRNA) and/or an orthogonal aminoacyl tRNA synthetase (O-RS)) that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficient, less than 10% efficient, less than 5% efficient, or less than 1% efficient, of an orthogonal tRNA to function with an endogenous tRNA synthetase compared to an endogenous tRNA to function with the endogenous tRNA synthetase, or of an orthogonal aminoacyl-tRNA synthetase to function with an endogenous tRNA compared to an endogenous tRNA synthetase to function with the endogenous tRNA. The orthogonal molecule lacks a functional endogenous complementary molecule in the cell. For example, an orthogonal tRNA in a cell is aminoacylated by any endogenous RS of the cell with reduced or even zero efficiency, when compared to aminoacylation of an endogenous tRNA by the endogenous RS. In another example, an orthogonal RS aminoacylates any endogenous tRNA in a cell of interest with reduced or even zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., 50% efficiency, 60% efficiency, 70% efficiency, 75% efficiency, 80% efficiency, 90% efficiency, 95% efficiency, or 99% or more efficiency) to that of a corresponding tRNA/RS endogenous pair.

One advantage of the tetrazine non-canonical amino acids described herein is that they present additional chemical moieties that can be used to add additional molecules. These modifications can be made in vivo in a eukaryotic cell, or in vitro. Thus, in certain embodiments, the post-translational modification is through the tetrazine non-canonical amino acid. For example, the post-translational modification can be through a [4+2] cycloaddition reaction. Most reactions currently used for the selective modification of proteins involve covalent bond formation between nucleophilic and electrophilic reaction partners (e.g., the reaction of alpha-haloketones with histidine or cysteine side chains). Selectivity in these cases is determined by the number and accessibility of the nucleophilic residues in the protein. In proteins of the disclosure, other more selective reactions can be used in vitro and in vivo. This allows the selective labeling of virtually any protein with a host of reagents including fluorophores, crosslinking agents, polymers, saccharide derivatives and cytotoxic molecules.

Thus, this disclosure provides another highly efficient method for the selective modification of proteins, which involves the genetic incorporation of tetrazine amino acids into proteins in response to a selector codon. These tetrazine amino acid side chains can then be modified by a [4+2] cycloaddition reaction with strained alkenyl derivatives. Because this method involves a cycloaddition rather than a nucleophilic substitution, proteins can be modified with extremely high selectivity. This reaction can be carried out at room temperature in dilute aqueous conditions with excellent regio selectivity.

In one aspect, the disclosure provides tetrazine non-canonical amino acids (Tet ncAAs) useful for genetically encoding the tetrazine non-canonical amino acid in a polypeptide of interest. The tetrazine non-canonical amino acids of the disclosure are referred to herein as Tet-v4.0 amino acids. As used herein the term “tetrazine non-canonical amino acid (Tet ncAA) refers to a 1,2,4,5-tetrazine having an amino acid moiety at C3 (e.g., —CH₂CH(NHR^{N})(CO₂R^{C})) and optionally a substituent (other than hydrogen) at C6 (see, for example, formula (I)).

In one embodiment, the disclosure provides a tetrazine amino acid having formula (I):

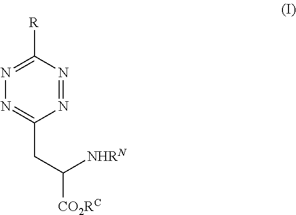

or a stereoisomer or salt thereof, wherein
R is selected from the group consisting of:
(a) a phenyl group substituted with a group selected from o-C1-C4 alkyl, m-C1-C4 alkyl, o-C1-C3 alkoxy, m-C1-C3 alkoxy, o-cyano, m-cyano, o-nitro, m-nitro, o- or m-primary amino (—NH₂), secondary amino (—NHR^{x}), or tertiary amino (—NHR^{x}R^{y}) (wherein R^{x} and R^{y} are independently C1-C6 alkyl), o-fluoro, m-fluoro, 3,5-difluoro, 3,4,5-trifluoro, o-trifluoromethyl, m-trifluoromethyl, and o-, m-, or p-C(═O)R^{z} (wherein R^{z} a counterion, hydrogen, or C1-C6 alkyl),
(b) a substituted or an unsubstituted heteroaryl group,
(c) a substituted or an unsubstituted heterocyclyl group,
(d) an amino C1-C6 alkyl group,
(e) a thio C1-C6 alkyl group,
(f) a carboxylate group,
(g) a sulfonate group, and
(h) an amide group;
R^{C} is hydrogen, a counter ion, or a carboxyl protecting group; and
R^{N} is hydrogen or an amine protecting group.

In certain embodiments, R is a substituted phenyl group. Suitable substituents include C1-C6 alkyl (e.g., methyl), C1-C3 haloalkyl (trifluoromethyl), halo (e.g., fluoro, chloro), hydroxy, C1-C3 alkoxy (e.g., methoxy), cyano, nitro, —CO₂R^{z}, where z is a counterion, hydrogen, or C1-C6 alkyl group, and primary (—NH₂), secondary (—NHR^{x}), and tertiary amine (—NR^{x}R^{y}) (where R^{x} and R^{y} are independently C1-C6 alkyl). The phenyl groups may include one or more substituents at the o-, m-, and/or p- (i.e., 2-, 3-, and/or 4-) positions. Representative substituted phenyl groups include phenyl groups substituted with m-methyl (3-methyl), o-methoxy (2-methoxy), m-methoxy (3-methoxy), o-cyano (2-cyano), m-cyano (3-cyano), o-nitro (2-nitro), m-nitro (3-nitro), o-fluoro (2-fluoro), m-fluoro (3-fluoro), 3,5-difluoro, 3,4,5-trifluoro, o-hydroxy (2-hydroxy), m-hydroxy (3-hydroxy), o-NH₂, (2-NH₂), m-NH₂ (3-NH₂), m-C(═O)CH₃ (2-acetyl), o-trifluoromethyl (2-trifluoromethyl), and m-trifluoromethyl (3-trifluoromethyl).

In further embodiments, R is an unsubstituted heteroaryl group or a substituted heteroaryl group. As used herein, the term “heteroaryl” refers to a monocyclic heteroaryl group that is a 3- to 6-membered carbocyclic group containing one or more nitrogen, oxygen, or sulfur atoms in the carbocyclic ring. Suitable heteroaryl groups include pyridyl (e.g., 2-, 3-, and 4-pyridyl), furanyl (2- and 3-furanyl), thiophenyl (2- and 3-thiophenyl), and oxazolyl (e.g., 2-, 3-, 4-, and 5-oxazolyl) groups. Suitable substituents include C1-C6 alkyl (e.g., methyl), C1-C3 haloalkyl (trifluoromethyl), halo (e.g., fluoro, chloro), hydroxy, C1-C3 alkoxy (e.g., methoxy), cyano, nitro, and primary (—NH₂), secondary (—NHR^{x}), and tertiary amine (—NR^{x}R^{y}) (where R^{x} and R^{y} are independently C1-C6 alkyl). The heteroaryl groups may include one or more substituents (e.g., at one or more of each ring position).

In other embodiments, R is a substituted or an unsubstituted heterocyclyl group. As used herein, the term “heterocyclyl” refers to a monocyclic heterocyclyl group that is a 3- to 6-membered carbocyclic group containing one or more nitrogen, oxygen, or sulfur atoms in the carbocyclic ring. Suitable heterocyclyl groups include pyrrolidinyl (e.g., 2- and 3-pyrrolidinyl), piperadinyl (e.g., 2-, 3-, and 4-piperadinyl), piperazinyl (e.g., 2- and 3-piperazinyl), tetrahydrofuranyl (e.g., 2- and 3-tetrahydrofuranyl), tetrahydropyranyl (e.g., 2-, 3-, and 4-tetrahydropyranyl), tetrahydrothiophenyl (e.g., 2- and 3-tetrahydrothiophenyl), and oxazolydinyl (e.g., 2-, 3-, 4-, and 5-oxazolydinyl) groups. Suitable heterocyclyl groups include dihydropyran (DHP) groups. Suitable substituents include C1-C6 alkyl (e.g., methyl), C1-C3 haloalkyl (trifluoromethyl), halo (e.g., fluoro, chloro), hydroxy, C1-C3 alkoxy (e.g., methoxy), cyano, nitro, and primary (—NH₂), secondary (—NHR^{x}), and tertiary amine (—NR^{x}R^{y}) (where R^{x} and R^{y} are independently C1-C6 alkyl).

In another embodiment, R is an amino C1-C6 alkyl group. Representative amino C1-C6 alkyl groups include aminomethyl, aminoethyl, aminopropyl, aminobutyl, aminopentyl, and aminohexyl groups. In one embodiment, the amino C1-C6 alkyl group is an aminobutyl (—NH(CH₂)₃CH₃) group.

In a further embodiment, R is a thio C1-C6 alkyl group. Representative thio C1-C6 alkyl groups include thiomethyl, thioethyl, thiopropyl, thiobutyl, thiopentyl and thiohexyl groups. In one embodiment, the thio C1-C6 alkyl group is a thiomethyl (—SCH₃) group.

In another embodiment, R is a carboxylate group (e.g., —CO₂R^{z}, where z is a counterion, hydrogen, or C1-C6 alkyl group). Representative carboxylate groups include methyl carboxylate (—CO₂CH₃) and ethyl carboxylate (—CO₂CH₂CH₃) groups. In one embodiment, the carboxylate group is an ethyl carboxylate group.

In another embodiment, R is a sulfonate group (e.g., —SO₃R^{z}, where z is a counterion, hydrogen, or C1-C6 alkyl group). Representative sulfonate groups include sulfonate (—SO₃⁻) and methyl sulfonate (—SO₂OCH₃) and ethyl carboxylate (—CO₂CH₂CH₃) groups.

In a further embodiment, R is an amide group. Representative amide groups include C1-C6 alkyl amide groups (e.g., methyl amide (—C(═O)NHCH₃), butyl amide (—C(═O)NH(CH₂)₃CH₃)).

Carboxyl protecting groups, amine protecting groups, and counter ions include those known in the art. Suitable carboxyl protecting groups and amine protecting groups are described in Protective Groups in Organic Synthesis, T. W. Greene, John Wiley & Sons, 1981, expressly incorporated herein by reference in its entirety.

Suitable carboxyl protecting groups include ester, amide, and hydrazine groups. Representative ester groups include substituted methyl esters (e.g., methoxymethyl, methylthiomethyl, tetrahydropyranyl, tetrahydrofuranyl, methoxyethoxymethyl, benzyloxymethyl, phenacyl, p-bromophenacyl, α-methylphenacyl, p-methoxyphenacyl, diacylmethyl, N-phthalimidomethyl, and ethyl), 2-substituted ethyl esters (e.g., 2,2,2-trichloroethyl, 2-haloethyl, ω-chloroalkyl, 2-(trimethylsilyl)ethyl, 2-methylthioethyl, 2-(p-nitrophenylsulfenyl)ethyl, 2-(p-toluenesulfonyl)ethyl, 1-methyl-1-phenylethyl, t-butyl, cyclopentyl, cyclohexyl, allyl, cinnamyl, phenyl, p-methylthiophenyl, and benzyl), substituted benzyl esters (e.g., triphenylmethyl, diphenylmethyl, bis(o-nitrophenyl)methyl, 9-anthrylmethyl, 2-(9,10-dioxo)anthrylmethyl, 5-dibenzosuberyl, 2,4,6-trimethylbenzyl, p-bromobenzyl, o-nitrobenzyl, p-nitrobenzyl, p-methoxybenzyl, piperonyl, and 4-picolyl), silyl esters (e.g., trimethylsilyl, triethylsilyl, t-butyldimethylsilyl, i-propyldimethylsilyl, and phenyldimethylsilyl), activated esters (e.g., S-t-butyl, S-phenyl, S-2-pyridyl, N-hydroxypiperidinyl, N-hydroxysuccinimidoyl, N-hydroxyphthalimidoyl, and N-hydroxybenzotriazolyl), stannyl esters (e.g., triethylstannyl, tri-n-butylstannyl), and other esters (e.g., O-acyl oximes, 2,4-dinitrophenylsulfenyl, 2-alkyl-1,3-oxazolidines, 4-alkyl-5-oxo-1,3-oxazolidines, and 5-alkyl-4-oxo-1,3-dioxolanes). Representative amides include N,N-dimethyl, pyrrolidinyl, piperidinyl, o-nitrophenyl, 7-nitroindolyl, and 8-nitrotetrahydroquinolyl. Representative hydrazines include N-phenylhydrazide and N,N′-diisopropylhydrazide.

Suitable amino protecting groups include carbamate and amide groups. Representative carbamate groups include alkyl and aryl carbamate groups (e.g., methyl and substituted methyl, substituted ethyl, substituted propyl and isopropyl, t-butyl, cyclobutyl, cyclopentyl, cyclohexyl, 1-adamantyl, vinyl, allyl, cinnamyl, phenyl, and benzyl). Representative amide groups include N-formyl, N-acetyl, substituted N-propionyl, cyclic imides, N-alkyl amide (e.g., N-allyl, N-phenacyl), amino acetals, N-benzyl amides, imine derivatives, enamine derivatives, N-heteroatom derivatives, N-metal derivatives (e.g., N-borane, N-copper, N-zinc), N—N derivatives (e.g., N-nitro, N-nitroso), N—P derivatives (e.g., phosphinyl, phosphoryl) N—Si derivatives (e.g., N-trimethylsilyl), and N—S derivatives (e.g., N-sulfenyl, N-sulfonyl).

In certain embodiments, the compounds of the disclosure are amino acids and maybe exist in neutral (e.g., —NH₂ and —CO₂H) or ionic form (e.g., —NH₃⁺ and —CO₂⁻) depending on the pH of the environment. It will be appreciated that the compounds of the disclosure include a chiral carbon center and that the compounds of the disclosure can take the form of a single stereoisomer (e.g., L or D isomer) or a mixture of stereoisomers (e.g., a racemic mixture or other mixture). It will be appreciated that the individual stereoisomers and mixtures of isomers are useful in methods of the disclosure for incorporating tetrazine-containing residues into proteins and polypeptides.

The tetrazine amino acids described herein are useful in the methods for genetically encoding a polypeptide of interest.

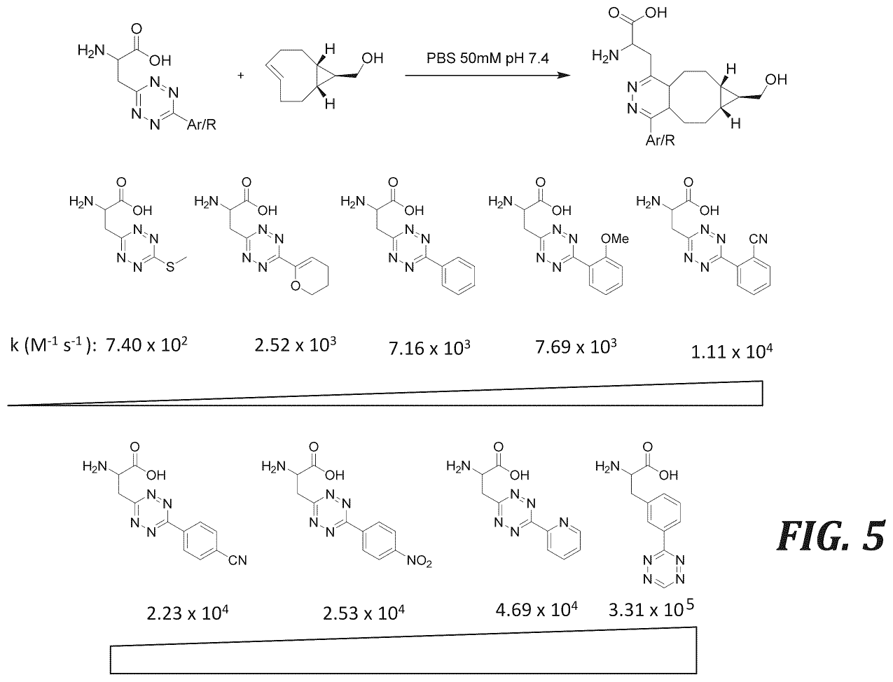

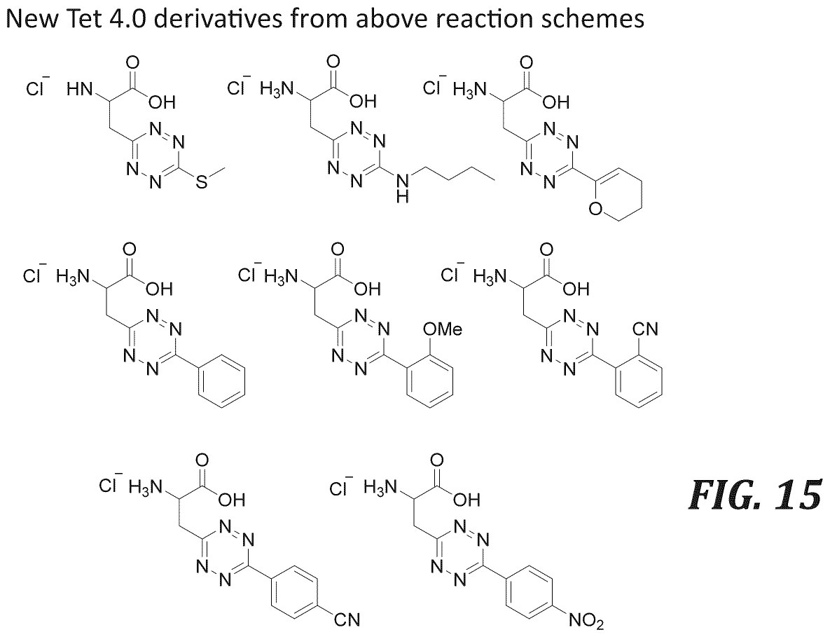

Representative tetrazine amino acids of the present disclosure include those shown in FIGS. 5, 15, and 16.

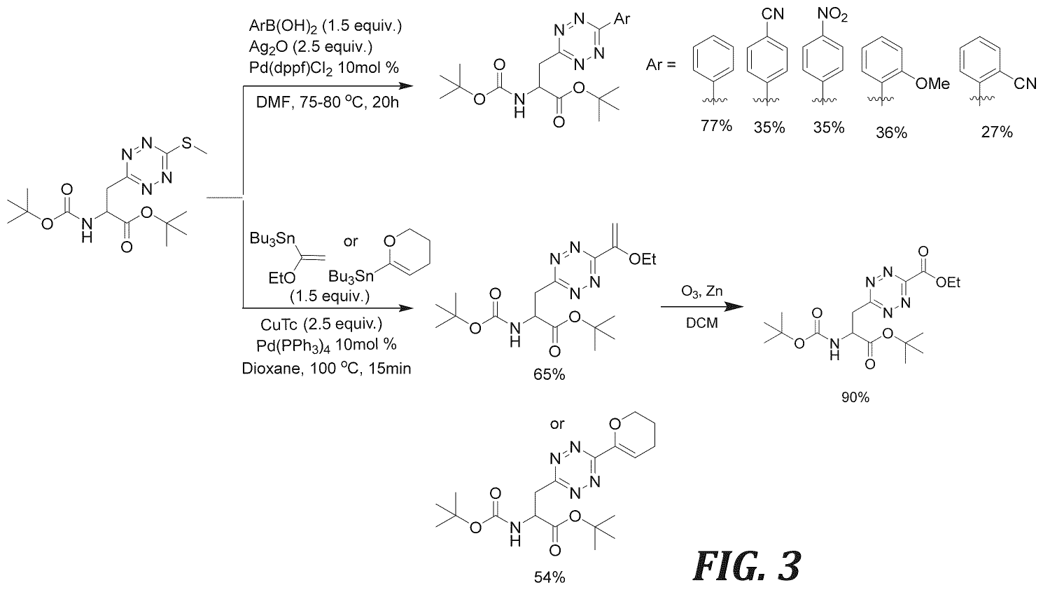

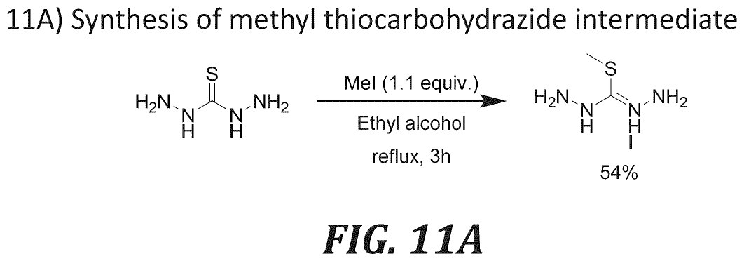

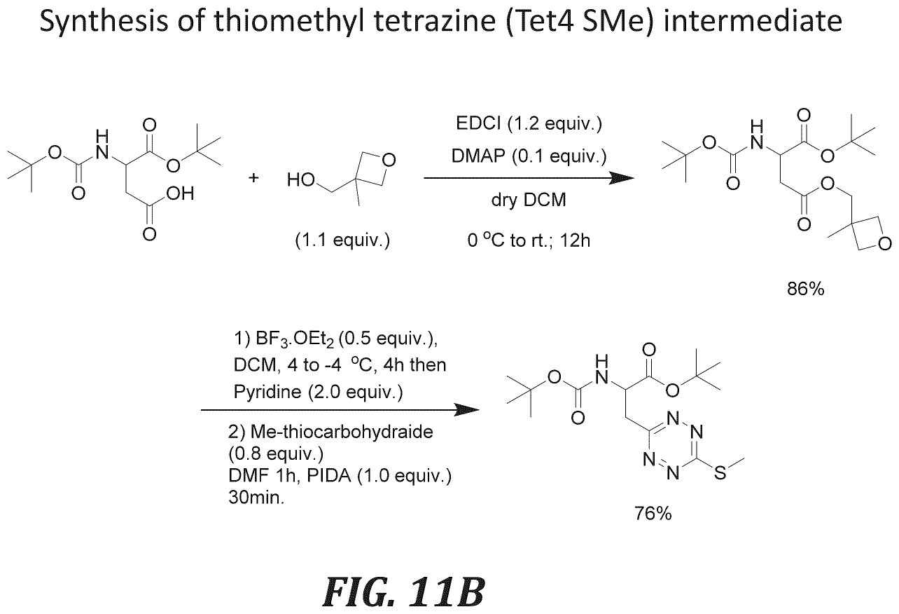

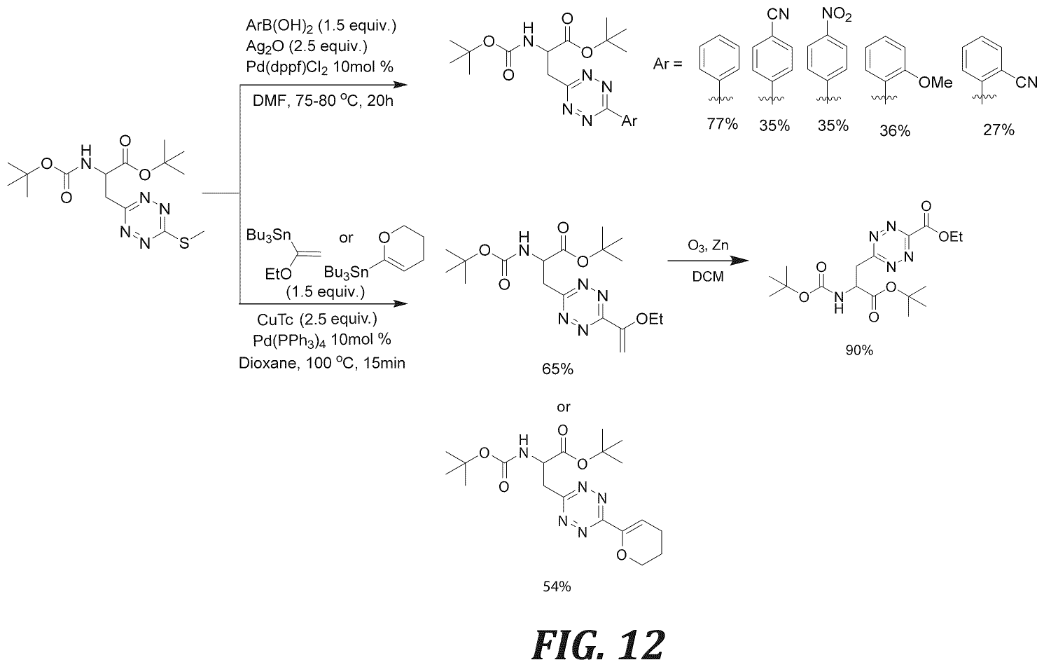

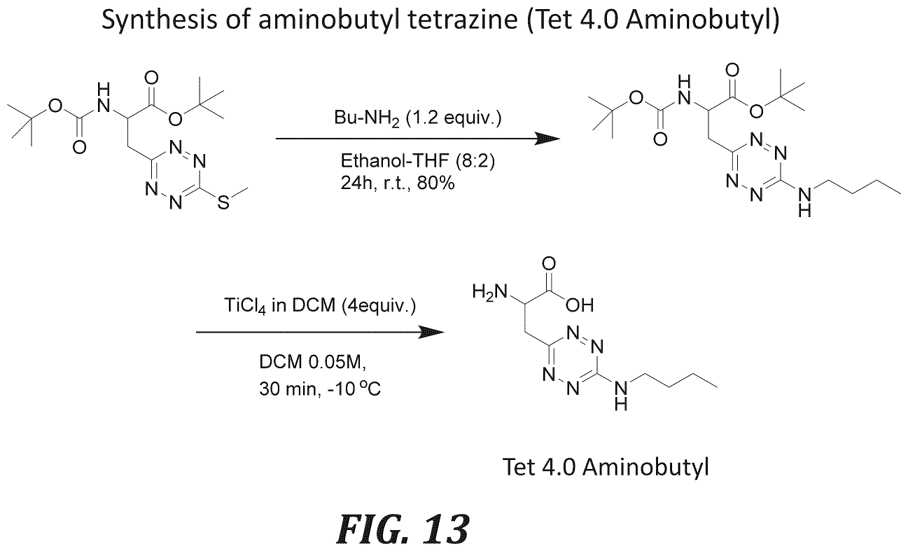

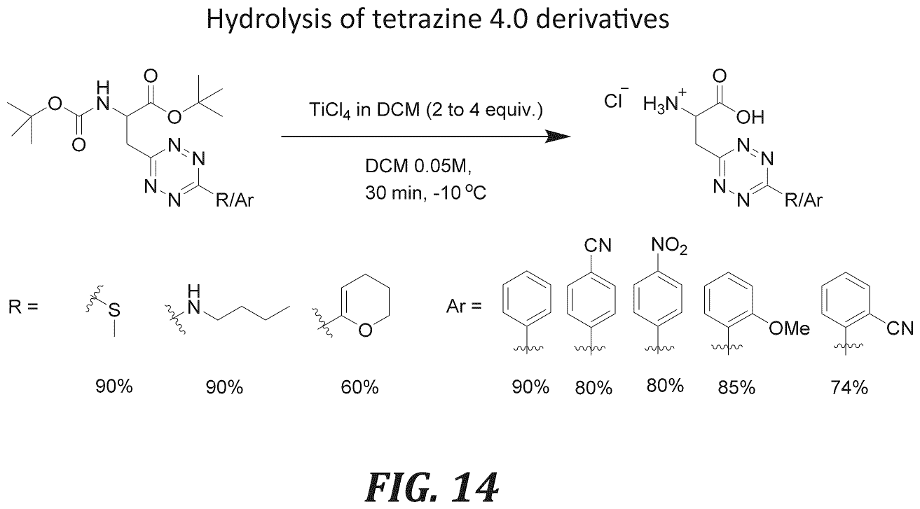

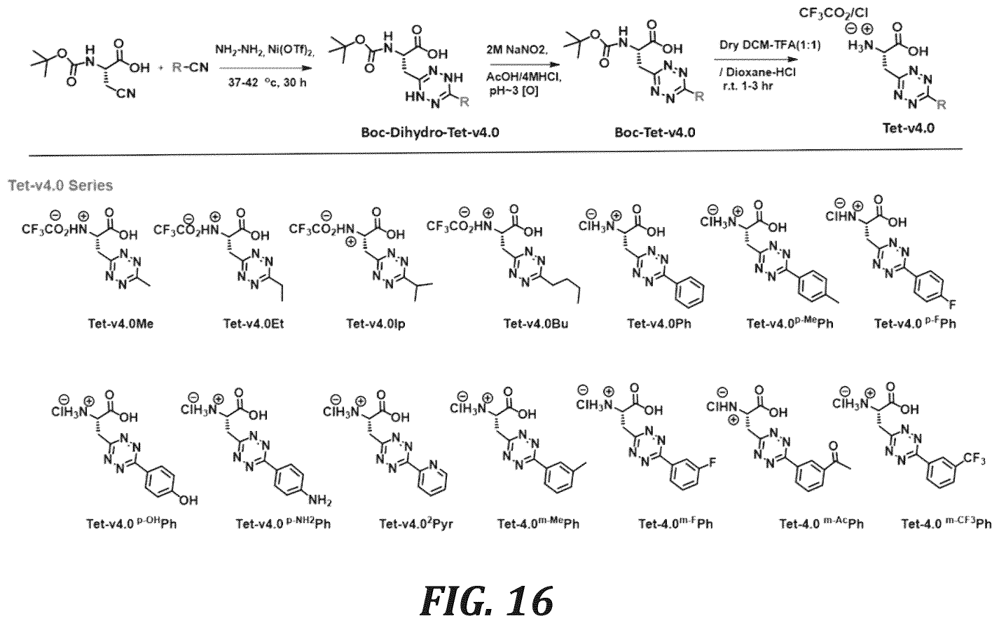

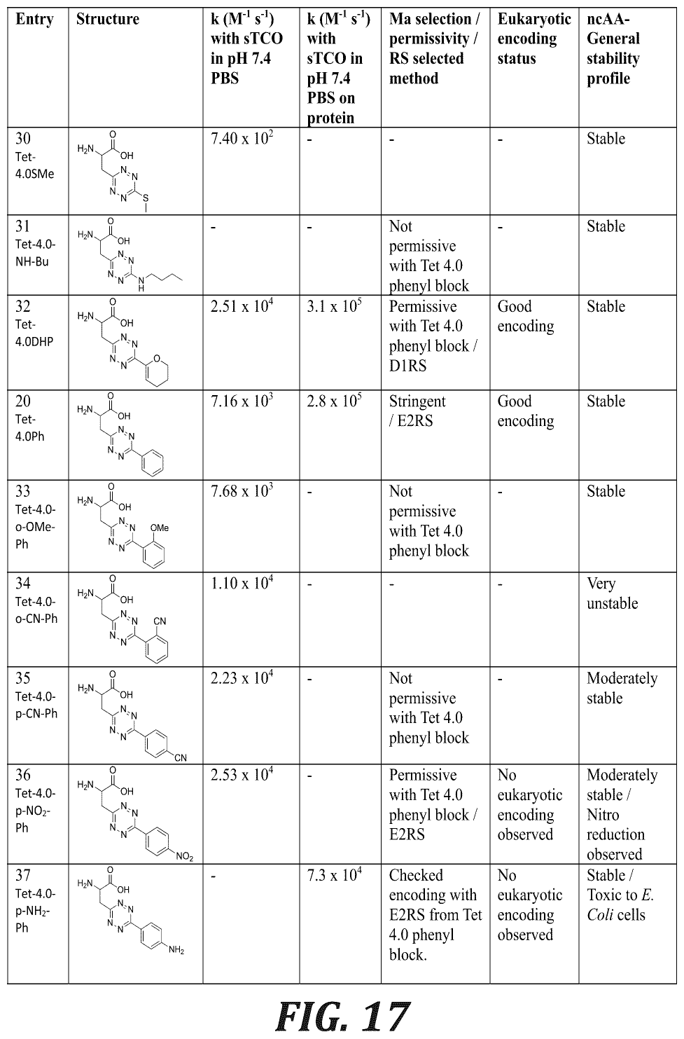

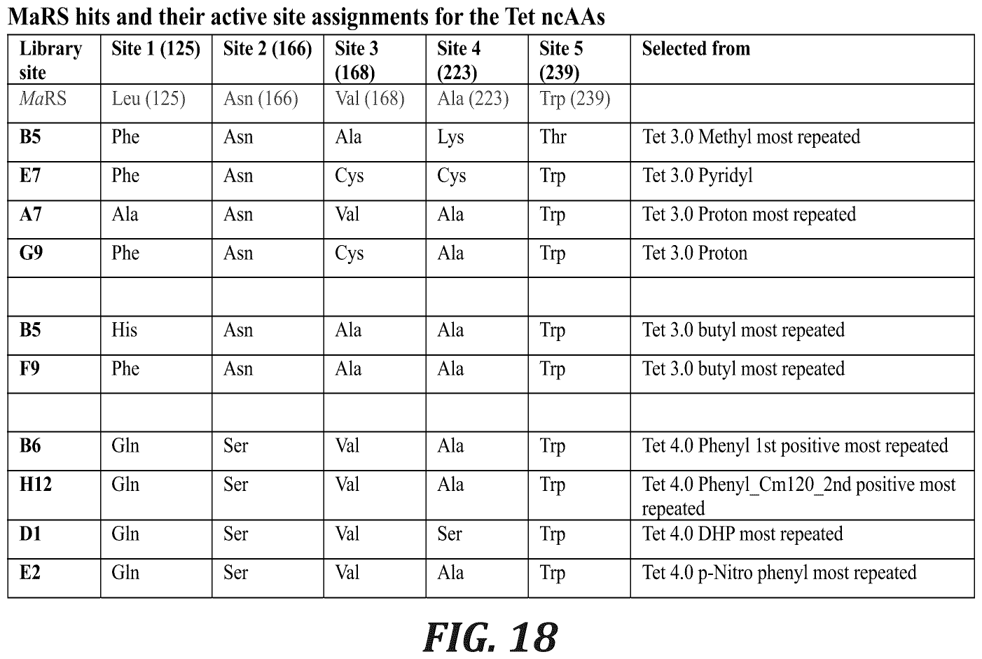

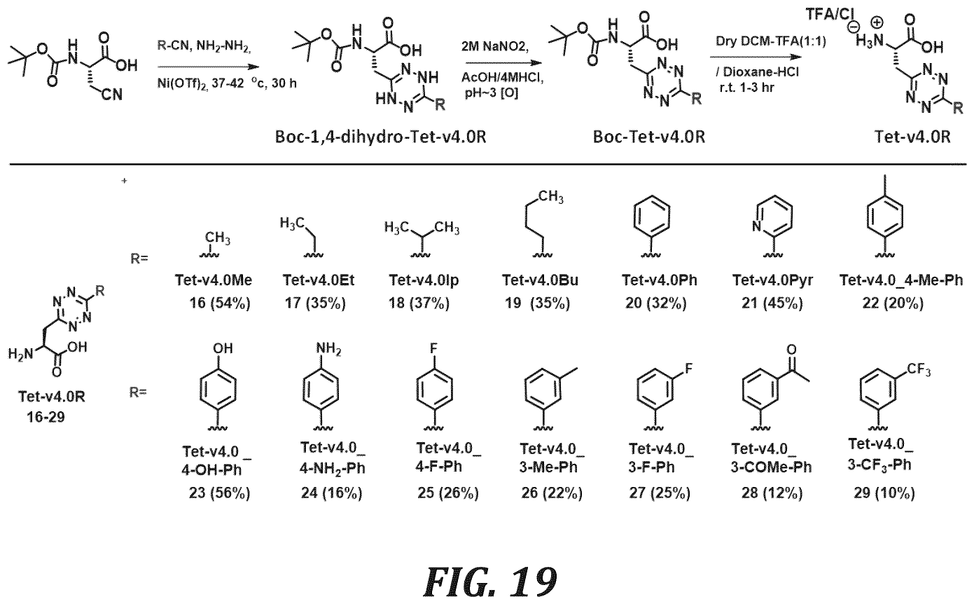

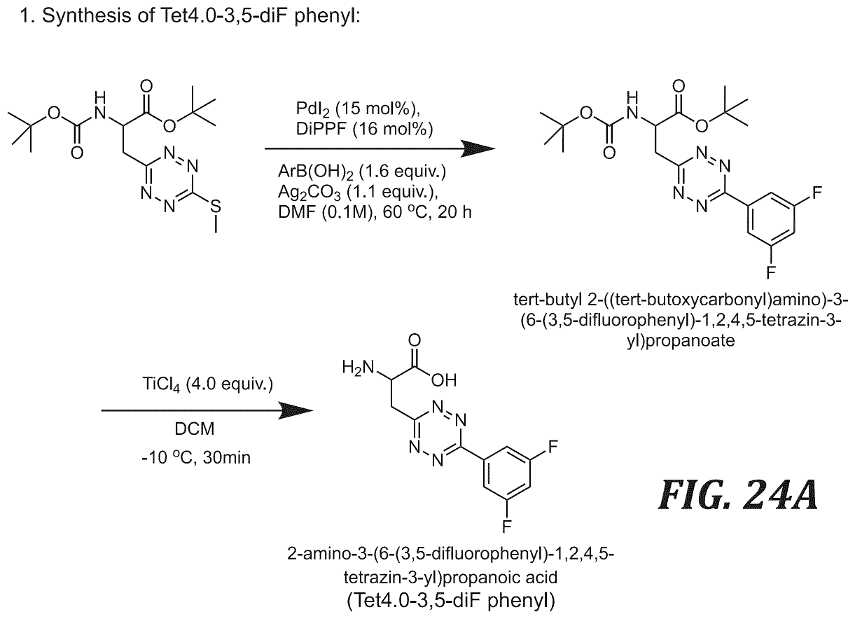

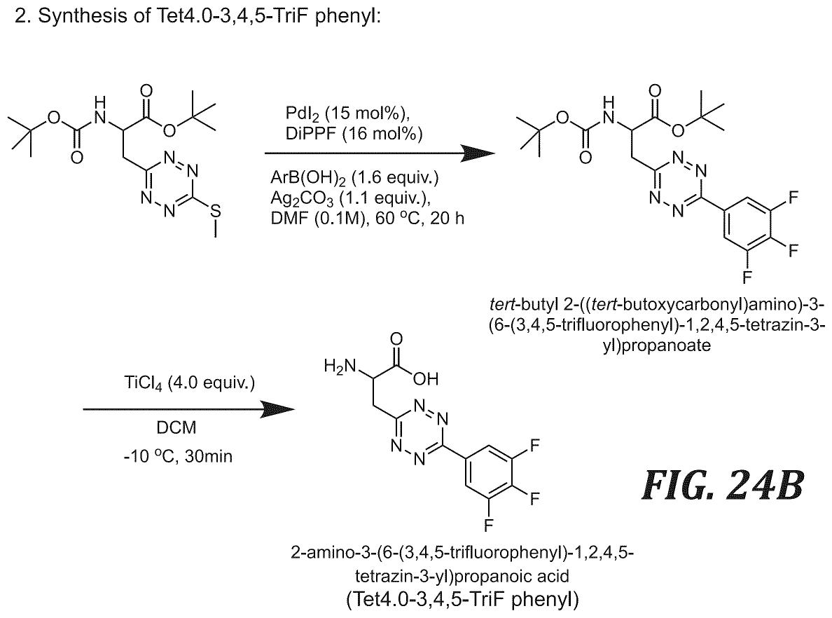

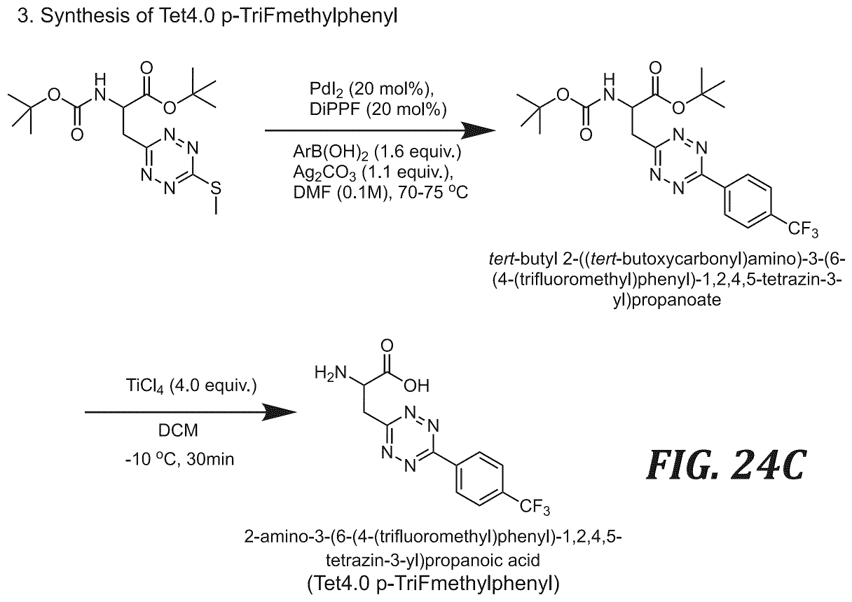

The preparation of a representative tetrazine amino acid of the present disclosure are illustrated in FIGS. 3, 12-14, 16, 19, and 24A-24C. FIGS. 11A and 11B are schematic illustrations of the preparation of intermediates useful in the preparation of representative tetrazine amino acids. FIG. 11A illustrates the synthesis of a methyl thiocarbohydrazide intermediate. FIG. 11B illustrates the synthesis of a thiomethyl tetrazine (Tet-v4.0 SMe) intermediate. FIG. 12 is a schematic illustration of the preparation of representative tetrazine (Tet-v4.0) amino acids from the intermediate shown in FIG. 11B. FIG. 13 is a schematic illustration of the preparation of a representative tetrazine amino acid (Tet-v4.0 aminobutyl) from the intermediate shown in FIG. 11B. FIG. 14 is a schematic illustration of the preparation of representative tetrazine amino acids (Tet-v4.0) via hydrolysis of protected carboxylic acid and amino groups of tetrazine amino acid precursors. FIGS. 16, 19, and 24A-24C present general synthetic schemes for the preparation of representative tetrazine amino acids (Tet-v4.0).

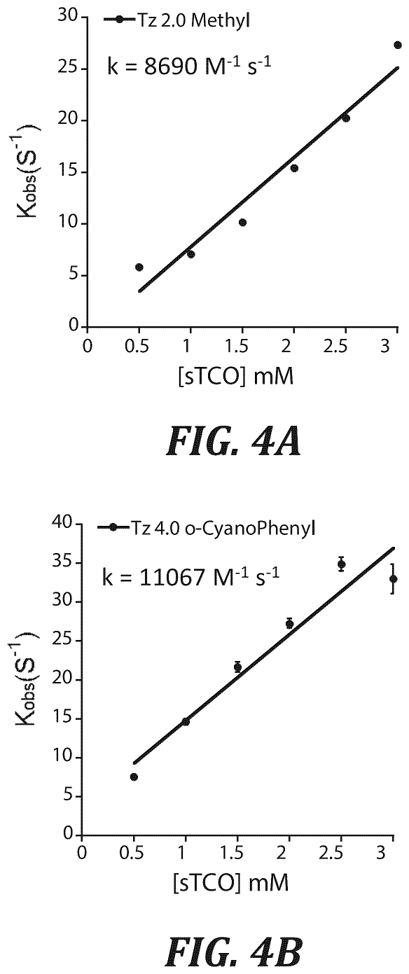

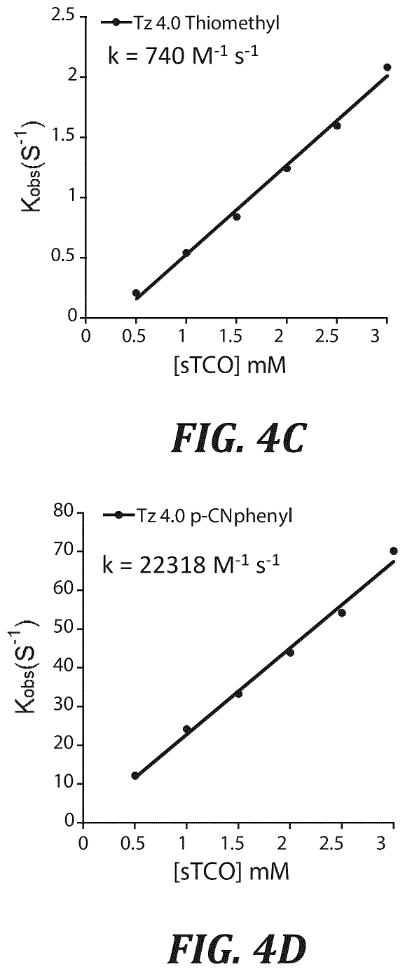

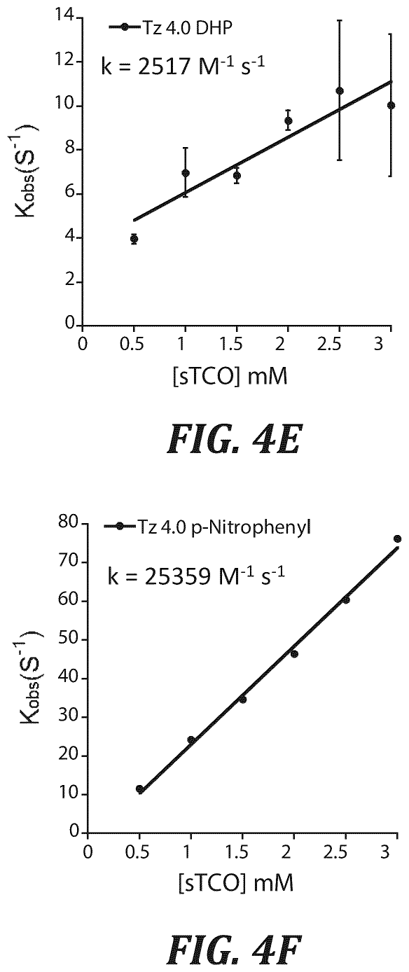

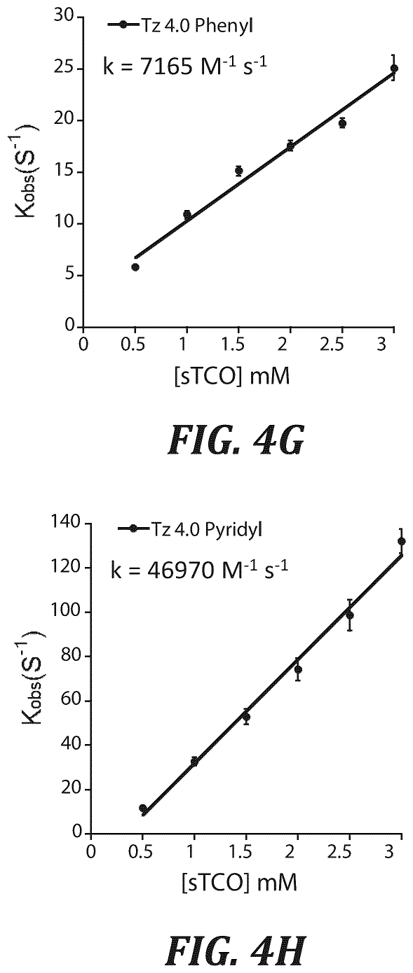

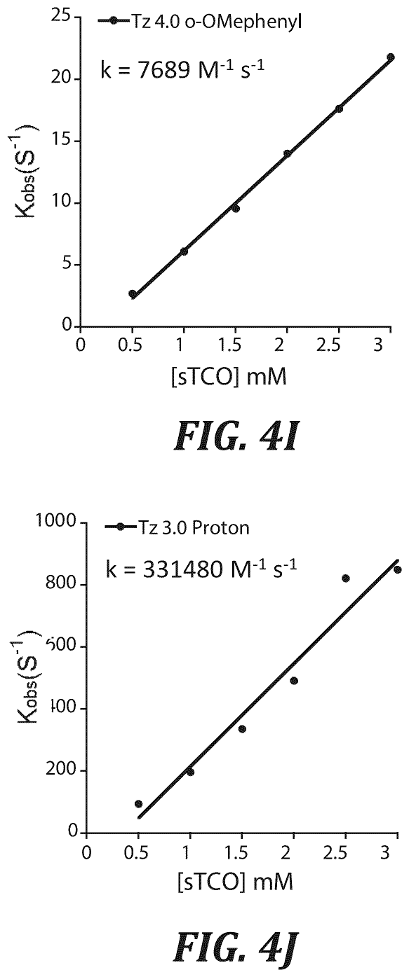

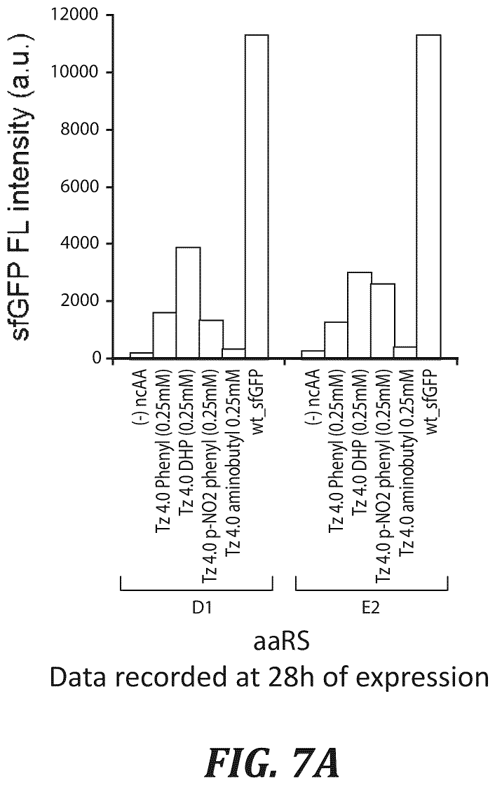

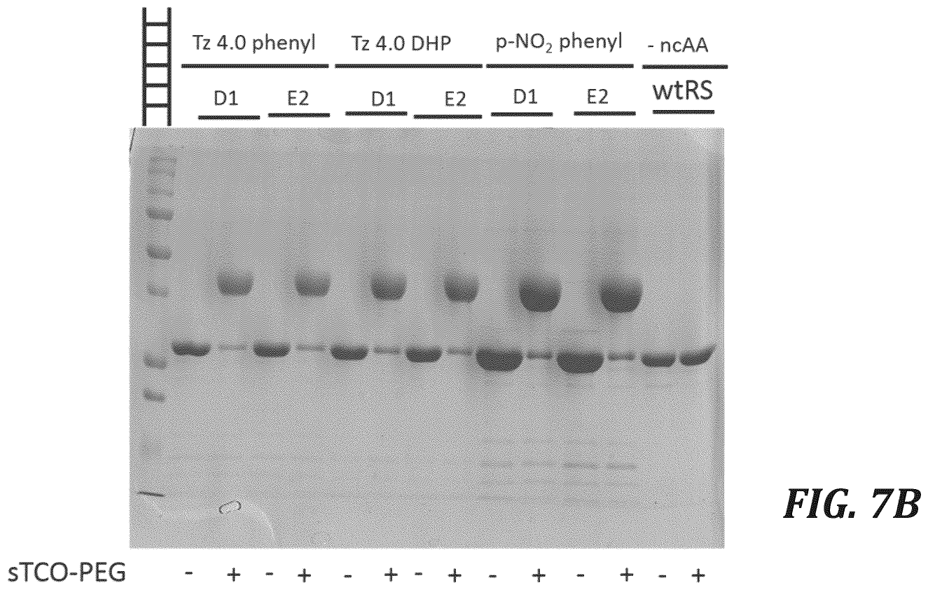

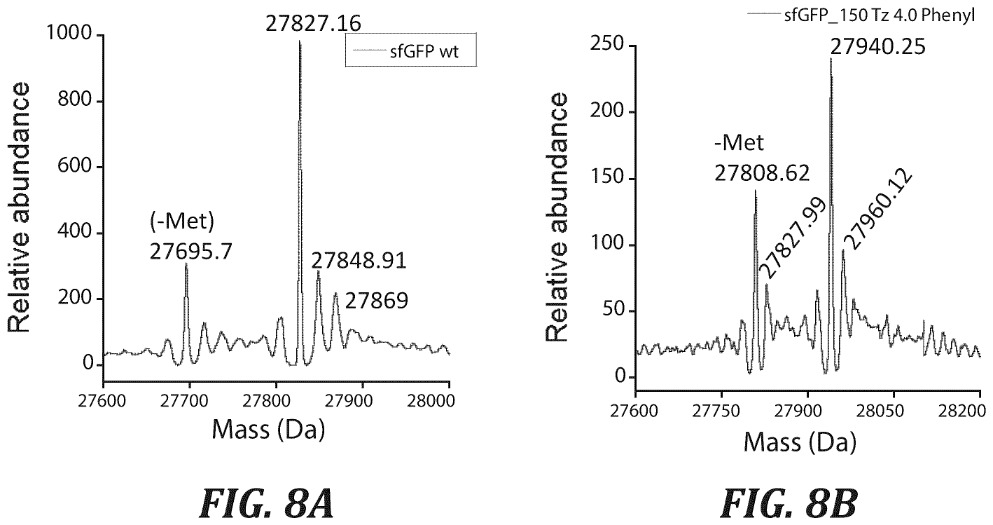

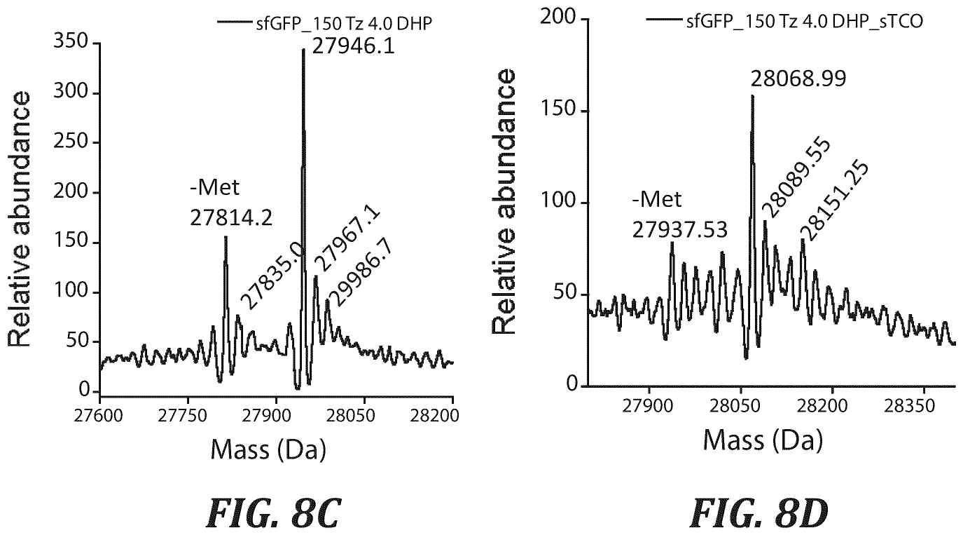

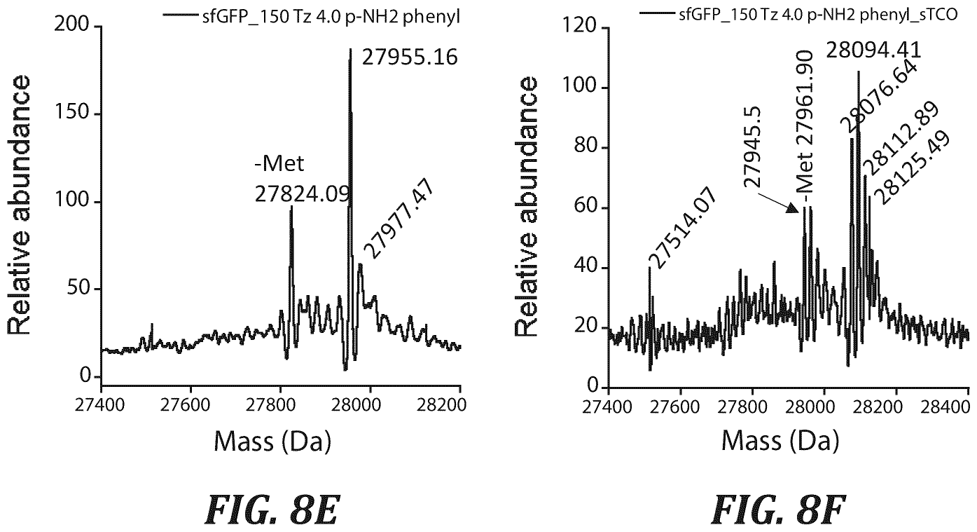

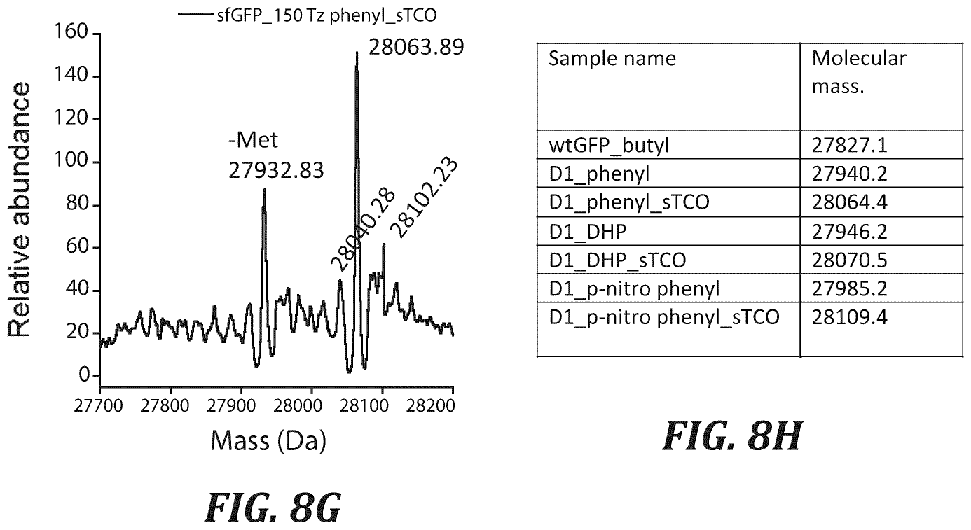

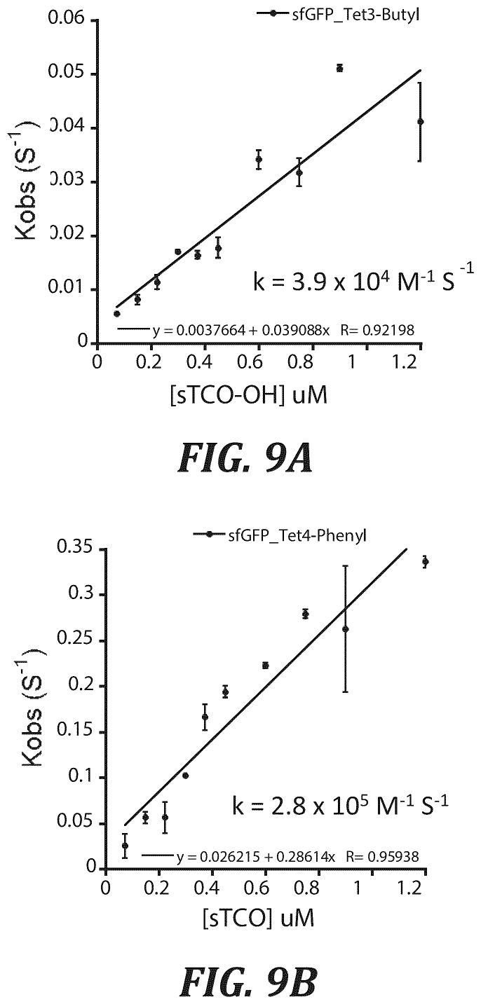

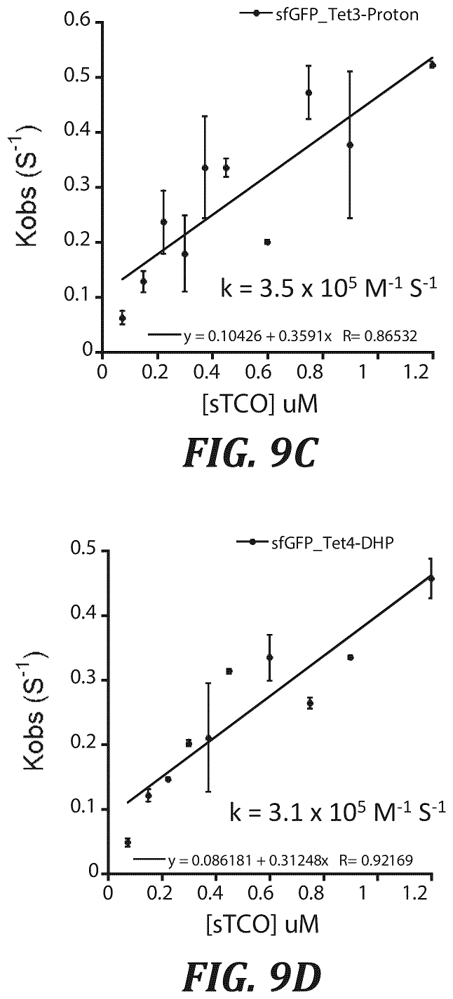

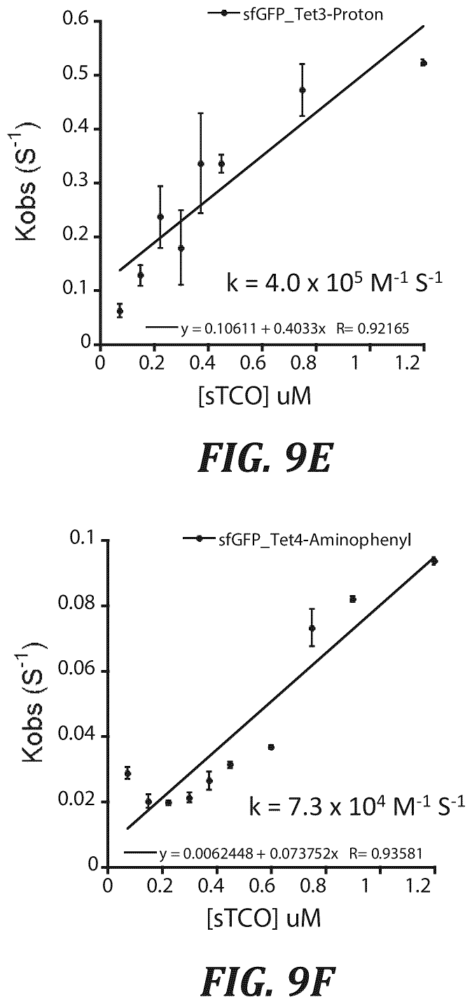

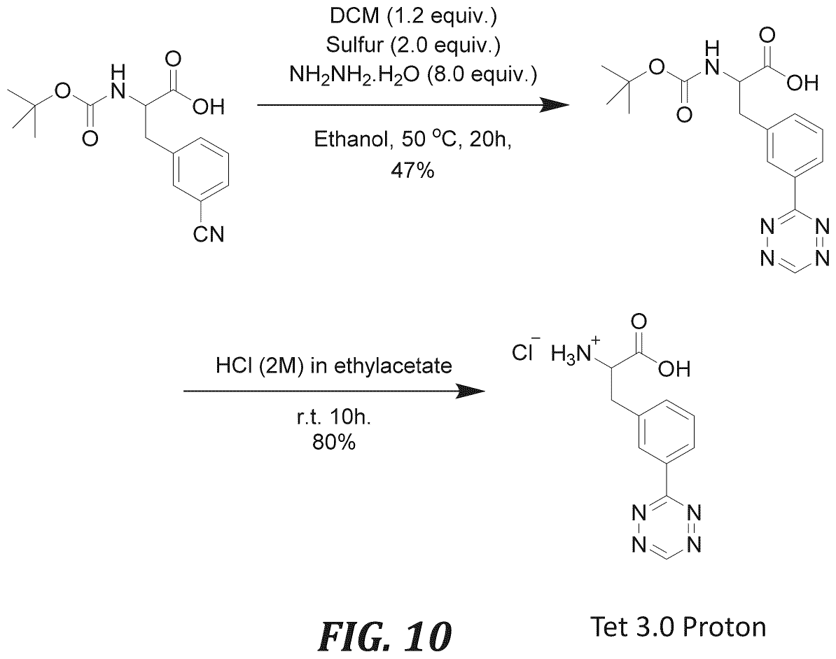

The properties of representative tetrazine amino acids of the present disclosure and their protein products are shown in FIGS. 4A-4J, 5, 7A and 7B, and 9A-9F. FIGS. 4A-4J compare stop-flow kinetics measurements of representative tetrazine (Tet-v4.0) non-canonical amino acids in sTCO reaction relative to tetrazine (Tet-v2.0 methyl and Tet-v3.0 hydrogen) non-canonical amino acids. FIG. 5 compares reaction kinetics of representative tetrazine (Tet-v4.0) non-canonical amino acids in sTCO reaction relative to a representative tetrazine (Tet-v3.0 hydrogen) non-canonical amino acid. FIGS. 7A and 7B show expression (7A) and characterization (7B) of sfGFP expressed with selected aaRS and representative tetrazine (Tet-v4.0) non-canonical amino acids. FIGS. 8A-8H compare mass spectrometric characterization of sfGFP with representative tetrazine (Tet-v4.0) non-canonical amino acids with and without reaction of sTCO. FIGS. 9A-9F compare fluorescence kinetics measurements of representative sfGFP-tetrazine (Tet-v4.0) non-canonical amino acids in sTCO reaction relative to sfGFP-tetrazine (Tet-v3.0 hydrogen and butyl) non-canonical amino acids.

In another aspect, the disclosure provides methods for making proteins or polypeptides that include tetrazine-containing residues derived from tetrazine amino acids of formula (I). In one embodiment, the method includes incorporating a tetrazine amino acid of the disclosure into the protein or polypeptide. Tetrazine amino acids can be incorporated into a protein or polypeptide by conventional synthetic techniques (e.g., peptide synthesis, such as solid phase peptide synthesis). Alternatively, the tetrazine amino acid can be incorporated into a protein or polypeptide by genetic encoding, as described herein in detail. In one embodiment, the disclosure provides a method for genetically encoding a protein or polypeptide of interest that includes incorporating a tetrazine amino acid of the disclosure into the protein or polypeptide by genetic encoding.

In a further aspect, the disclosure provides a protein or polypeptide that includes at least one tetrazine amino acid residue is provided. The tetrazine amino acid residue is derived from a tetrazine amino acid of the disclosure. In one embodiment, the disclosure provides a protein or polypeptide, comprising at least one tetrazine amino acid residue, wherein the tetrazine amino acid residue is incorporated into the protein or polypeptide by genetic encoding of the protein or polypeptide using a tetrazine amino acid of the disclosure.

In another aspect, the disclosure provides a post-translationally modified composition protein or polypeptide (e.g. composition) comprising a protein or polypeptide that comprises at least one tetrazine amino acid residue and at least one post-translational modification, wherein the at least one post-translational modification comprises attachment of a molecule comprising a second reactive group by a [4+2] cycloaddition to the at least one tetrazine amino acid residue comprising a first reactive group.

In certain embodiments, the disclosure provides a composition comprising a protein or polypeptide, wherein the protein or polypeptide comprises at least one tetrazine amino acid residue comprising a first reactive group and at least one post-translational modification, wherein the tetrazine amino acid residue is derived from a tetrazine-containing compound, wherein the at least one post-translational modification comprises attachment of a molecule comprising a second reactive group by a [4+2] cycloaddition reaction to the at least one tetrazine amino acid residue comprising the first reactive group.

In other embodiments, the disclosure provides a composition comprising a protein or polypeptide, wherein the protein or polypeptide comprises at least one tetrazine amino acid residue comprising a first reactive group and at least one post-translational modification, wherein the tetrazine amino acid residue is derived from genetic encoding of the protein or polypeptide with a tetrazine-containing compound, wherein the at least one post-translational modification comprises attachment of a molecule comprising a second reactive group by a [4+2] cycloaddition reaction to the at least one tetrazine amino acid residue comprising the first reactive group.

In certain of the embodiments, the first reactive group is the tetrazine group of the tetrazine amino acid residue and the second reactive group is a suitably reactive group reactive (alkyne or alkene, such as a strained alkyne or strained alkene, or a cyclic alkene or cyclic alkyne).

In another aspect of the disclosure, a kit for in cellulo production of a tetrazine-labeled protein or a tetrazine-labeled polypeptide is provided. In certain embodiments, the kit includes:
(a) a tRNA;
(b) an aminoacyl-tRNA synthetase; and
(c) a tetrazine amino acid as described herein,
wherein the tRNA and aminoacyl-tRNA synthetase are an orthogonal tRNA/orthogonal aminoacyl-tRNA pair effective for incorporating the tetrazine amino acid into a protein to provide a tetrazine-labeled protein.

The kit can include additional components to facilitate in cellulo protein production using the tRNA, the aminoacyl-tRNA synthetase, and the tetrazine amino acid. Methods for use of tetrazine amino acids including their genetic encoding to provide a tetrazine-modified protein and bioorthogonal ligation using the tetrazine-modified protein are described in PCT/US2016/030469, entitled “Reagents and Methods for Bioorthogonal Labeling of Biomolecules in Living Cells”, expressly incorporated herein by reference in its entirety. These methods are applicable to the tetrazine amino acids of the disclosure.

The following describes methods for genetic encoding representative Tet ncAA,s described herein to provide a tetrazine-modified protein and bioorthogonal ligation using the tetrazine-modified protein.

Genetic Incorporation of Tet ncAAs in Proteins

To incorporate Tet ncAAs in the protein, a GCE machinery was developed to encode Tet ncAAs into the protein in response to TAG codon suppression with evolving orthogonal aaRS/tRNAcuA pair. The permissibility of developed synthetases for structurally parallel Tet ncAA derivatives was evaluated in 25 mL scale in presence and absence of 0.5 mM Tet ncAA.

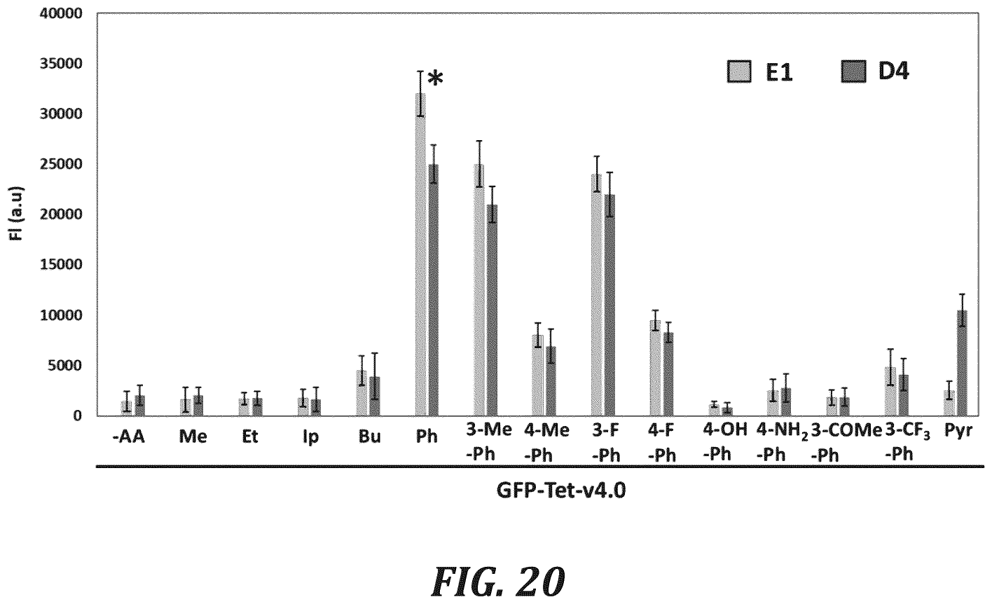

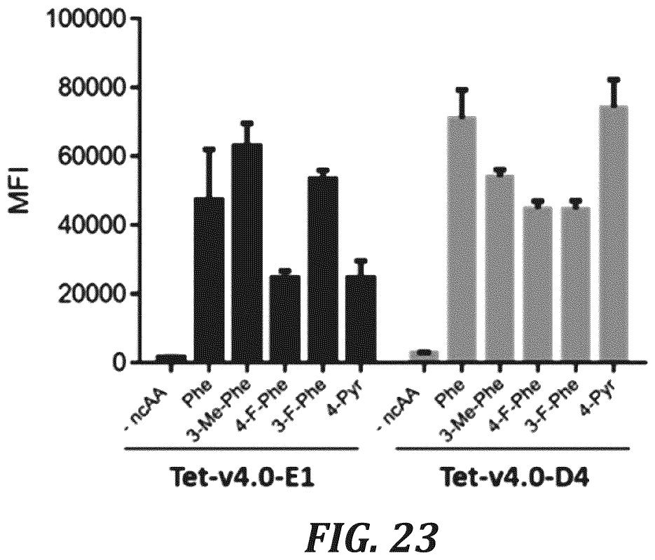

To incorporate Tet-v4.0 ncAAs both in prokaryotic and eukaryotic system, Mb-PylRS/tRNAcuA pairs D4 and E1 were evolved using Tet-v4.0Ph. Synthetases D4 and E1 were able to encode different substituents of phenyl derivatives Tet-v4.0Ph (3-Me, 3-F, 4-Me, 4-F, 3-CF₃) with moderate yields 5-50 mg/L. Although Tet-v4.0Ph-(4-OH, 4-NH₂, 3-COMe) were not permissive at all. Furthermore, only D4 was able to encode Tet-v4.0Pyr with moderate yield (about 35-40 mg/L) (see FIGS. 20 and 23). The expression efficiency also reveals that the meta-substituted Tet-v4.0Ph's are more permissive than para-substituted derivatives because of Pyl-RS active-sites steric accommodation.

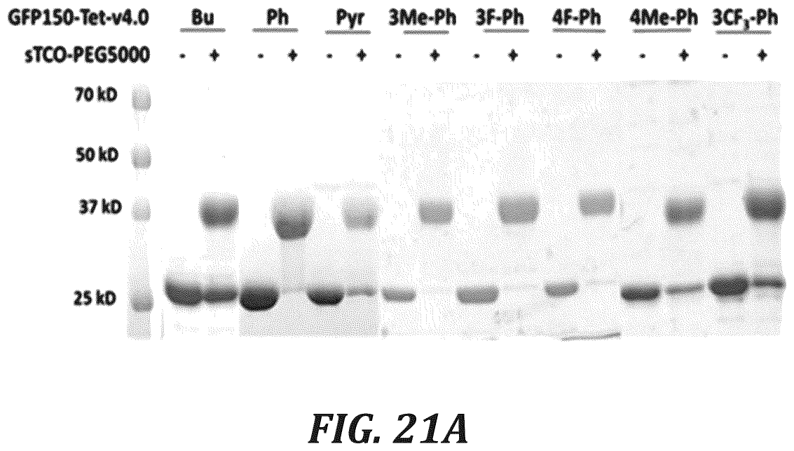

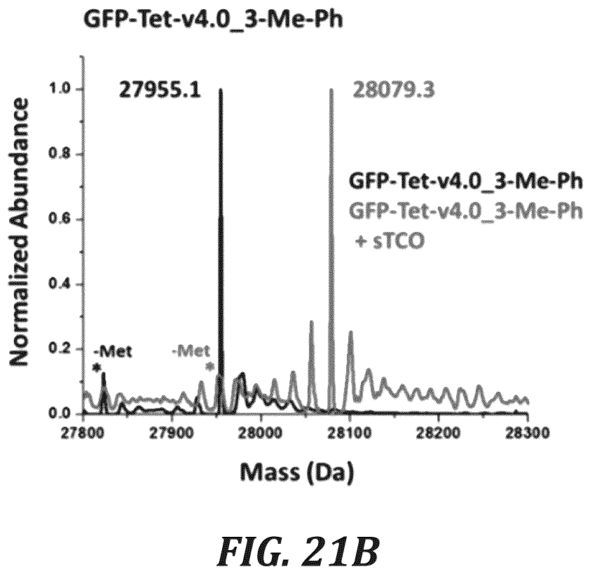

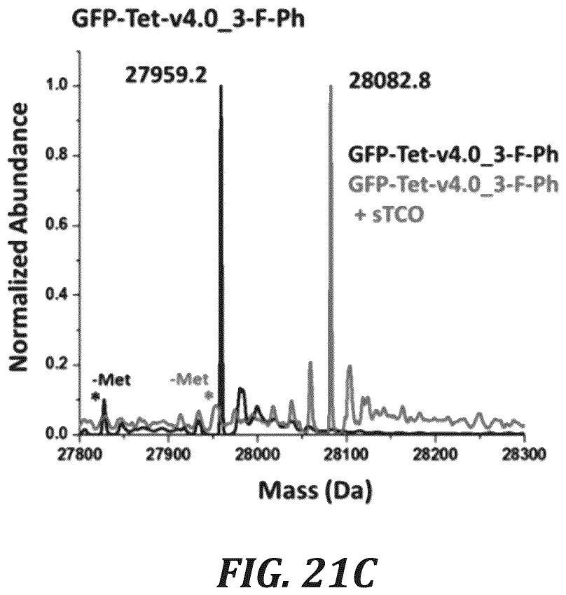

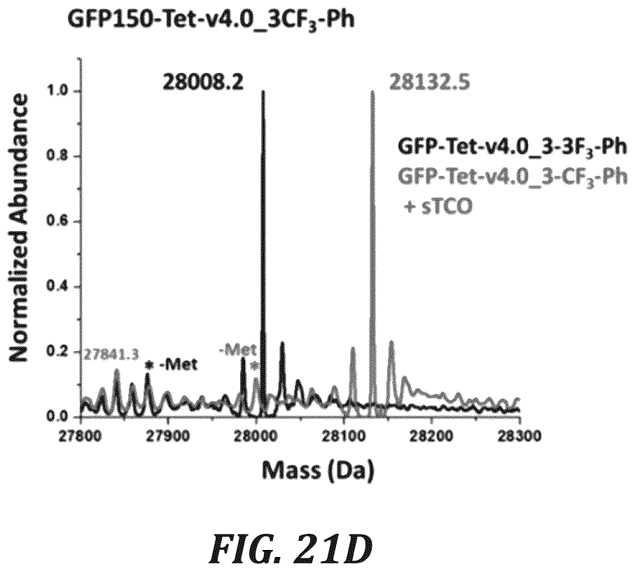

The incorporation efficiency and fidelity of the selected synthetases to the developed Tet-v4.0 ncAAs were verified by mass spectra analysis. The GFP150-Tet-v4.0Ph's (3-Me, 3-F, 3-CF₃) displayed a single major peak at higher mass compared to wt-GFP verified the asparagine residue at wt-GFP150 were replaced by single Tet-v4.0Ph ncAA's through amber codon suppression (FIG. 21B-21D). The high-fidelity attributes of Tet-v4.0 synthetases prevent near cognate suppression, however, the GFP-Tet-v4.0 3-CF₃-Ph shows a small amount of near-cognate suppression due to lower incorporation efficiency (FIG. 21D). Therefore, near-cognate suppression can be minimized with evolving an efficient next generation synthetases for Tet ncAA incorporation.

Tet ncAA Stability and Reactivity Inside Protein

To achieve clean and quantitative Tet-protein labeling, stability of incorporated Tet ncAA is essential. Recombinant protein production through biosynthetic pathway requires long time incubation of Tet-ncAA into the expression media. Whereas the reactive tetrazine scaffolds have a tendency to lose their reactivity either by reversible reduction or cross-reactivity with biological nucleophiles, tetrazine stability can be tuned by attaching substituents with different steric and electronic properties. The inherent stability-reactivity balance of Tet ncAA, which is inversely proportional to each other, is highly desirable for in-cell protein labeling.

The stability and reaction efficiency of incorporated Tet ncAAs in proteins was investigated. The Tet-proteins were stored at 4° C. in 50 mM PBS (pH 7.1) for two days after protein expressions (32 hrs. incubation in AIM at 37° C.) and purification. The SDS-PAGE gel mobility shift assay and mass spectrometry analysis of the incorporated Tet-v2.0, Tet-v3.0 and Tet-v4.0 derivatives confirmed their stability and reactivity with sTCO.

Mobility shift assay. Labeling efficiency and purity of Tet-containing proteins have been verified by reacting with 10 eqv. sTCO-PEG5k under PBS (pH 7.1) for 10 minutes at room temperature and examined the mobility shift of the reacted proteins in SDS-PAGE gel. The mobility shift assay helps to provide an insight into the reaction efficiency of Tet-containing proteins and also quantifies the amount of reactive Tet ncAA present in protein and measures if there is any misincorporation or Tet-degradation by showing unreactive protein. Here, almost all of GFP150-Tet have reacted, and a minimal unreacted band was observed for efficiently Tet-encoded proteins which measures the incorporated Tet ncAAs are stable and reacts quantitatively with sTCO. However, the less efficiently or poorly incorporated Tet-containing proteins are not able react quantitatively due to near-cognate suppression and resulted in considerable amount of unreacted proteins was observed for Tet-v4.0Ph (4-Me, 3-CF₃, Pyr) and Tet-4.0Bu (FIG. 21A).

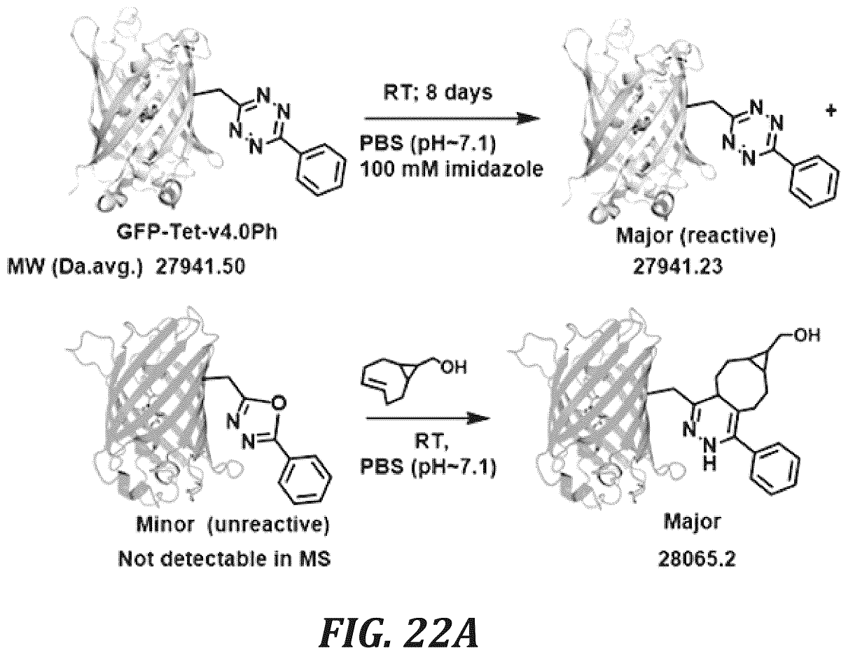

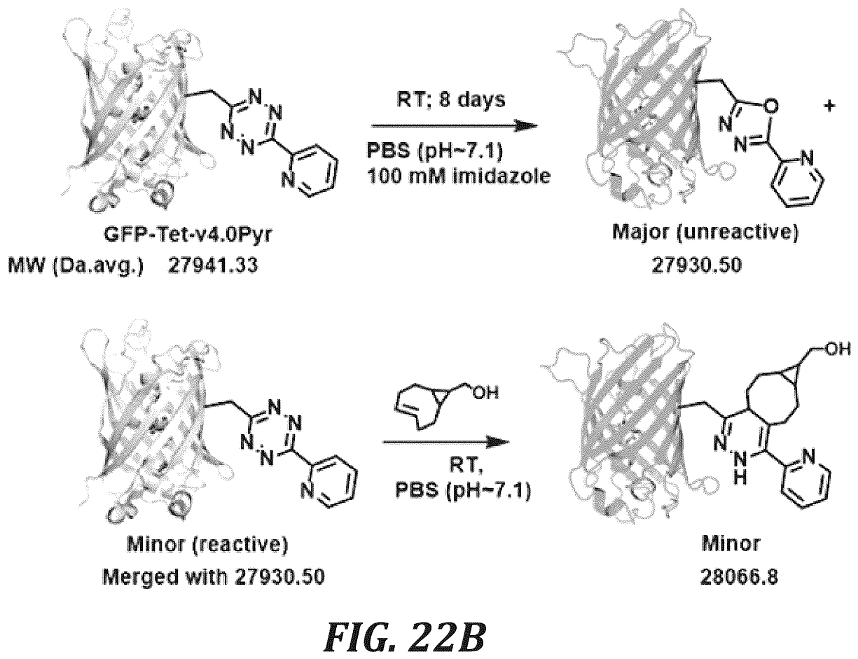

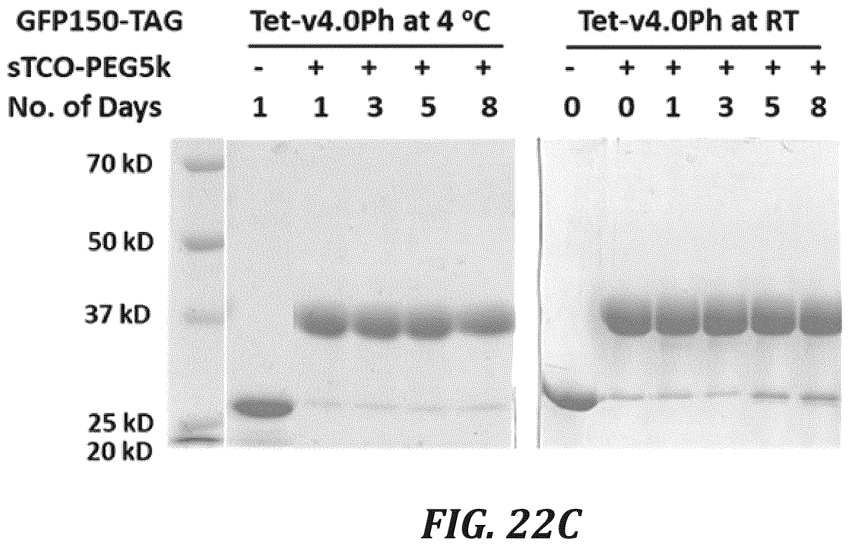

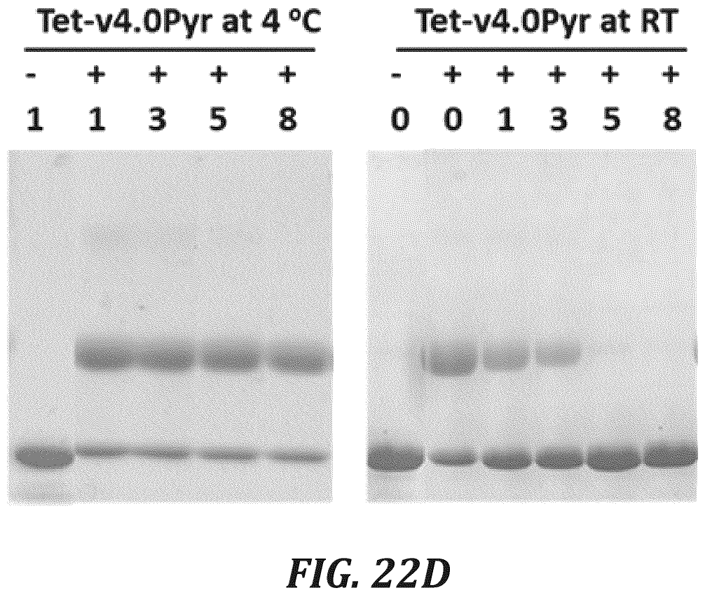

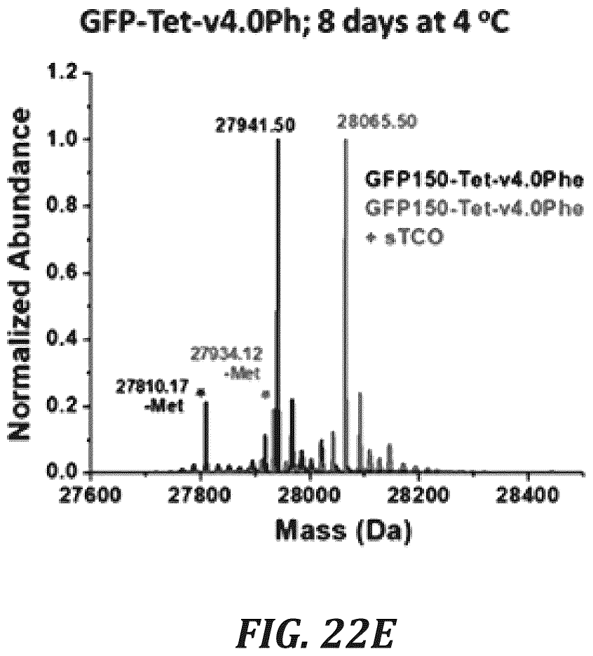

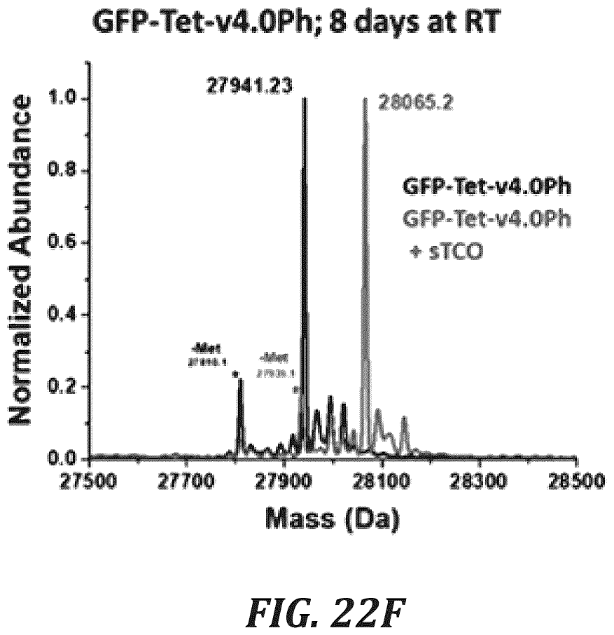

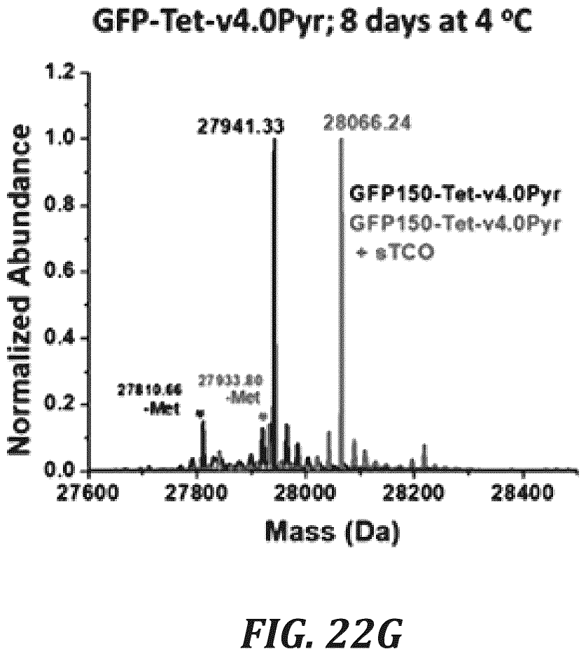

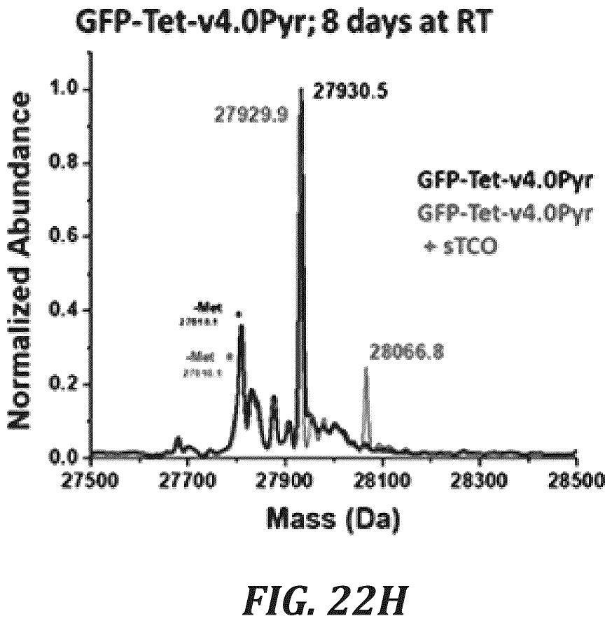

In addition, to assess their stability and reactivity more rigorously, GFP-Tet-v4.0 (Ph, 3-Me-Ph, 3-F-Ph and Pyr) were incubated at 4° C. and room temperature (RT) under 50 mM PBS (pH 7.1) with 100 mM imidazole and studied their reaction efficiency for another 8 days. The reactivity was observed to remain the same at 4° C., whereas at RT efficient labeling up to 3 days was shown followed by minimal degradation. However, the electron withdrawing pyridyl attached GFP-Tet-4.0Pyr degraded with time and no reaction was observed after 3 days due to loss of tetrazine functionality at RT (FIGS. 22A-22D).

Mass spectrometry. The site-specific labelling reaction between Tet ncAA and sTCO is quantitative inside protein was confirmed by ESI-Q-TOF mass spectrometry. Previously, a single major peak has been observed in mass spectra corresponding to single site Tet-incorporation into GFP150. Therefore, to study their reaction efficiency, the GFP-Tet proteins were exposed with 10 eqv. sTCO for 10 minutes under PBS (pH 7.1) at room temperature, then desalted the sample for mass spectrometry. As a result, the major single peaks were completely shifted to the 124.2 Da higher masses for the addition of sTCO with loss of N₂ and verified the Tet-sTCO adduct formation. The peak at 27941 Da corresponds to natural amino acids incorporation remains unreactive (FIGS. 22E-22G). No other peak has been detected for sTCO exposed samples which verified that the GFP-Tet reacts with sTCO quantitatively without involving side reactions.

However, at RT, the GFP-Tet-v3.0/4.0Pyr showed a major peak which is 11 Da unit less than expected mass and incubation with sTCO exhibited a small portion was reacted where the major mass remains unreactive (FIGS. 22E and 22F). The MS data for Tet-v4.0Pyr suggested that the major mass at 28006.5/27930.5 Da avg. corresponds to tetrazine degradation which was converted to its oxadiazole derivative and is therefore not reactive to the sTCO (FIGS. 22G and 22H). All above findings demonstrated that the methyl, ethyl, isopropyl, butyl, phenyl and substituted phenyl derivatives of Tet ncAA are stable and reactive inside protein however, the Pyr-Tet-ncAA degrades under harsh conditions.

Tet ncAA Kinetics with sTCO Inside Protein and their Limits

In application of labeling reaction under biological system, a fast kinetics (k₂>10⁴ M⁻¹s⁻¹) is necessary to compete with biological reaction. A high-speed labeling approach can reduce the labeling time and able to react at low concentrations of labels which is beneficial for low abundant protein labeling. Considering genetically encoded Tet ncAA based IEDAA reaction is under the mid-range of reported conjugation reaction as the fastest Tet-amino acids are less stable in aqueous system. While there are many factors such as electronic properties, steric hindrance of the substituents can tune the reactivity and varying the Tet-attached substituents significantly impacts on balancing the reaction rate and stability of Tet-amino acids. In general, tetrazine reactivity is enhanced by attaching electron-withdrawing substituents. However, doing so renders them more electrophilic and susceptible to nucleophilic attack and stimulating self-degradation. Electron-donating substituents slow reaction rate but improve stability. Therefore, the aqueous stability vs. fast reactivity needs to be optimized to develop an ideal system for tetrazine based efficient protein labeling both in vitro and in cell.

As described herein, Tet-v4.0 ncAA reactivity inside the protein varies by attaching different functional groups. The butyl, phenyl, tolyl, fluoro-phenyl and trifluoromethyl-phenyl attached Tet-v4.0 (Bu, Ph, CH₃-Ph, F-Ph, CF₃-Ph) ncAA derivatives exhibited a wide range of super-fast kinetics (k₂=21×10⁴ M⁻¹s⁻¹→62×10⁴ M⁻¹s⁻¹) with excellent stability. The Tet-pyridyl contained GFP-Tet-v4.0Pyr shows an ultra-fast kinetics (k₂=120×10⁴ M⁻¹s⁻¹) with slow degradation at RT.

Tet nCAA—sTCO Product Linkage Stability

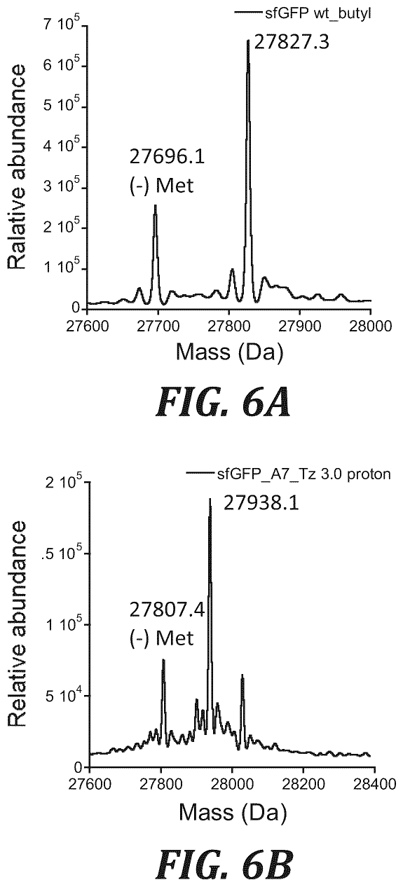

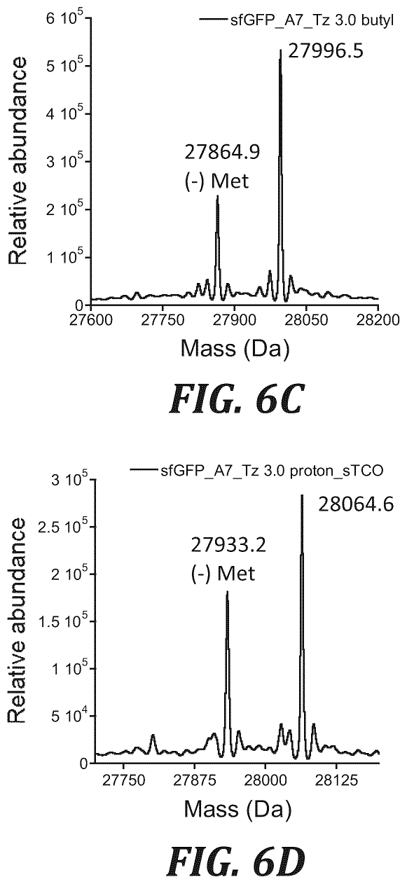

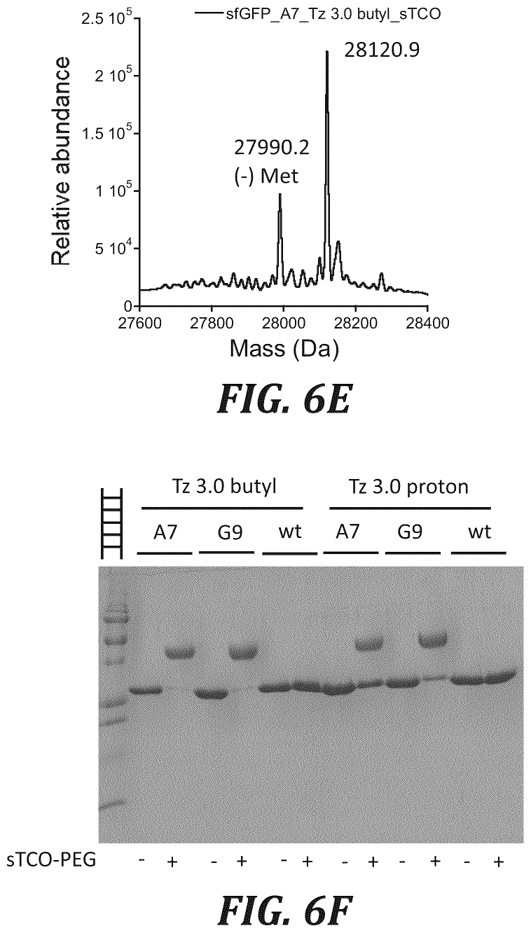

A stable linkage between the complementary reagents of ligation reaction is essential for labeling study. The stability and biocompatibility of the tetrazine-sTCO adduct under physiological condition is proven for various applications in different research areas. While both reagents sTCO and tetrazine are sensitive to acidic and basic conditions, respectively, the conjugated product 1,4-dihydropyridazine formed by carbon-carbon bond formation is stable and inert to biological milieu. Sometimes the conjugated product 1,4-dihydropyridazine aromatize to stable pyridazine for electron rich tetrazines under ambient conditions. Tetrazine-sTCO adduct stability inside protein was tested by SDS-page gel mobility shift assay and MS analysis. In SDS-PAGE gel, no additional mass shift was observed for GFP-Tet-v4.0-sTCO-PEG5k conjugated product after 8 days incubation in aqueous system (FIG. 6A). In addition, the GFP-Tet4.0Ph reacted with 10 eqv. sTCO in PBS and used for mass analysis after 8 days. The MS data shows a single peak due to the addition sTCO and loss of nitrogen without generating any degradation products (FIG. 6D).

### Eukaryotic Protein Labeling

Eukaryotic protein labeling under cellular environments is an essential tool to study protein dynamics, functions and localization in mammalian cells. A eukaryotic compatible GCE system for Tet ncAA incorporation is described herein and the toxicity and optimal concentration evaluated for effective protein expression in mammalian cells. HEK293T cells are healthy with 0.3 mM Tet ncAA and cell viability was compromised above 0.3 mM tetrazine amino acid and efficient protein production was observed at concentration level of 30 to 300 μM. Suppression efficiency was shown to increase about 30-40% after the addition of a nuclear export sequences (NES) to the aaRS. Thus, the genetically incorporated Tet-v3.0Bu by eukaryotic orthogonal pyrrolysyl-tRNA synthetase PylRS/tRNAcuA pairs shows high stability inside HEK293T cells expressed proteins and enables reaction with sTCO reagents without any detectable degradation products.

Tet4.0 ncAAs exhibited super-fast reactivity (k₂=2−120×10⁵ M⁻¹s⁻¹) with sTCO reagents and good stability inside proteins. Tet-v4.0Ph and Tet-v4.0Pyr have been shown to were encoded into HEK293T cells expressed proteins and their stability and reactivity verified by MS analysis and SDS-PAGE mobility shift assay upon reaction with sTCO-OH and sTCO-PEG5k.

To access newly developed Tet4.0 derivatives into mammalian cells, an optimized eukaryotic expression system was used and their stability and reaction efficiency were validated inside living cells. The permissibility of Tet-v4.0 derivatives into eukaryotic protein was tested with an evolved synthetase Tet-v4.0 E1 and D4 co-transfected with the reporter plasmid pAcBac1-sfGFP-TAG150 into the HEK293T cells and supplemented with 100 μM each of five different Tet-v4.0 (Ph, 3-Me-Ph, 3-F-Ph, 4-F-Ph, and Pyr) derivatives side-by-side. Across all cells suppression efficiencies were assessed using flow cytometry and observed that, as similar to E. coli expression, Tet-v4.0 E1 and D4 both are enable to encode Tet4.0 derivatives into GFP-TAG150 where the E1 was more efficient for 3-substituted Tet-v4.0Ph and the D4 incorporated Tet4.0Pyr relatively high yield.

To validate the small Tet-v4.0 derivatives are stable and enable for live cell labeling, HEK293T cells expressed GFP150-Tet4.0 derivatives were allowed to react with cell permeable fluorescent dye sTCO-JF646 by adding 100 nM into the cultured cell and incubated for one hour. The labeled cells were tracked by measuring GFP and JF-646 fluorescence using flow cytometry. The flow cytometry 2D-plots show all expressed cells are shifted upward that measures the expressed GFP contained Tet-v4.0 and labeled with sTCO-JF646 with minimal non-specific labeling. However, the non-specific labeling can be minimized by reducing the labeling time and probe concentration with increasing the labeling reaction rate through altering the Tet-amino acid reactivity. Remarkably, it was observed that the GFP-Tet-4.0_3-F-Ph and GFP-Tet-4.0Pyr labelled cells shifted-out diagonally as compared to other three Tet-v4.0 derivatives because of higher reactivity. These results demonstrate that compatibility and labeling ability of Tet-v4.0 into eukaryotic protein. It has also revealed that though the expression levels were considerably low but the reactivity of Tet-v4.0 inside protein is high enough for the labeling at the typically low protein concentrations (nM—low-μM) in mammalian cells.

The Tet-v4.0 ncAAs described herein (Tet-v3.0_4-F-Ph and Tet-v4.0 (-Ph, -3-Me-Ph, -4-Me-Ph, -3-F-Ph, -4-Me-Ph, -3-CF₃-Ph) maintain fast-kinetics and stability balance inside protein and accessible for both prokaryotic and eukaryotic systems.

Site-Directed Spin-Labeling of Proteins with Spin-Labeled Tet ncAAs

Site-directed spin-labeling (SDSL) in combination with double electron-electron resonance (DEER) spectroscopy has emerged as a powerful technique for determining both the structural states and the conformational equilibria of biomacromolecules. DEER combined with in situ SDSL in live cells is challenging because current bioorthogonal labeling approaches are too slow to allow for complete labeling with low concentrations of spin label prior to loss of signal from cellular reduction. As described herein, this limitation is overcome by genetically encoding tetrazine-bearing non-canonical amino acids (e.g., Tet-v4.0 ncAAs) at multiple sites in proteins expressed in E. coli and in human HEK293T cells. Specific and quantitative spin-labeling of Tet-v4.0-containing proteins was achieved by developing a series of strained trans-cyclooctene (sTCO)-functionalized nitroxides, including a gem-diethyl-substituted nitroxide with enhanced stability in cells, with rate constants that can exceed 10⁶ M⁻¹s⁻¹. The remarkable speed of the Tet-v4.0/sTCO reaction allowed efficient spin-labeling of proteins in live cells within minutes, requiring only sub-micromolar, medium concentrations of sTCO-nitroxide. DEER recorded from intact cells revealed distance distributions in agreement with those measured from proteins purified and labeled in vitro. Furthermore, DEER was able to resolve the maltose-dependent conformational change of Tet-v4.0-incorporated and spin-labeled MBP in vitro and support assignment of the conformational state of an MBP mutant within HEK293T cells.

In one aspect, the present disclosure provides a method for spin labeling a protein or functional protein fragment, comprising contacting a tetrazine-modified protein or a tetrazine-modified functional protein fragment with a cyclic alkene-spin label to provide a protein or functional protein fragment having a spin label covalently coupled thereto, wherein the tetrazine-modified protein or the tetrazine-modified functional protein fragment has been genetically encoded to include a tetrazine amino acid residue at a predetermined amino acid site.

As used herein, the term “genetically encoded” refers to genetic code expansion (GCE) resulting in the introduction of a non-canonical amino acid into a protein or functional protein fragment.

The terms “tetrazine-modified protein” and “tetrazine-modified functional protein fragment” refer to proteins and functional protein fragments, respectively, that have been genetically encoded to include a tetrazine amino acid residue.

The term “tetrazine amino acid” refers to a non-canonical amino acid that includes a tetrazine moiety capable of reaction with a cyclic alkene (e.g., trans-cyclooctene) via a strain-promoted inverse electron demand Diels-Alder (IEDDA) cycloaddition reaction.

The term “cyclic alkene-spin label” refers to a cyclic alkene that includes a spin label covalently linked to the cyclic alkene. Suitable cyclic alkenes include those that capable of reacting with the tetrazine moiety of a tetrazine-modified protein or a tetrazine-modified functional protein fragment resulting in covalent attachment of the spin label to the tetrazine-modified protein or a tetrazine-modified functional protein fragment via a strain-promoted inverse electron demand Diels-Alder (IEDDA) cycloaddition reaction between the tetrazine moiety and the cyclic alkene. Representative cyclic alkene-spin labels include trans-cyclooctene-spin labels. Useful spin labels include nitroxides, such as TEMPO nitroxide (tM6) and 5-membered pyrroline/pyrrolidine (PROXYL) nitroxides (tM5 and tE5).

In the method, the protein or functional protein fragment having a spin label covalently coupled thereto is a tetrazine-modified protein or the tetrazine-modified functional protein fragment that has been genetically encoded to include a tetrazine amino acid residue at a predetermined amino acid site.

As described below, in certain embodiments, the tetrazine amino acid residue is derived from a tetrazine amino acid having the formulae (I), (II), or (III):

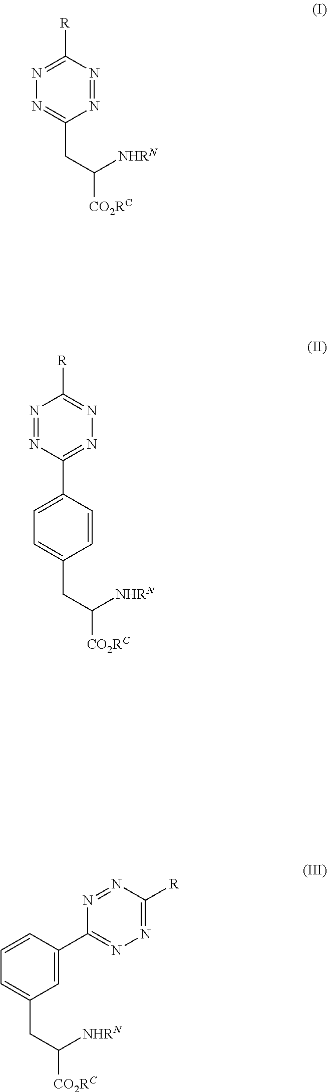

or a stereoisomer or salt thereof.

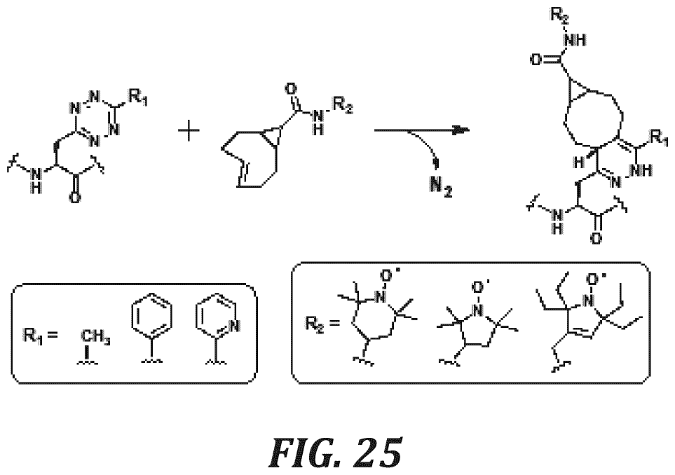

FIG. 25 illustrates the spin-labeling methods described herein using representative Tet-v4.0 ncAAs (Tet-v4.0 methyl, phenyl, and 2-pyridyl).

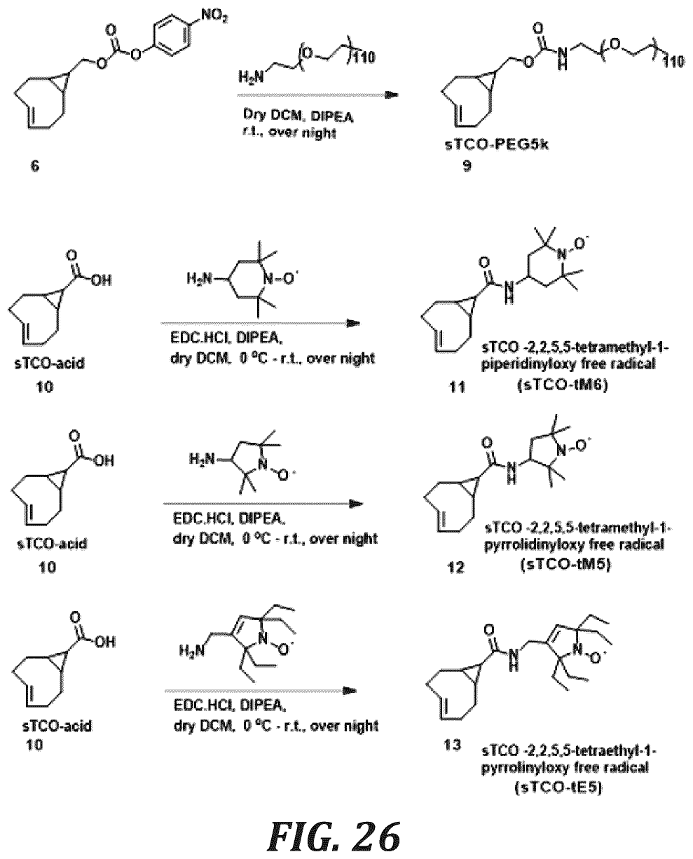

FIG. 26 illustrates the synthesis of sTCO-PEG5k and sTCO-tM6, sTCO-tM5, and sTCO-tE5 spin labels.

In another aspect, the disclosure provides a spin-labeled protein or functional protein fragment, comprising a protein or a functional protein fragment having at least one tetrazine amino acid residue, wherein the tetrazine amino acid residue is incorporated into the protein or the functional protein fragment at a predetermined amino acid site by genetic encoding of the protein or the functional protein fragment using a tetrazine amino acid, and wherein the spin label is attached to the protein or the functional protein fragment by covalent coupling to the tetrazine amino acid residue.

To facilitate genetic encoding of the tetrazines, an orthogonal aminoacyl tRNA synthetase (RS)/tRNA_{CUA} pair capable of site-specifically incorporating Tet4 into proteins using both prokaryotic and eukaryotic expression systems were evolved. The resulting Tet4-incorporated proteins were found to react about 2-15-fold faster with sTCO reagents than previously reported tetrazine ncAAs, generating the first site-specific bioorthogonal protein labeling reaction with rates exceeding 10⁶ M⁻¹s⁻¹.

Synthetase Selection for GCE with Tet-v4.0 ncAAs

To engineer a system for the genetic incorporation of Tet4 compatible with both prokaryotic and eukaryotic expression systems, the pyrrolysyl-tRNAcuA/amino-acyl-tRNA synthetase pair (Pyl-tRNA/RS) from Methanosarcina barkeri (Mb) was evaluated. Two Mb-Pyl-RS libraries with 5 sites mutated to all 20 amino acids (3×10⁶ total variants) were evaluated for the ability to accommodate Tet4-Me and Tet4-Ph. Standard life/death selection procedures involving two rounds of positive selection in the presence of 0.5 mM Tet4 ncAA and negative selection in the absence of ncAA were performed. The surviving synthetase variants were further evaluated for the ability to suppress an amber codon substituted at residue N150 of green fluorescent protein (GFP150-TAG). In total, four unique synthetases were identified that could express full-length GFP in the presence, but not in the absence, of Tet4-Ph, with two synthetase variants, D4 and E1, demonstrating high-fidelity incorporation and good yields for GFP150-TAG (about 55-60 mg/L)

To incorporate Tet4-Pyr, permissibility tests were performed using the same four unique synthetases identified in the Tet4-Ph screen and found that only synthetase D4 was able to selectively incorporate Tet4-Pyr into GFP150-TAG with reasonable yields (about 40 mg/L). The site-specific incorporation efficiency and fidelity of the E1 synthetase (for Tet4-Ph) and D4 synthetase (for Tet4-Pyr) were further verified by mass spectrometry of C-terminally 6× His-tagged GFP150-TAG constructs purified from E. coli. Deconvolutional analysis of the ESI-Q-TOF mass spectra yielded the expected masses confirming substitution of the native asparagine with Tet4-Ph or Tet4-Pyr, respectively. These results collectively demonstrate that evolved synthetases E1 and D4 can efficiently encode Tet4-Ph and Tet4-Pyr ncAAs, respectively, in E. coli.

### Reactivity of Protein-Encoded Tet-v4.0

To test the accessibility and reactivity of Tet4 ncAAs toward sTCO reagents once incorporated into proteins, purified samples of GFP150-Tet4-Ph/Pyr were incubated with sTCO-OH and the labeling reaction monitored by mass spectrometry. Analysis of the ESI-Q-TOF data revealed apparently quantitative labeling as evidenced by mass increases of 124 Da for both GFP150-Tet4 proteins, consistent with the expected sTCO-OH cycloaddition product and concomitant loss of N₂. Labeling of GFP-Tet4-Ph/Pyr was also verified using a general SDS-PAGE mobility shift assay based on reaction with PEGylated sTCO reagents (sTCO-PEG5k/10k). GFP150-Tet4-Ph displayed a near complete mass shift of about 5 kDa upon 10-minute incubation with 10-fold excess sTCO-PEG5k, indicating that virtually all protein contained the tetrazine and was quantitatively conjugated with sTCO-PEG5k. A similar shift was observed for GFP150-Tet4-Pyr but with a small percentage of unreacted protein remaining, suggesting that some full-length GFP either did not contain Tet4-Pyr at residue 150, most likely from insertion of a canonical amino acid by way of near-cognate suppression, or were otherwise unreactive, for example, due to tetrazine degradation. However, unreacted or degraded proteins were not detected by mass spectrometry, suggesting their abundance is low.

DEER studies on proteins lacking intrinsic spin centers generally require the introduction of spin labels at two sites in the primary sequence. To verify that successful encoding of Tet4 at two sites, GFP constructs containing dual amber codon (TAG) substitutions at residues N150 and L222 were generated. Mass spectral analysis of purified GFP150/222-Tet4-Ph confirmed the correct mass for doubly Tet4-Ph-incorporated GFP at residues 150 and 222, and subsequent reaction with sTCO-tE5 spin label revealed a mass increase of 690 Da, corresponding to the addition of two sTCO-tE5 spin labels minus two equivalents of molecular nitrogen. Dual labeling of GFP150/222-Tet4-Ph was additionally confirmed by SDS-PAGE gel-band shift after reaction with sTCO-PEG5k. Together these experiments demonstrate near-quantitative reactivity of singly and doubly Tet4-incorporated proteins.

The IEDDA reaction rates of Tet4 ncAAs incorporated at residue 150 of GFP were determined. Tetrazine amino acids are known to partially quench GFP fluorescence when incorporated near the protein chromophore. Subsequent reaction of the tetrazine to form the 1,4-dihydropyridazine product removes this quenching effect and provides a sensitive measure of the reaction progress. Time-dependent dequenching (increase in fluorescence) of purified GFP150-Tet4-Ph/Pyr upon reaction with sTCO-OH was measured. Plots of pseudo-first order rate constants (k′) against sTCO-OH concentration revealed 2^{nd}-order rate constants (k₂) of 2.2×10⁵ and 1.2×10⁶ M⁻¹s⁻¹ for phenyl and pyridyl variants of Tet4, respectively. These represent the fastest reaction rates reported to date for genetically encoded tetrazine amino acids. Rate constants (k₂) for all three sTCO-nitroxides in reaction with GFP150-Tet4-Ph were measured. The observed reaction rates for sTCO-tM6, sTCO-tM5, and sTCO-tE5 were about 2-4×10⁵ M⁻¹s⁻¹, consistent with rates measured for sTCO-OH. The magnitude of these reaction rates reveals that spin-labeling is complete in seconds to minutes, even at sub-micromolar concentrations, and suggest that rapid and complete labeling of dilute protein solutions are possible using stoichiometric amounts of sTCO-spin label.

### In Vitro EPR and DEER

To investigate the utility of Tet4 incorporation for site-directed spin-labeling, maltose binding protein (MBP) was chosen as a model. MBP undergoes a well-characterized clamshell closure that is stabilized by the binding of maltose and other maltodextrin ligands. The structures and energetics of this conformational change have been subject to extensive study using numerous biophysical techniques, including DEER, NMR, FRET, and X-ray crystallography. Four residues in the MBP sequence (5211, E278, K295, and E322) were selected for mutation either to cysteine, for labeling with the thiol-specific spin label MTSL, or to Tet4 ncAA for labeling with sTCO-spin labels. In addition, we generated double-mutants with site-pairs 211/295 and 278/322 were generated to probe the maltose-dependent conformational change of MBP using DEER. Representative Tet4-Ph ncAA was chosen for incorporation with synthetase Mb-PylRS E1 for use in the spin-labeling described herein.

Single and double cysteine and Tet4-Ph mutants of MBP were purified from E. coli and labeled with MTSL or sTCO-labels, respectively. Reaction of doubly Tet4-Ph-incorporated MBP constructs with sTCO-tE5 showed quantitative dual labeling by mass spectroscopic analysis. Likewise, reaction with sTCO-PEG10k showed two-site labeling efficiencies of 85-90% by SDS-PAGE mobility shift assay. In corroboration, labeling efficiencies of sTCO-nitroxides, as determined by double integration of the continuous-wave (CW) EPR spectra, were estimated to be ≥75% for all MBP-Tet4-Ph sites studied. CW EPR spectra recorded at room temperature revealed that the side chains of MBP-Tet4-Ph proteins labeled with sTCO-tM6 or -tM5 are significantly more rigid than are cysteines labeled with MTSL at equivalent residues, as evidenced by their larger spectral widths and correspondingly larger absolute-value first moments, [symbol] |ΔH| [symbol] . This reduced rotational mobility is not surprising given the relatively bulky macrocyclic sidechain of spin-labeled Tet4 and the small number of rotatable bonds. MBP-Tet4-Ph sites labeled with sTCO-tE5 display more mobile EPR spectra, similar to MTSL-labeled MBP.

The ability of doubly spin-labeled MBP-Tet4 constructs to report intramolecular distances and ligand-dependent conformational changes in MBP with DEER spectroscopy was examined. Crystal structures of apo and maltose-bound MBP indicate a decrease in C_{β}-C_{β} distance between residues S211 and K295 of about 8 Å upon binding of maltose and closure of the clamshell. Conversely, the C_{β}-C_{β} distance between E278 and E322, located on the opposite surface of MBP (backside of the clamshell), increases slightly (about 3 Å) in the maltose-bound structure. Indeed, 4-pulse DEER on MTSL-labeled MBP211C/295C clearly revealed the expected decrease in intramolecular distance in the presence of 1 mM maltose relative to the maltose-free sample. Likewise, DEER revealed the small increase in distance expected upon maltose binding for MBP278C/322C labeled with MTSL. To test whether the Tet4-based spin-labeling system was capable of discerning the maltose-driven clamshell conformational change, DEER was recorded on doubly Tet4-Ph-encoded MBP constructs spin-labeled with sTCO-nitroxides, both in the presence and in the absence of maltose. Distance distributions obtained for apo and maltose-bound MBP211/295-Tet4-Ph labeled with sTCO-tE5 were in reasonable agreement with predictions from in silico rotameric modeling and displayed significantly broader distributions compared to the MTSL-labeled constructs. Nevertheless, a clear shift toward shorter inter-spin distances was observed in the sample containing 5 mM maltose, consistent with the maltose-induced closure of MBP.

Surprisingly, DEER on sTCO-tE5-labeled MBP278/322-Tet4-Ph revealed a pronounced bimodal distance distribution both in the presence and absence of maltose, with only one distance mode displaying the expected increase in inter-spin distance in response to maltose. In silico spin label modeling using apo and maltose-bound MBP structures suggested that this multi-modality stems from restricted conformational sampling of the spin labels owing to the relatively bulky and rigid macrocyclic adduct being close to the protein surface. Rotameric models suggested that steric clashes with neighboring residues may “trap” spin labels in distinct clusters of closely related conformations, giving rise to multimodal distance distributions. Indeed, multimodal distance distributions were also observed for tM6- and tM5-labeled MBP-Tet4-Ph constructs. Altogether, these results show that DEER on in vitro spin-labeled Tet4-encoded proteins can reveal ligand-induced conformational changes giving rise to DEER distance changes as small as a few ångstroms; however, multimodal DEER distributions, likely arising from spin label rotameric restrictions, may be problematic at some protein sites and potentially complicate structural interpretation.

### Tet-v4.0 Encoding and Spin-Labeling in Mammalian Cells

The compatibility of the Tet4 system with mammalian cells was explored. Specifically, the ability of Tet4 to be incorporated into proteins expressed in HEK293T cells, an immortalized human embryonic kidney cell line, was explored. Tet4-Ph and Tet4-Pyr ncAAs were well-tolerated by HEK293T cells when supplemented into the growth medium at concentrations up to 0.3 mM, whereas higher concentrations resulted in cytotoxicity. Genes for Tet4 E1 and D4 synthetases were cloned with a terminal nuclear export sequence into the eukaryotic expression Pyl-tRNA/RS vector pAcBac1. Pyl-tRNA/RS pairs were then co-transfected with pAcBac1-GFP150-TAG into HEK293T cells and incubated either in the absence of ncAA, or with various concentrations of Tet4 ncAAs, and suppression efficiencies were assessed using flow cytometry. Tet4-Ph and Tet4-Pyr were incorporated into GFP about 50% as well as the Tet-v3.0 system, Tet3-Bu. As with Tet4 incorporation into E. coli expressed proteins, the highest suppression efficiencies in HEK293T cells were obtained with Mb-PylRS synthetase E1 and Tet4-Ph ncAA. Selective incorporation and reactivity of GFP150-Tet4 proteins were verified in whole-cell HEK293T lysates using sTCO-PEG5k-induced SDS-PAGE mobility shifts, and in purified proteins by mass spectrometry.

In situ spin-labeling Tet4-incorporated proteins in living HEK293T cells was explored. The reactivity of Tet4-encoded proteins in HEK293T cells was tested by exposing cells expressing GFP150-Tet4-Ph or Tet4-Pyr to the cell-permeable fluorophore sTCO-JF646. Cells were then washed, lysed, and analyzed by SDS-PAGE with fluorescence detection of both GFP and JF646. These results verified conjugation of JF646 exclusively at GFP, with undetectable labeling of off-target proteins. The reactivity of GFP150-Tet4 in HEK293T cells was further explored by exposing cultured cells to sTCO-JF646 at two different concentrations, 100 nM and 1 μM, for 30 minutes before quantifying GFP and JF646 fluorescence of individual cells using flow cytometry. The strong linear relationship between GFP and JF646 signals indicate that efficient, GFP-specific in-cell labeling occurred at 100 nM sTCO-JF646 with very little non-specific labeling. At 1 μM sTCO-JF646, efficient GFP labeling was also achieved; however, significant background JF646 fluorescence was observed which suggests off-target association of the label with other cellular components. These experiments demonstrate that the fast reaction kinetics of Tet4-encoded proteins with sTCO reagents permits stoichiometric labeling in live eukaryotic cells, even at low concentrations of both label and protein.

The in-cell labeling properties of sTCO-JF646 were exploited to assess the permeability and reactivity of the sTCO-spin labels in live HEK293T cells using a pulse-chase assay. HEK293T cells expressing MBP322-Tet4-Ph were incubated with various concentrations of the reduction-resistant spin label sTCO-tE5 for 30 minutes, after which the reaction was quenched with excess sTCO-JF646 (0.5 μM, 30 min, r.t.). Cells were then lysed and lysates analyzed using SDS-PAGE with fluorescence detection. 100 nM sTCO-tE5 reduced the JF646 fluorescence 2-fold, indicating that roughly half of the available Tet4-Ph-incorporated MBP had been spin-labeled, whereas application of 250 nM sTCO-tE5 or higher led to complete spin-labeling of all available MBP-Tet4 in the cell.

To further examine the time course of sTCO-tE5 labeling of HEK293T cells expressing GFP150-Tet4-Ph, the cells were incubated in culture medium containing 1 μM sTCO-tE5 while imaging GFP fluorescence with epifluorescence microscopy. As sTCO-tE5 entered cells and reacted with GFP150-Tet4-Ph, the quenching effect of Tet4 on GFP fluorescence was relieved and an increase in fluorescence intensity was observed. Under these conditions, the in-cell spin-labeling reaction appeared complete within 2 minutes. It should be noted that the labeling kinetics observed in this experiment are a product not only of the concentration-dependent IEDDA reaction rate, but also of the perfusion and mixing times, as well as the time required for sTCO-tE5 to diffuse across the cell membrane. These experiments suggested that intracellular Tet4-incorporated proteins can be quantitatively spin-labeled with our sTCO-nitroxides, directly within living cells, in a matter of minutes, using sub-micromolar concentrations of spin-label.

### In-Cell EPR and DEER

To assess the possibility of detecting spin-labeled Tet4 proteins in mammalian cells by EPR, HEK293T cells expressing GFP150-Tet4-Ph were incubated in medium supplemented with 200 nM sTCO-tE5 for 10 minutes, after which the labeling medium was removed, the cells transferred into a quartz EPR tube, and CW EPR spectra of the pelleted cells recorded at ambient temperature. The EPR spectrum, recorded 1 hour after first exposure to spin label, revealed a slow-motional spectrum very similar to that of GFP150/222-Tet4-Ph purified from E. coli and spin-labeled with sTCO-tE5 in vitro. This EPR signal was significantly reduced in spin-labeled cells in which the plasmid encoding the PylRS/tRNA pair was omitted from the transfection or where expression was carried out in the absence of Tet4-Ph ncAA. Similar synthetase-dependent in-cell CW EPR signals were also obtained from sTCO-tE5-labeled MBP295-Tet4-Ph and MBP322-Tet4-Ph. Although greatly reduced, there was still a notable slow-motion EPR spectrum in spin-labeled cells grown in the presence of Tet4-Ph but lacking the plasmid encoding the PylRS/tRNA pair. This likely stems from residual free Tet4-Ph ncAA in the cells being conjugated with sTCO-tE5 and highlights the importance of thoroughly washing free ncAA from the cells prior to exposure to sTCO reagents. Together these results demonstrate the ability of the reduction-resistant nitroxide sTCO-tE5 to site-specifically label proteins harboring Tet4 within living mammalian cells, generating EPR signals that persist for at least 1 hour at ambient temperatures with minimal background labeling.

To test whether accurate intramolecular protein distances could be measured by DEER in mammalian cells using the bioorthogonal Tet4/sTCO-nitroxide system, GFP constructs were generated for both bacterial and mammalian cell expression containing dual amber codon (TAG) substitutions at residues N150 and L222. GFP was chosen for initial in-cell DEER experiments because it is a small, well-structured protein devoid of large-scale conformational changes, thus allowing interpretation of the distance distribution without interference from conformational heterogeneity contributed by protein dynamics. Indeed, 4-pulse DEER of doubly Tet4-Ph-incorporated GFP purified from E. coli and subsequently spin-labeled with sTCO-tE5 yielded a mono-modal distance distribution centered at about 25 Å, in agreement with the simulated distance distribution predicted by rotameric modeling. The large modulation depth (about 50%) observed in the DEER time-domain data is again indicative of highly efficient spin label conjugation at both Tet4 sites.

GFP150/222-Tet4-Ph was expressed in HEK293T cells. After incubation with 250 nM sTCO-tE5 for 10 minutes, cells were washed once with PBS buffer before loading into an EPR tube and snap-freezing in liquid nitrogen for DEER measurement. The echo modulation obtained for the in-cell sample was significantly smaller than for the in vitro-purified sample, indicating a high percentage of singly spin-labeled molecules that do not contribute to echo modulation. These lone spins likely result from several sources: (1) chemical reduction of the nitroxides in the cell will increase the proportion of proteins having only one or zero active spin centers; (2) proteins truncated at the second amber codon will produce peptides having only one Tet4-Ph side chain available for spin-labeling; and (3) spin labels either free in solution or conjugated to residual tRNA-loaded or unincorporated Tet4-Ph will also result in a non-modulated DEER echo, decreasing the experimental modulation depth. Nevertheless, the resulting distance distribution obtained from intact HEK293T cells is very similar to the distance distribution obtained from purified protein, with distances of maximum probability—24.4 Å (in vitro) and 25.5 Å (in-cell)—differing by 1.1 Å. The in-cell DEER distribution does, however, contain a lower probability peak centered around 50 Å that is not present in the in vitro distribution. Given that the time-domain data were only collected out to about 1.8 μs, probabilities in this distance range are often unreliable and are likely attributable to uncertainties in the DEER background separation.

Having shown that accurate distance distributions could be obtained in HEK293T cells with the Tet4/sTCO-tE5 system, Tet4 incorporation and in vivo spin-labeling was evaluated to determine the conformational state of a multi-state protein directly within the cellular environment. A mutation (W340A) within the maltose binding pocket of MBP, known to lower the affinity for maltodextrin ligands by about two orders of magnitude, was chosen for evaluation.

The in vitro DEER experiments on doubly sTCO-tE5-labeled MBP211/295 showed a clear shift in the distance distributions between apo and maltose-bound samples. Consequently, 211TAG and 295TAG mutations, along with W340A, were chosen for introduction into the mammalian MBP expression construct for in-cell DEER measurements. MBP211/295-TAG (W340A) was co-transfected with PylRS/tRNA E1 into HEK293T cells in the presence of 100 μM Tet4-Ph ncAA and the cells were spin-labeled with sTCO-tE5 in the same manner as described for the GFP DEER construct. CW EPR spectra were recorded from spin-labeled whole cells as a function of time. The spectra show a gradual decay of the nitroxide EPR signal over the course of a few hours, approximately 2-fold faster than was observed with free sTCO-tE5 spin label in diluted cell extract. The lineshape of the CW EPR spectra recorded from cells was nearly identical to that obtained from the same construct purified from E. coli or HEK293T cells and spin-labeled in vitro. The distance distribution obtained from DEER of the intact cells revealed a primary peak centered at about 39 Å, consistent with the apo (clamshell open) conformation of MBP measured from in vitro-purified protein, in agreement with the expected effect of the W340A mutation.

In one aspect, the disclosure provides a method for spin labeling a protein or functional protein fragment, comprising contacting a tetrazine-modified protein or a tetrazine-modified functional protein fragment with a cyclic alkene-spin label to provide a protein or functional protein fragment having a spin label covalently coupled thereto, wherein the tetrazine-modified protein or the tetrazine-modified functional protein fragment has been genetically encoded to include a tetrazine amino acid residue at a predetermined amino acid site.

In certain embodiments, the spin labeling method described herein is an in cellulo method (i.e., an in-cell labeling method).

In certain embodiments of the method, the tetrazine-modified protein or the tetrazine-modified functional protein fragment is an enzyme or functional fragment thereof, a binding protein or functional fragment thereof, or an antibody or functional fragment thereof.

In certain embodiments, the protein or functional protein fragment is covalently coupled to the spin label via reaction of the tetrazine moiety of the tetrazine-modified protein or the tetrazine-modified functional protein fragment with the cyclic alkene of the cyclic alkene-spin label.

In certain embodiments, the cyclic alkene-spin label is a trans-cyclooctene-spin label. In certain of these embodiments, the spin label is a nitroxide.

In certain embodiments, the tetrazine amino acid residue is derived from a tetrazine amino acid having the formulae (I), (II), or (III):

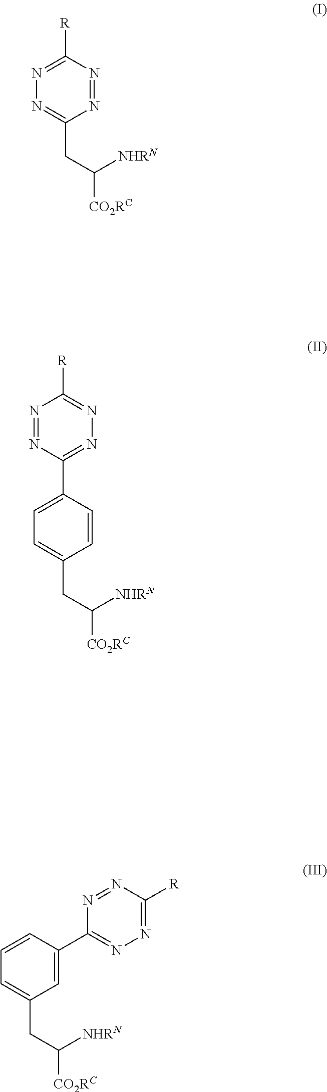

or a stereoisomer or salt thereof, wherein
R is selected from the group consisting of:
(a) hydrogen,
(b) a substituted or an unsubstituted C1-C6 alkyl group,
(c) a substituted or an unsubstituted phenyl group,
(d) a substituted or an unsubstituted heteroaryl group,
(e) a substituted or an unsubstituted heterocyclyl group,
(f) an amino C1-C6 alkyl group,
(g) a thio C1-C6 alkyl group, and
(h) a carboxylate group;
R^{C} is hydrogen, a counter ion, or a carboxyl protecting group; and
R^{N} is hydrogen or an amine protecting group.

Embodiments of the various R substituents are as described above.

In other embodiments, the tetrazine amino acid residue is derived from a tetrazine amino acid as described in PCT/US2016/030469, expressly incorporated herein by reference in its entirety.

In another aspect of the disclosure, a spin-labeled protein or functional protein fragment is provided. In certain embodiments, the spin-labeled protein or functional protein fragment, comprises a protein or a functional protein fragment having at least one tetrazine amino acid residue, wherein the tetrazine amino acid residue is incorporated into the protein or the functional protein fragment at a predetermined amino acid site by genetic encoding of the protein or the functional protein fragment using a tetrazine amino acid, and wherein the spin label is attached to the protein or the functional protein fragment by covalent coupling to the tetrazine amino acid residue.

As noted above, in certain embodiments, the tetrazine-modified protein or the tetrazine-modified functional protein fragment is an enzyme or functional fragment thereof, a binding protein or functional fragment thereof, or an antibody or functional fragment thereof; the protein or functional protein fragment is covalently coupled to the spin label via reaction of the tetrazine moiety of the tetrazine-modified protein or the tetrazine-modified functional protein fragment with the cyclic alkene of the cyclic alkene-spin label (e.g., a trans-cyclooctene-spin label, wherein the spin label is a nitroxide; and the tetrazine amino acid has one of the formulae noted above.

### Materials and Methods

### General Synthetic Methods

All purchased chemicals were used without further purification. 3-Amino-PROXYL was purchased from Toronto Research Chemicals and 3-aminomethyl-2,2,5,5-tetraethyl-1-pyrrolinyloxy free radical was synthesized according to the published procedure. Anhydrous dichloromethane (DCM) and dimethyl sulfoxide were used after overnight stirring with calcium hydride and distillation under argon atmosphere. Thin layer chromatography (TLC) was performed on silica 60F-254 plates. The TLC spots of alkene were identified by potassium permanganate staining. Flash chromatographic purification was performed using silica gel 60 (230-400 mesh size). ¹H NMR spectra were recorded on Bruker at 400 MHz and 700 MHz and ¹³C NMR spectra were recorded at 175 MHz. The chemical shifts were shown in ppm and are referenced to the residual non-deuterated solvent peak CDCl₃ (δ=7.26 in ¹H NMR, δ=77.23 in ¹³C NMR), CD₃OD (δ=3.31 in ¹H NMR, δ=49.2 in ¹³C NMR), d₆-DMSO (δ=2.5 in ¹H NMR, δ=39.5 in ¹³C NMR) as an internal standard. Splitting patterns of protons are designated as follows: s—singlet, d—doublet, t—triplet, q—quartet, m—multiplet, bs—broad singlet, dd—doublet of doublets.

Tetrazine amino acid (Tet4) rate constant measurements. The solutions of tetrazine amino acids (0.2 mM) and sTCO-OH (1.0-6.0 mM) were made in PBS (137 mM NaCl, 2.7 mM KCl, 10 mM Na₂HPO₄, 1.8 mM KH₂PO₄, pH 7.4) with 2% methanol to ensure solubility of Tet ncAAs. The measured loss of tetrazine absorbance at 270 nm was used to determine the reaction rate of Tet ncAAs. Pseudo-first order conditions were employed with sTCO-OH in 5- to 60-fold excess of tetrazine amino acids at 25° C. All measurements were performed in triplicate and the resulted decay curves were fit to a single exponential equation. The mean values of pseudo first order rate constants (k′) were plotted against different concentration of sTCO-OH to obtain second order rate constants (k₂) from the slope of the plot.

Selection of aminoacyl-tRNA synthetases for Tet4 incorporation. To incorporate Tet4-Me and Tet4-Ph, a library of all amino acids at 5 sites (Asn311, Cys313, Val366, Trp382, and Gly 386) from Methanosarcina barkeri (Mb) system was chosen for its ability to incorporate large aromatic amino acids. The synthetase (RS) library was encoded on a kanamycin (Kn) resistant plasmid (pBK, 3000 bp) under control of the constitutive Escherichia coli GlnRS promoter and terminator (pBK-RS library). The pBK-RS library was mutated at the above-mentioned sites with NNK codons corresponding to all 20 natural amino acids where N is A, C, G, or T and K is G or T. The pBK-RS library was transformed into DH10b cells containing a positive selection plasmid (pRep pylT) for “positive” selection steps and DH10b cells containing a negative selection plasmid (pYOBB2 pylT) for “negative” selection steps.

The positive selection plasmid, pRep pylT (10000 bp), encodes a mutant (Mb) pyrrolysyl-tRNAcuA, an amber codon-disrupted chloramphenicol acetyltransferase, an amber codon-disrupted T7 RNA polymerase, a T7 promoter controlled GFP gene, and the tetracycline (Tcn) resistance marker. The negative selection plasmid, pYOBB2 pylT (7000 bp), encodes the mutant pyrrolysyl-tRNAcuA, an amber codon-disrupted barnase under control of an arabinose promoter and rrnC terminator, and the chloramphenicol (Cm) resistance marker. pRep pylT electrocompetent cells and pYOBB2 pylT electrocompetent cells were made from DH10B cells carrying the respective plasmids and stored in 100 μL aliquots at −80° C.

Following the general selection procedure, positive and negative selections were carried out on the pBK-library. For positive selections, LB agar media plates containing 60 μg/mL chloramphenicol (Cm), 50 μg/mL kanamycin (Kn), 25 μg/mL tetracycline (Tcn) and 0.5 mM tetrazine amino acids were used. For negative selections, LB agar plates containing 25 μg/mL chloramphenicol (Cm), 50 μg/mL kanamycin (Kn) and 0.2% arabinose. The pBK-RS library was moved back and forth between positive and negative cell selections for two full rounds (positive 1, negative 1, positive 2, negative 2). The remaining pBK-RS library from the second negative selection transformed into DH10B cells containing the pALS to evaluate individual pBK-library members. The pALS plasmid contains the sfGFP reporter with a TAG codon at residue 150 as well as pyrrolysyl-tRNAcuA. Individual colonies (96) were collected from the agar plate used to inoculate a 96-deep well plate containing 0.5 mL per well non-inducing media (NIM) containing 50 μg/mL Kn and 25 μg/mL Tcn. This block was cultured for 24 hrs. at 37° C. and 300 RPM. The non-inducing media saturated cells were used to inoculate into two 96-well plates containing 0.5 mL per well auto-inducing media (AIM) in presence and absence of Tet4 amino acids at 37° C. and 300 RPM. The cell culture and GFP expression was monitored by OD600 and fluorescence measurement at 24 hrs. and 48 hrs. This analysis led to four unique synthetases D4, E1, D10, and F2 which were able to selectively suppress the TAG interrupted GFP in presence of Tet4-Ph. None of the remaining pBK-library members were selective for encoding Tet4-Me.

Efficiency and fidelity of selected synthetases. The efficiency and fidelity of the selected synthetases were measured by expressing GFP150 in 50 mL AIM with and without 0.5 mM Tet4-Ph. DH10b cells containing the selected pBK-RSs and the pALS-GFP150TAG plasmids, were used to inoculate 5 mL of NIM containing kanamycin (50 μg/mL) and tetracycline (25 μg/mL). Cells were grown for 16 hours at 37° C. shaking at 250 rpm. The saturated NIM cultures (500 μL) were used to inoculate in 50 mL AIM containing kanamycin (50 μg/mL) and tetracycline (25 μg/mL) and measured fluorescence using a Turner Biosystems Picofluor fluorimeter diluting 100 μL cell culture in 1.9 mL water. Two synthetases D4 and E1 for Tet4-Ph were identified to have good efficiency and fidelity. The selected two synthetases are differ at two mutation sites C313A/G and W382I/V respectively.

Permissivity screen for Tet4-Pyr with selected synthetases. To assess if the top four selected RSs for Tet4-Ph could incorporate Tet4-Pyr, expressions of GFP150TAG with Tet4-Pyr were evaluated. Using the above efficiency and fidelity method, 5 mL AIM separate cultures were inoculated with cells containing the pBK-RSs/pALS-GFP150. Tet4-Pyr (0.5 mM) was added to cultures from a 50 mM DMF ncAA stock solution. Cultures were grown for 30-36 hours at 37° C. and 250 rpm. Fluorescence of the culture was measured by diluting 100 μL cell culture in 1.9 mL water.

Expression and purification of GFP150-TAG-Tet4. Using expression conditions above for efficiency and fidelity measurements, GFP150-Tet4 was expressed in 50 mL AIM and all cells were harvested after 36 hrs by centrifugation at 5000 rcf for 10 min. Media was removed, and cell pellets were stored at −80° C. Cells were resuspended in wash buffer (NaCl 300 mM, NaH₂PO₄ 15.5 mM, Na₂HPO₄ 34.5 mM, imidazole 5 mM, pH 7.1). Cells were lysed using a Microfluidics M-110P microfluidizer (18,000 psi) and the lysate was collected in wash buffer. The lysate was clarified by centrifugation (21000 rcf, 30 mins.) and to the clarified supernatant TALON resin (300 μL bed volume) was added. Lysate was incubated with the resin for 1-2 hours gently rocking at 4° C. Resin and lysate were applied to a column and flow through was discarded. Resin was washed 5 times with 10 mL wash buffer. Protein was eluted with 250 μL elution buffer (NaCl 300 mM, NaH₂PO₄ 15.5 mM, Na₂HPO₄ 34.5 mM, imidazole 250 mM, pH 7.0). Protein concentration was determined by measuring absorbance at 280 nm. Protein purity was assessed using SDS-PAGE.

Mobility Shift Assay. The purified GFP-Wt, GFP150-Tet4-Ph/Pyr, and MBP-Tet4-Ph variants were diluted to 50 μM in PBS. The protein was reacted with excess sTCO-PEG5k (500 μM) for 5-10 minutes in PBS at room temperature. Protein was denatured through the addition of Laemmli buffer and heated at 95° C. for 10 minutes. Samples were then analyzed using a 12% SDS-PAGE gel.

Mass spectra of GFP-Tet4. Purified GFP-TAG150-Tet4 was diluted to 50 μM and desalted using Zeba™ spin desalting column and analyzed using an FT LTQ mass spectrometer at the Oregon State University mass spectrometry facility. Waters SYNAPT G2 HDMS with a Waters Acquity I class UPLC mass spectrometer was used to verify reaction of purified GFP-TAG150-Tet4 with sTCO. Samples were run 45 minutes gradient with H₂O:ACN:0.1% formic acid using a Thermo Scientific-MAbPac™RP column (2.1×100 mm and a 0.2 ml/min flow rate). Spectra were deconvoluted using the Maximum Entropy deconvolution algorithm (MaxEnt3) in Waters MassLynx software.

Measuring reaction rates of GFP-Tet4-Ph/Pyr with sTCO-OH and sTCO-spin labels. Fluorescence dequenching of pure GFP-Tet protein by reaction with sTCO-reagents was used to measure the rates of Tet reaction on proteins. The fluorescence of GFP-Tet4-Ph and GFP-Tet4-Pyr in 3 mL of PBS (12 nmol) was measured (488 nm excitation, 509 nm emission, 5 points/second) for 50 seconds prior to the addition of 10 μL sTCO and sTCO-spin labels (0.03-3 μmol sTCO reagent in PBS). Stock concentrations (10-900 μmol) of sTCO reagents were prepared in methanol. Reactions were monitored until the return of fluorescence stabilized. Curves were fitted to a single exponential equation using the curve-fitting program OriginPro 8.5 to determine kinetic constants.

### Maltose Binding Protein (MBP)-Tet4-pH Expression and Purification for Spin Labeling

MBP cloning. The wild type (Wt) and TAG variants (211, 278, 295, 322, 211-295 and 278-322) of the MBP genes with N-terminus His-tag were cloned in place of the GFP gene in the pALS plasmid. The MBP genes were amplified form the pE™-11 vectors⁸ using the primers pALS-MBP-For and pALS-MBP-Rev and incorporated into the pALS plasmid at the cut sites XhoI and XbaI.

pALS-MBP-For
(SEQ ID NO: 9)
(5′GTTTTTTGGGCTAACAGGAGGAATTAACATG
AAACATCACCATCACCATCACCCCA-3′)
pALS-MBP-Rev
(SEQ ID NO. 10)
(5′GAGTTTTTGTTCGGGCNCAAGCTTCGCTCGA
GTTATTATTTGGTGATGCGAGTCTGC-3′)

E. coli MBP-Tet4-Ph expression and purification. Single colonies of DH10B cells co-transformed with pBK-E1 and single/double TAG mutant pALS-MBP plasmids (pALS-MBP-Wt; pALS-MBP-211; pALS-MBP-278; pALS-MBP-295; pALS-MBP-322; pALS-MBP-211/295; pALS-MBP-278/322) were used to inoculate a 5 mL non-inducing culture containing 50 μg/mL Kn and 25 μg/mL Tet. The non-inducing culture was grown to saturation, shaking at 250 rpm and 37° C. Autoinduction media (50 mL) with 50 μg/mL Kn and 25 μg/mL Tet, was inoculated with 0.7 mL of saturated non-inducing culture. When autoinducing cultures reached and OD of 0.5-0.7 0.3 mM Tet4-Ph was added. After 30 hours of shaking at 37° C., cells were collected by centrifugation and stored at −80° C. The cell pellets were resuspended in TALON wash buffer (pH˜7.4) with 5 mM imidazole and lysed using a microfluidizer (final volume 30 mL). Cell lysate was clarified by centrifugation and added to TALON cobalt resin (0.2 mL bed-volume) and incubated for 1 hr at 4° C. Bound resin was washed with >50 mL volumes wash buffer. Protein was eluted by using 0.3 mL elution buffer containing 250 mM imidazole (pH 7.1). Purified protein yields per liter of media were MBP-Wt (120 mg/L), MBP-211 (15 mg/L), MBP-295 (25 mg/L), MBP-278 (32 mg/L), MBP-322 (45 mg/L), MBP-211/295 (10 mg/L), MBP-278/322 (14 mg/L).

HEK293T MBP211/295(W340A)-Tet4-Ph expression and purification. HEK293T cells in a 500 cm² dish were transfected with 1.5 μg/mL DNA (5:1:1.2 ratio of pAcBac1 FLAG-MBP(W340A)211/295-TAG:pAcBac1 MbRS E1:pcDNA1 eRF1 E55D) and PEI “Max” 40K (Polysciences) in a 3:1 PEI:DNA ratio. Tet4-Ph ncAA was supplemented in the growth medium at 100 μM. 32 hours post-transfection, cells were washed once with PBS, detached from the surface with 3 mL 0.05% trypsin, 1 mM EDTA (2 min), and harvested in 1:1 PBS:DMEM+10% FBS. Cells were pelleted by centrifugation at 300×g, washed once with PBS, and pelleted at 300×g. Cell pellet was stored overnight at −20° C.

Cells were lysed by sonication in buffer containing 50 mM Tris pH 7.4, 150 mM NaCl, and 1 mM EDTA with protease inhibitor cocktail (Pierce). Cleared lysate was incubated with a 2 mL bed volume of FLAG M2 affinity resin (Sigma) and nutated at 4° C. for 6 h. Resin was washed with 20 column volumes of wash buffer (50 mM Tris pH 7.4, 150 mM NaCl, 0.5 mM EDTA) and protein was eluted in wash buffer plus 200 μg/mL FLAG peptide. Eluted protein was desalted (PD-10) into 25 mM Tris pH 7.4, 10 mM NaCl and further purified by anion exchange on a Q-Sepharose HP column with an elution gradient to 1M NaCl over 20 column volumes. Eluted protein was desalted into 25 mM HEPES pH 7.2, 150 mM NaCl, concentrated (30 kDa MWCO), and spin-labeled as described below.

In vitro spin-labeling. Purified single and double cysteine constructs of MBP were incubated at room temperature with 10 mM tris(2-carboxyethyl)phosphine for 10 minutes, concentrated with a 50-kDa molecular-weight cutoff (MWCO) spin concentrator (GE Healthcare) and purified by size exclusion chromatography (SEC) on a Superdex 200 10/300 GL column (GE Healthcare) equilibrated with EPR sample buffer (150 mM NaCl, 25 mM HEPES, pH 7.2). SEC fractions were immediately reacted with MTSL (Toronto Research Chemicals) at room temperature for about 4 h, protected from light. Unreacted spin label was removed by desalting (PD-10) into EPR sample buffer and concentrated (50-kDa MWCO). Removal of residual free spin label and exchange into deuterated buffer (for DEER samples) was achieved by microdialysis (Pierce). Glycerol (CW samples) or d8-glycerol (DEER samples) was supplemented to a final concentration of 30% (vol/vol). For samples containing maltose, D-(+)-maltose was included at 1-5 mM, depending on the experiment. Samples for CW EPR were loaded into 1.0 mm outer diameter (OD), 0.7 mm inner diameter (ID) quartz capillaries (Sutter), sealed with wax, and maintained at room temperature until measurement. Samples for DEER spectroscopy were loaded into 1.5 mm OD/1.1 mm ID quartz tubes (Sutter) with flame-sealed bottoms and flash frozen in liquid nitrogen. Samples were stored at −80° C. until measurement.

For labeling of single or double GFP and MBP constructs containing Tet4-Ph, purified proteins were desalted into EPR sample buffer (PD-10) and reacted with 100-250 μM sTCO-tM6, sTCO-tM5, or sTCO-tE5 for 1 h at room temperature. Removal of free spin label and EPR sample preparation was performed in the same manner as described above for MTSL-labeled constructs.

In-cell spin-labeling. Free Tet4 ncAA was removed from HEK293T cells expressing Tet4-Ph constructs of GFP or MBP by exchanging the culture medium 3 times with fresh DMEM+10% FBS over the course of 4-6 h with incubation at 37° C., 5% CO₂. Following the final incubation, medium was removed and replaced with medium supplemented with 100-250 nM sTCO-tE5, depending on the experiment. Cells were incubated in labeling medium at room temperature for 10-15 minutes, after which the labeling medium was removed and the cells were washed once with PBS, detached from the surface with PBS+5 mM EDTA and collected by centrifugation at 300×g. Cell pellets were gently resuspended in a minimum volume of PBS+10% glycerol (GFP sample) or DMEM+10% FBS+10% glycerol (MBP sample). For CW EPR measurements, cells were loaded into 1.0 mm OD, 0.7 mm ID quartz capillaries, sealed with wax, briefly pelleted by centrifugation at 300×g and immediately taken to the spectrometer for recording. For DEER experiments, resuspended cells were transferred to a 3 mm OD quartz EPR tube, pelleted by centrifugation at 100×g and frozen in liquid N₂. DEER samples were stored at −80° C. until measurement.

EPR spectroscopy. Continuous-wave EPR spectra were recorded at room temperature on a Bruker EMX spectrometer operating at X-band frequency (about 9.8 GHz) equipped with a Bruker ER 4123D dielectric resonator. Spectra were recorded with 100 kHz field modulation with a sweep rate of 1.8 G/s and a modulation amplitude of 2 G. CW EPR spectra were background-subtracted and baseline corrected in LabVIEW™. Spin concentrations were calculated by double integration of the field-modulated spectrum and comparison to a standard curve of TEMPO free radical (Sigma). Labeling efficiencies were calculated as the spin concentration obtained by double integration divided by the total protein concentration estimated from optical absorbance at 280 nm using an extinction coefficient for 6×His-MBP of 67,280 M¹cm⁻¹. Reported labeling efficiencies are the average efficiencies between apo and maltose-bound samples for each construct.

Pulsed EPR experiments were performed at Q-band frequency (about 34 GHz) using a Bruker EleXsys E580 spectrometer with an overcoupled Bruker EN 5107D2 (in vitro samples) or ER 5106-QT2 (in-cell samples) resonator. Pulses were generated with a Bruker SpinJet AWG and amplified with a 300 W TWT amplifier (Applied Systems Engineering). DEER experiments were performed at 45-50 K using a variable-temperature cryogen-free system (Bruker/ColdEdge). The deadtime-free, four-pulse DEER sequence [(π/2)_{probe}−τ₁−(π)_{probe}−τ₁+t−(π)ₚᵤₘₚ−τ₂−t−(π)_{probe}−τ₂−(echo)] was employed with a 260 ns or 400 ns τ₁ delay and τ₂ delays ranging from 2-6 μs depending on the sample. For samples in deuterated buffer, τ₁ delays were incremented by 16 ns over 8 steps to suppress deuterium ESEEM contributions to the DEER trace. The pump pulse was implemented as a 150 ns sech/tanh pulse with a frequency bandwidth of 80 MHz and a truncation parameter (β) of 10. The magnetic field was adjusted such that the pump pulse was centered near the maximum of the nitroxide field-swept spectrum. Probe pulses were 60 ns (π/2 and π) Gaussian-shaped pulses applied at a frequency 80 MHz below that of the pump pulse center frequency. All pulses were compensated for resonator bandwidth using the pulse function of EasySpin-v6.0-dev. Acquisition times for in-cell DEER experiments were 224 h (GFP) and 264 h (MBP). DEER acquisition times for purified proteins were typically 12-48 h depending on the sample. Raw time-domain DEER traces were background-corrected and distance distributions were calculated by Tikhonov regularization in LongDistances (by Christian Altenbach, available at www.biochemistry.ucla.edu/Faculty/Hubbell). Error bands in the distance distribution were calculated by stochastic addition of random noise to the DEER signal and variation of the background parameters followed by recalculation of the distance distribution using the error analysis feature of LongDistances with default values. Error bands are plotted as the mean±1 standard deviation from 100-200 independent calculations with parameters varied as described above.

In silico spin label modeling. Spin-labeled side chain ensembles of Tet4-Ph-tE5 were modeled onto MBP and GFP structures using chiLife, an open-source Python package for site-directed spin label modeling. Tet4-Ph-tE5 ncAA with N-terminal acetylation and C-terminal amidation was constructed as the 1,4-dihydropyridazine product with cis fusion of the cyclopropane ring and the eight-membered ring set to the ‘half-chair’ conformation. A rotamer library consisting of 601 low-energy structures was generated using the GFN-FF force field in CREST. Rotamer ensembles and simulated DEER distance distributions for each site-pair were generated in chiLife using off-rotamer sampling of mobile dihedral angles using the default values.

### Eukaryotic Expression Materials and Methods

Toxicity Screen of Tetrazines. HEK293T cells were plated in a 96-well plate at approximately 10% confluency. Cells were incubated for 48 h with Tet4 ncAA or 1% DMSO, and the cell viability was measured using CellTiter Glo assay kit (Promega) according to the manufacturer's instruction. Briefly, 25 μL of CellTiter Glo reagent was added to each well and incubated for 10 min at RT. The signal was measured for 1 sec using TR717 microplate luminometer (Berthold, Germany) and WinGlow software version 1.25 (Berthold Technologies). The data was normalized to vehicle control and fit to a curve using non-linear regression method with GraphPad Prism 5. n=3±95% CI.

Transfection of HEK293T cells and imaging method. HEK293T cells were plated in a 24-well plate at the density of 40% confluency. Cells were transfected the next day using jetPRIME reagent according to the manufacturer's protocol. Briefly, 67 ng of pAcBac1-NES-D4-RS or pAcBac1-NES-E1-RS and 555 ng of pAcBac1-GFP-150TAG plasmid DNA were diluted with 50 μL of jetPRIME buffer. To the diluted DNA, 1.2 uL of jetPRIME reagent was added and vortexed. The complex was incubated for 10 min at room temperature. It was gently added to the cells. Tet4 ncAA or 0.1% DMSO were added to the cells immediately. Cells were incubated 18 h-48 h before analysis. GFP expression was verified by fluorescence microscopy on EVOS FL imaging system.

Live-cell spin-labeling—fluorescence microscopy. HEK293T/17 cells expressing GFP150-Tet4-Ph were plated on circular (25 mm diameter) poly-(D)-lysine-coated microscope glass coverslips and incubated in DMEM+10% FBS until the desired cell density was reached. Coverslips were then encased within a homebuilt perfusion device and bathed in DMEM+10% FBS. Live cells were imaged using a Nikon Eclipse TE2000-E microscope with a 10× water immersion objective. GFP was excited using epifluorescence with a Lambda SC SmartShutter controller (Sutter Instruments) with 470/40 nm excitation and 515/30 nm emission filters. GFP fluorescence was recorded with 10 ms exposures using an Evolve 512 EMCCD camera (Photometrics) and MetaMorph software (Molecular Devices). Cells were manually perfused with DMEM+10% FBS medium containing 1 μM sTCO-tE5 spin label, and cells were imaged every 500 ms for a total of 240 images. Mean cell fluorescence for each timepoint was calculated by first defining a region of interest (ROI) for each cell based on the final image in the time series using the particle analysis tool in ImageJ. Particle ROIs were then applied across all images in the time series. Particle intensities for each ROI were background-subtracted and the mean particle intensity was calculated for each image. Mean fluorescence values were then normalized by the mean fluorescence value from the first image, recorded prior to application of spin label.

### Sequence Listings

GFP-Wt (Protein)
(SEQ ID NO: 1)
MVSKGEELFTGVVPILVELDGDVNGHKFSVRGEGEGDATNGKLTL
KFICTTGKLPVPWPTLVTTLTYGVQCFSRYPDHMKRHDFFKSAMP
EGYVQERTISFKDDGTYKTRAEVKFEGDTLVNRIELKGIDFKEDG
NILGHKLEYNFNSHNVYITADKQKNGIKANFKIRHNVEDGSVQLA
DHYQQNTPIGDGPVLLPDNHYLSTQSVLSKDPNEKRDHMVLLEFV
TAAGITHGMDELYKGSHHHHHH
GFP-Wt (DNA)
(SEQ ID NO: 2)
ATGGTTAGCAAAGGTGAAGAACTGTTTACCGGCGTTGTGCCGATT
CTGGTGGAACTGGATGGTGATGTGAATGGCCATAAATTTAGCGTT
CGTGGCGAAGGCGAAGGTGATGCGACCAACGGTAAACTGACCCTG
AAATTTATTTGCACCACCGGTAAACTGCCGGTTCCGTGGCCGACC
CTGGTGACCACCCTGACCTATGGCGTTCAGTGCTTTAGCCGCTAT
CCGGATCATATGAAACGCCATGATTTCTTTAAAAGCGCGATGCCG
GAAGGCTATGTGCAGGAACGTACCATTAGCTTCAAAGATGATGGC
ACCTATAAAACCCGTGCGGAAGTTAAATTTGAAGGCGATACCCTG
GTGAACCGCATTGAACTGAAAGGTATTGATTTTAAAGAAGATGGC
AACATTCTGGGTCATAAACTGGAATATAATTTCAACAGCCATAAT
GTGTATATTACCGCCGATAAACAGAAAAATGGCATCAAAGCGAAC
TTTAAAATCCGTCACAACGTGGAAGATGGTAGCGTGCAGCTGGCG
GATCATTATCAGCAGAATACCCCGATTGGTGATGGCCCGGTGCTG
CTGCCGGATAATCATTATCTGAGCACCCAGAGCGTTCTGAGCAAA
GATCCGAATGAAAAACGTGATCATATGGTGCTGCTGGAATTTGTT
ACCGCCGCGGGCATTACCCACGGTATGGATGAACTGTATAAAGGC
AGCCACCATCATCATCACCATTAA
E1-Py1RS (Protein)
(SEQ ID NO: 3)
MDKKPLDVLISATGLWMSRTGTLHKIKHHEVSRSKIYIEMACGDH
LVVNNSRSCRTARAFRHHKYRKTCKRCRVSDEDINNFLTRSTESK
NSVKVRVVSAPKVKKAMPKSVSRAPKPLENSVSAKASTNTSRSVP
SPAKSTPNSSVPASAPAPSLTRSQLDRVEALLSPEDKISLNMAKP
FRELEPELVTRRKNDFQRLYTNDREDYLGKLERDITKFFVDRGFL
EIKSPILIPAEYVERMGINNDTELSKQIFRVDKNLCLRPMLAPTL
YNYLRKLDRILPGPIKVFEVGPCYRKESDGKEHLEEFTMVSFGQM
GSGCTRENLEALIKEFLDYLEIDFEIVGDSCMVYGDTLDIMHGDL
ELSSAHVGPVSLDREWGIDKPVIGAGFGLERLLKVMHGFKNIKRA
SRSESYYNGISTNL
E1-Py1RS (DNA)
(SEQ ID NO: 4)
ATGGATAAAAAACCGCTGGATGTGCTGATTAGCGCGACCGGCCTG
TGGATGAGCCGTACCGGCACCCTGCATAAAATCAAACATCATGAA
GTGAGCCGCAGCAAAATCTATATTGAAATGGCGTGCGGCGATCAT
CTGGTGGTGAACAACAGCCGTAGCTGCCGTACCGCGCGTGCGTTT
CGTCATCATAAATACCGCAAAACCTGCAAACGTTGCCGTGTGAGC
GATGAAGATATCAACAACTTTCTGACCCGTAGCACCGAAAGCAAA
AACAGCGTGAAAGTGCGTGTGGTGAGCGCGCCGAAAGTGAAAAAA
GCGATGCCGAAAAGCGTGAGCCGTGCGCCGAAACCGCTGGAAAAT
AGCGTGAGCGCGAAAGCGAGCACCAACACCAGCCGTAGCGTTCCG
AGCCCGGCGAAAAGCACCCCGAACAGCAGCGTTCCGGCGTCTGCG
CCGGCACCGAGCCTGACCCGCAGCCAGCTGGATCGTGTGGAAGCG
CTGCTGTCTCCGGAAGATAAAATTAGCCTGAACATGGCGAAACCG
TTTCGTGAACTGGAACCGGAACTGGTGACCCGTCGTAAAAACGAT
TTTCAGCGCCTGTATACCAACGATCGTGAAGATTATCTGGGCAAA
CTGGAACGTGATATCACCAAATTTTTTGTGGATCGCGGCTTTCTG
GAAATTAAAAGCCCGATTCTGATTCCGGCGGAATATGTGGAACGT
ATGGGCATTAACAACGACACCGAACTGAGCAAACAAATTTTCCGC
GTGGATAAAAACCTGTGCCTGCGTCCGATGCTGGCCCCGACCCTG
TATAACTATCTGCGTAAACTGGATCGTATTCTGCCGGGTCCGATC
AAAGTTTTTGAAGTGGGCCCGTGCTATCGCAAAGAAAGCGATGGC
AAAGAACACCTGGAAGAATTCACCATGGTTTCGTTTGGTCAAATG
GGCAGCGGCTGCACCCGTGAAAACCTGGAAGCGCTGATCAAAGAA
TTCCTGGATTATCTGGAAATCGACTTCGAAATTGTGGGCGATAGC
TGCATGGTGTATGGCGATACCCTGGATATTATGCATGGCGATCTG
GAACTGAGCAGCGCGCATGTGGGTCCGGTTAGCCTGGATCGTGAA
TGGGGCATTGATAAACCGGTGATTGGCGCGGGTTTTGGCCTGGAA
CGTCTGCTGAAAGTGATGCATGGCTTCAAAAACATTAAACGTGCG
AGCCGTAGCGAAAGCTACTATAACGGCATTAGCACGAACCTGTAA
D4-Py1RS (Protein)
(SEQ ID NO: 5)
MDKKPLDVLISATGLWMSRTGTLHKIKHHEVSRSKIYIEMACGDH
LVVNNSRSCRTARAFRHHKYRKTCKRCRVSDEDINNFLTRSTESK
NSVKVRVVSAPKVKKAMPKSVSRAPKPLENSVSAKASTNTSRSVP
SPAKSTPNSSVPASAPAPSLTRSQLDRVEALLSPEDKISLNMAKP
FRELEPELVTRRKNDFQRLYTNDREDYLGKLERDITKFFVDRGFL
EIKSPILIPAEYVERMGINNDTELSKQIFRVDKNLCLRPMLAPTL
YNYLRKLDRILPGPIKIFEVGPCYRKESDGKEHLEEFTMVSFAQM
GSGCTRENLEALIKEFLDYLEIDFEIVGDSCMVYGDTLDIMHGDL
ELSSAHVGPVSLDREWGIDKPIIGAGFGLERLLKVMHGFKNIKRA
SRSESYYNGISTNL
D4-Py1RS (DNA)
(SEQ ID NO: 6)
ATGGATAAAAAACCGCTGGATGTGCTGATTAGCGCGACCGGCCTG
TGGATGAGCCGTACCGGCACCCTGCATAAAATCAAACATCATGAA
GTGAGCCGCAGCAAAATCTATATTGAAATGGCGTGCGGCGATCAT
CTGGTGGTGAACAACAGCCGTAGCTGCCGTACCGCGCGTGCGTTT
CGTCATCATAAATACCGCAAAACCTGCAAACGTTGCCGTGTGAGC
GATGAAGATATCAACAACTTTCTGACCCGTAGCACCGAAAGCAAA
AACAGCGTGAAAGTGCGTGTGGTGAGCGCGCCGAAAGTGAAAAAA
GCGATGCCGAAAAGCGTGAGCCGTGCGCCGAAACCGCTGGAAAAT
AGCGTGAGCGCGAAAGCGAGCACCAACACCAGCCGTAGCGTTCCG
AGCCCGGCGAAAAGCACCCCGAACAGCAGCGTTCCGGCGTCTGCG
CCGGCACCGAGCCTGACCCGCAGCCAGCTGGATCGTGTGGAAGCG
CTGCTGTCTCCGGAAGATAAAATTAGCCTGAACATGGCGAAACCG
TTTCGTGAACTGGAACCGGAACTGGTGACCCGTCGTAAAAACGAT
TTTCAGCGCCTGTATACCAACGATCGTGAAGATTATCTGGGCAAA
CTGGAACGTGATATCACCAAATTTTTTGTGGATCGCGGCTTTCTG
GAAATTAAAAGCCCGATTCTGATTCCGGCGGAATATGTGGAACGT
ATGGGCATTAACAACGACACCGAACTGAGCAAACAAATTTTCCGC
GTGGATAAAAACCTGTGCCTGCGTCCGATGCTGGCCCCGACCCTG
TATAACTATCTGCGTAAACTGGATCGTATTCTGCCGGGTCCGATC
AAAATTTTTGAAGTGGGCCCGTGCTATCGCAAAGAAAGCGATGGC
AAAGAACACCTGGAAGAATTCACCATGGTTTCGTTTGCTCAAATG
GGCAGCGGCTGCACCCGTGAAAACCTGGAAGCGCTGATCAAAGAA
TTCCTGGATTATCTGGAAATCGACTTCGAAATTGTGGGCGATAGC
TGCATGGTGTATGGCGATACCCTGGATATTATGCATGGCGATCTG
GAACTGAGCAGCGCGCATGTGGGTCCGGTTAGCCTGGATCGTGAA
TGGGGCATTGATAAACCGATTATTGGCGCGGGTTTTGGCCTGGAA
CGTCTGCTGAAAGTGATGCATGGCTTCAAAAACATTAAACGTGCG
AGCCGTAGCGAAAGCTACTATAACGGCATTAGCACGAACCTGTAA
Py1-tRNA (DNA sequence)
(SEQ ID NO: 11)
TGGCGGAAACCCCGGGAATCTAACCCGGCTGAACGGATTTAGAGT
CCATTCGATCTACATGATCAGGTTCCC
MBP-Wt (Protein)
(SEQ ID NO: 7)
MKHHHHHHPMSDYDIPTTENLYFQGAMAKTEEGKLVIWINGDKGY
NGLAEVGKKFEKDTGIKVTVEHPDKLEEKFPQVAATGDGPDIIFW
AHDRFGGYAQSGLLAEITPDKAFQDKLYPFTWDAVRYNGKLIAYP
IAVEALSLIYNKDLLPNPPKTWEEIPALDKELKAKGKSALMENLQ
EPYFTWPLIAADGGYAFKYENGKYDIKDVGVDNAGAKAGLTFLVD
LIKNKHMNADTDYSIAEAAFNKGETAMTINGPWAWSNIDTSKVNY
GVTVLPTFKGQPSKPFVGVLSAGINAASPNKELAKEFLENYLLTD
EGLEAVNKDKPLGAVALKSYEEELAKDPRIAATMENAQKGEIMPN
IPQMSAFWYAVRTAVINAASGRQTVDEALKDAQTRITK
MBP-Wt (DNA)
(SEQ ID NO: 8)
ATGAAACATCACCATCACCATCACCCCATGAGCGATTACGACATC
CCCACTACTGAGAATCTTTATTTTCAGGGCGCCATGGCGAAAACT
GAAGAAGGTAAACTGGTAATCTGGATTAACGGCGATAAAGGCTAT
AACGGTCTCGCTGAAGTCGGTAAGAAATTCGAGAAAGATACAGGA
ATTAAAGTCACCGTTGAGCATCCGGATAAACTGGAAGAGAAATTC
CCACAGGTTGCGGCAACTGGCGATGGCCCTGACATTATCTTCTGG
GCACACGACCGCTTTGGTGGCTACGCTCAATCTGGCCTGTTGGCT
GAAATCACCCCGGACAAAGCGTTCCAGGACAAGCTGTATCCGTTC
ACCTGGGATGCCGTACGTTACAACGGCAAGCTGATTGCTTACCCG
ATCGCTGTTGAAGCGTTATCGCTGATTTATAACAAAGATCTGCTG
CCGAACCCGCCAAAAACCTGGGAAGAGATCCCGGCGCTGGATAAA
GAACTGAAAGCGAAAGGTAAGAGCGCGCTGATGTTCAACCTGCAA
GAACCGTACTTCACCTGGCCGCTGATTGCTGCTGACGGGGGTTAT
GCGTTCAAGTATGAAAACGGCAAGTACGACATTAAAGACGTGGGC
GTGGATAACGCTGGCGCGAAAGCGGGTCTGACCTTCCTGGTTGAC
CTGATTAAAAACAAACACATGAATGCAGACACCGATTACTCCATC
GCAGAAGCTGCCTTTAATAAAGGCGAAACAGCGATGACCATCAAC
GGCCCGTGGGCATGGTCCAACATCGACACCAGCAAAGTGAATTAT
GGTGTAACGGTACTGCCGACCTTCAAGGGTCAACCATCCAAACCG
TTCGTTGGCGTGCTGAGCGCAGGTATTAACGCCGCCAGTCCGAAC
AAAGAGCTGGCAAAAGAGTTCCTCGAAAACTATCTGCTGACTGAT
GAAGGTCTGGAAGCGGTTAATAAAGACAAACCGCTGGGTGCCGTA
GCGCTGAAGTCTTACGAGGAAGAGTTGGCGAAAGATCCACGTATT
GCCGCCACTATGGAAAACGCCCAGAAAGGTGAAATCATGCCGAAC
ATCCCGCAGATGTCCGCTTTCTGGTATGCCGTGCGTACTGCGGTG
ATCAACGCCGCCAGCGGTCGTCAGACTGTCGATGAAGCCCTGAAA
GACGCGCAGACTCGCATCACCAAATAA
Mb-Pyl-tRNA (DNA sequence)
(SEQ ID NO: 11)
TGGCGGAAACCCCGGGAATCTAACCCGGCTGAACGGATTTAGAGT
CCATTCGATCTACATGATCAGGTTCCC
Ma tRNA sequence (top strand) for Tet4.0
p-Aminophenyl encoding with
MaA4RS
(SEQ ID NO: 12)
5′_AGATCTGGGGGACGGTCCGGCGACCAGCGGGTCTCTAAAACC
TAGCATAGCGGGGTTCGACACCCCGGTCTCTCG_3′
DNA sequence (top strand) for MaA4RS
selected for Tet4.0_p-Aminophenyl
encoding
(SEQ ID NO: 13)
5′_ATGACAGTGAAATACACAGATGCCCAGATCCAGCGCCTGCGG
GAGTATGGCAACGGCACCTATGAGCAGAAGGTGTTTGAAGATCTG
GCCTCTAGAGATGCAGCCTTCTCCAAGGAGATGTCCGTGGCTTCC
ACAGACAACGAGAAAAAGATCAAGGGCATGATTGCCAACCCCAGC
CGCCATGGGCTGACCCAGCTGATGAATGACATCGCCGACGCCCTG
GTGGCCGAGGGCTTCATCGAGGTCAGGACCCCCATCTTCATTTCT
AAGGACGCGCTGGCTCGCATGACCATCACCGAGGACAAGCCCCTG
TTCAAGCAGGTGTTCTGGATCGATGAGAAGAGGGCTCTGAGGCCC
ATGCTCGCCCCCAACCAGTACTCCGTGATGCGGGACCTGcgcGAC
CACACGGACGGCCCTGTGAAAATTTTCGAAATGGGCTCCTGCTTT
AGGAAAGAAAGCCACAGCGGAATGCACCTGGAGGAGTTCACCATG
CTGAGTCTGTGTGACATGGGGCCAAGAGGAGATGCCACAGAAGTG
CTGAAGAACTACATCTCAGTGGTCATGAAGGCTGCTGGACTGCCC
GACTATGATTTGGTGCAGGAAGAGAGCGATGTCTACAAAGAaACC
ATTGATGTGGAGATCAATGGCCAGGAGGTGTGCTCTGCTTGTGTG
GGCCCCCACTACCTGGACGCCGCCCACGACGTGCATGAACCCTAT
AGTGGAGCGGGCTTTGGCCTGGAGAGGCTGCTGACCATAAGAGAA
AAGTACAGCACTGTGAAGAAAGGCGGCGCCTCCATCTCCTACTTG
AATGGAGCCAAGATCAACAGCGGCTGA_3′
Amino acid sequence of MaA4RS selected
for Tet4.0 p-Aminophenyl encoding
(SEQ ID NO: 14)
MTVKYTDAQIQRLREYGNGTYEQKVFEDLASRDAAFSKEMSVAST
DNEKKIKGMIANPSRHGLTQLMNDIADALVAEGFIEVRTPIFISK
DALARMTITEDKPLFKQVFWIDEKRALRPMLAPNQYSVMRDLRDH
TDGPVKIFEMGSCFRKESHSGMHLEEFTMLSLCDMGPRGDATEVL
KNYISVVMKAAGLPDYDLVQEESDVYKETIDVEINGQEVCSACVG
PHYLDAAHDVHEPYSGAGFGLERLLTIREKYSTVKKGGASISYLN
GAKINSG*
Ma tRNA sequence (top strand) for Tet4.0
Dihydropyran (DHP) encoding with
MaD1RS
(SEQ ID NO: 15)
5′_AGATCTGGGGGACGGTCCGGCGACCAGCGGGTCTCTAAAACC
TAGCATAGCGGGGTTCGACACCCCGGTCTCTCG_3′
DNA sequence (top strand) for MaD1RS
selected for Tet4.0-Dihydropyran (DHP)
encoding
(SEQ ID NO: 16)
5′_ATGACAGTGAAATACACAGATGCCCAGATCCAGCGCCTGCGG
GAGTATGGCAACGGCACCTATGAGCAGAAGGTGTTTGAAGATCTG
GCCTCTAGAGATGCAGCCTTCTCCAAGGAGATGTCCGTGGCTTCC
ACAGACAACGAGAAAAAGATCAAGGGCATGATTGCCAACCCCAGC
CGCCATGGGCTGACCCAGCTGATGAATGACATCGCCGACGCCCTG
GTGGCCGAGGGCTTCATCGAGGTCAGGACCCCCATCTTCATTTCT
AAGGACGCGCTGGCTCGCATGACCATCACCGAGGACAAGCCCCTG
TTCAAGCAGGTGTTCTGGATCGATGAGAAGAGGGCTCTGAGGCCC
ATGCTCGCCCCCAACCAGTACTCCGTGATGCGGGACCTGcgcGAC
CACACGGACGGCCCTGTGAAAATTTTCGAAATGGGCTCCTGCTTT
AGGAAAGAAAGCCACAGCGGAATGCACCTGGAGGAGTTCACCATG
CTGTCTCTGGTTGACATGGGGCCAAGAGGAGATGCCACAGAAGTG
CTGAAGAACTACATCTCAGTGGTCATGAAGGCTGCTGGACTGCCC
GACTATGATTTGGTGCAGGAAGAGAGCGATGTCTACAAAGAaACC
ATTGATGTGGAGATCAATGGCCAGGAGGTGTGCTCTGCTAGTGTG
GGCCCCCACTACCTGGACGCCGCCCACGACGTGCATGAACCCTGG
AGTGGAGCGGGCTTTGGCCTGGAGAGGCTGCTGACCATAAGAGAA
AAGTACAGCACTGTGAAGAAAGGCGGCGCCTCCATCTCCTACTTG
AATGGAGCCAAGATCAACAGCGGCTGA_3′
Amino acid sequence of MaD1RS selected
for Tet4.0-Dihydropyran (DHP) encoding
(SEQ ID NO: 17)
MTVKYTDAQIQRLREYGNGTYEQKVFEDLASRDAAFSKEMSVAST
DNEKKIKGMIANPSRHGLTQLMNDIADALVAEGFIEVRTPIFISK
DALARMTITEDKPLFKQVFWIDEKRALRPMLAPNQYSVMRDLRDH
TDGPVKIFEMGSCFRKESHSGMHLEEFTMLSLVDMGPRGDATEVL
KNYISVVMKAAGLPDYDLVQEESDVYKETIDVEINGQEVCSASVG
PHYLDAAHDVHEPWSGAGFGLERLLTIREKYSTVKKGGASISYLN
GAKINSG*
Ma tRNA sequence (top strand) for Tet4.0-
Phenyl and Tet4.0 p-nitrophenyl
encoding with MaE2RS
(SEQ ID NO: 18)
5′_AGATCTGGGGGACGGTCCGGCGACCAGCGGGTCTCTAAAACC
TAGCATAGCGGGGTTCGACACCCCGGTCTCTCG_3′
DNA sequence (top strand) for MaE2RS selected
for Tet4.0-Phenyl and Tet4.0 p-
nitrophenyl encoding
(SEQ ID NO: 19)
5′_ATGACAGTGAAATACACAGATGCCCAGATCCAGCGCCTGCGG
GAGTATGGCAACGGCACCTATGAGCAGAAGGTGTTTGAAGATCTG
GCCTCTAGAGATGCAGCCTTCTCCAAGGAGATGTCCGTGGCTTCC
ACAGACAACGAGAAAAAGATCAAGGGCATGATTGCCAACCCCAGC
CGCCATGGGCTGACCCAGCTGATGAATGACATCGCCGACGCCCTG
GTGGCCGAGGGCTTCATCGAGGTCAGGACCCCCATCTTCATTTCT
AAGGACGCGCTGGCTCGCATGACCATCACCGAGGACAAGCCCCTG
TTCAAGCAGGTGTTCTGGATCGATGAGAAGAGGGCTCTGAGGCCC
ATGCTCGCCCCCAACCAGTACTCCGTGATGCGGGACCTGcgcGAC
CACACGGACGGCCCTGTGAAAATTTTCGAAATGGGCTCCTGCTTT
AGGAAAGAAAGCCACAGCGGAATGCACCTGGAGGAGTTCACCATG
CTGAGTCTGGTGGACATGGGGCCAAGAGGAGATGCCACAGAAGTG
CTGAAGAACTACATCTCAGTGGTCATGAAGGCTGCTGGACTGCCC
GACTATGATTTGGTGCAGGAAGAGAGCGATGTCTACAAAGAaACC
ATTGATGTGGAGATCAATGGCCAGGAGGTGTGCTCTGCTGCCGTG
GGCCCCCACTACCTGGACGCCGCCCACGACGTGCATGAACCCTGG
AGTGGAGCGGGCTTTGGCCTGGAGAGGCTGCTGACCATAAGAGAA
AAGTACAGCACTGTGAAGAAAGGCGGCGCCTCCATCTCCTACTTG
AATGGAGCCAAGATCAACAGCGGCTGA_3′
Amino acid sequence of MaE2RS selected for
Tet4.0-Phenyl and Tet4.0_p-nitrophenyl encoding
(SEQ ID NO: 20)
MTVKYTDAQIQRLREYGNGTYEQKVFEDLASRDAAFSKEMSVAST
DNEKKIKGMIANPSRHGLTQLMNDIADALVAEGFIEVRTPIFISK
DALARMTITEDKPLFKQVFWIDEKRALRPMLAPNQYSVMRDLRDH
TDGPVKIFEMGSCFRKESHSGMHLEEFTMLSLVDMGPRGDATEVL
KNYISVVMKAAGLPDYDLVQEESDVYKETIDVEINGQEVCSAAVG
PHYLDAAHDVHEPWSGAGFGLERLLTIREKYSTVKKGGASISYLN
GAKINSG*

The following examples are provided for the purpose of illustrating, not limiting the disclosure.

### Examples

### General Synthetic Methods

All purchased chemicals were used without further purification. 3-Amino-PROXYL was purchased from Toronto Research Chemicals. Anhydrous dichloromethane (DCM) and dimethyl sulfoxide were used after overnight stirring with calcium hydride and distillation under argon atmosphere. Thin layer chromatography (TLC) was performed on silica 60F-254 plates. The TLC spots of alkene were identified by potassium permanganate staining. Flash chromatographic purification was performed using silica gel 60 (230-400 mesh size). ¹H NMR spectra were recorded on Bruker at 400 MHz and 700 MHz and ¹³C NMR spectra were recorded at 175 MHz. The chemical shifts were shown in ppm and are referenced to the residual non-deuterated solvent peak CDCl₃ (δ=7.26 in ¹H NMR, δ=77.23 in ¹³C NMR), CD₃OD (δ=3.31 in ¹H NMR, δ=49.2 in ¹³C NMR), d₆-DMSO (δ=2.5 in ¹H NMR, δ=39.5 in ¹³C NMR) as an internal standard. Splitting patterns of protons are designated as follows: s—singlet, d—doublet, t—triplet, q—quartet, m—multiplet, bs—broad singlet, dd—doublet of doublets.

### The Preparation and Characterization of Representative Tetrazine Amino Acids

The preparation and characterization of representative tetrazine amino acids (Tet-v4.0 amino acids: Tet-v4.0 methyl, Tet-v4.0 phenyl, Tet-v4.0 2-pyridinyl) is described below.

### (S)-2-amino-3-(6-methyl-1,2,4,5-tetrazin-3-yl)propanoic acid, trifluoro acetate salt (1a) (Tet-v4.0 methyl, trifluoro acetate salt)

Tet-v4.0 methyl was prepared as described below.

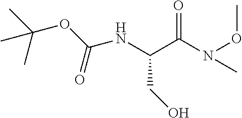 [SMILES: CON(C)C(=O)[C@H](CO)NC(=O)OC(C)(C)C]

Tert-butyl (S)-(3-hydroxy-1-(methoxy(methyl)amino)-1-oxopropan-2-yl) carbamate: To a solution of N-boc protected L-serine (2 gm, 9.7 mmol) in a dry DCM (50 mL) N, O dimethyl hydroxyl amine hydrochloride (1.4 gm, 10.7 mmol) was added, and the reaction mixture was stirred at −5° C. (ice and salt mixture) under nitrogen atmosphere. Then the solution was treated with N-methyl morpholine (1.2 mL, 10.7 mmol) and 1-ethyl-3-(3-dimethylaminopropyl) carbodiimide (2 gm, 10.7 mmol) was added in three portions over 30 minutes. After stirring at −5° C. for 2 hours the reaction mixture was treated with 1.0 M aqueous HCl solution and immediately extracted with 200 mL dichloromethane. The organic layer was washed with saturated sodium bicarbonate, then brine solution and dried over Na₂SO₄. The solvent was removed by rotary vacuum and afforded white solid compound utilized for next step without purification (yield—83%). ¹H NMR (CDCl₃; 400 MHz) δ 5.64 (bs, 1H), 4.8 (bs, 1H), 3.84-3.79 (m, 2H), 3.78 (s, 3H), 3.23 (s, 3H), 1.45 (s, 9H).

Tert-butyl (S)-(1-(methoxy(methyl)amino)-3-(methylsulfonyl)-1-oxopropan-2-yl)

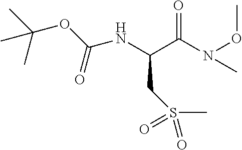 [SMILES: CON(C)C(=O)[C@@H](CS(C)(=O)=O)NC(=O)OC(C)(C)C]

carbamate (4): N,C-protected L-serine (1 gm, 4.1 mmol) was dissolved in a dry DCM under nitrogen atmosphere, the temperature was reduced to 0° C., then mesyl chloride (380 μL, 4.92 mmol) and triethyl amine (680 μL, 4.92 mmol) were added. The reaction mixture was stirred until the starting material was consumed as monitored by TLC (nearly an hour), then it was poured into water and DCM (50 ml each). The organic layer was washed with water two times, dried under sodium sulfate and then evaporated. The pure compound was isolated by silica gel flash column chromatography (40% ethyl acetate in hexanes) as brown oil (Yield—85%). ¹H NMR (CDCl₃; 400 MHz) δ 5.34 (d, 1H), 4.95 (bs, 1H), 4.43-4.49 (m, 2H), 3.79 (s, 3H), 3.04 (s, 3H), 3.26 (s, 3H), 1.46 (s, 9H).

(S)-2-((tert-butoxycarbonyl)amino)-3-cyanopropanoic acid (3): To a solution of the

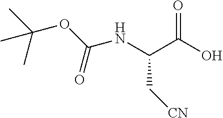

N,C protected mesylated serine (1 gm, 3.1 mmol) in dry DMSO (20 ml), sodium cyanide (380 mg, 7.8 mmol) was added and stirrer for 24 hrs. at 55° C. Next, 20 mL water was added to reaction mixture and extracted with ethyl acetate 3 times (20 mL each). The combined organic layer was washed with brine solution 3 times to remove the DMSO, then dried under anhydrous sodium sulfate and evaporated. The synthesized N,C protected beta cyano-alanine was isolated by silica gel flash column chromatography (hexane:ethyl acetate, 3:1), with a yield of 65%. Next, the C-terminal Weinreb amide was deprotected using 1.5 eqv. lithium hydroxide in (tetrahydrofuran) THF-water (10:1) system at 0° C. After completion of the reaction, the THF was removed by a rotary evaporator. Then 10 mL water was added to the reaction mixture and it was cooled to 0° C., then acidified (pH 3 to 4) with diluted acetic acid. The reaction mixture was extracted with DCM and the combined organic layers were dried over anhydrous sodium sulfate. The N-boc protected beta cyano-alanine was isolated by silica gel flash column chromatography (10% methanol in DCM) with a yield of 96%. ¹H NMR (700 MHz, CDCl₃) δ 5.77 (bs, 1H), 4.44 (bs, 1H), 2.99 (d, 1H), 2.91 (d, 1H); 1.41 (s, 9H). ¹³C NMR (175 MHz, CDCl₃—CD₃OD mix.) δ 171.1, 155.3, 116.8, 81.1, 50.1, 28.3, 21.8.

(S)-2-((tert-butoxycarbonyl)amino)-3-(6-methyl-1,2,4,5-tetrazin-3-yl)propanoic

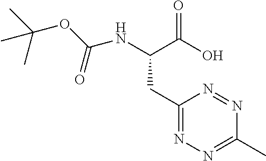 [SMILES: Cc1nnc(C[C@H](NC(=O)OC(C)(C)C)C(=O)O)nn1]

acid (2a): A flame dried, 15 mL heavy walled reaction tube was charged with boc-protected β-cyano L-alanine (200 mg, 0.934 mmol), nickel(II) trifluoromethanesulfonate (Ni(OTf)₂) (166 mg, 0.467 mmol) and acetonitrile (0.5 mL, 9.3 mmol) under argon atmosphere. Under argon anhydrous hydrazine (1.4 mL, 46.7 mmol) was added to the reaction mixture with stirring then the reaction vessel was purged with argon for 10 minutes. The sealed reaction mixture was heated to 35-37° C. with stirring for 32 hrs. The reaction mixture was cooled to room temperature, opened slowly, and 10 eqv. of aqueous 2 M sodium nitrite (NaNO₂) and 10 mL water were added with stirring. Next, the reaction mixture was washed with 20 mL ethyl acetate to remove the homocoupled product. The aqueous phase was acidified with 4 M HCl (about pH 2) under ice cold conditions then was extracted with ethyl acetate (3 times). The combined organic layer was dried with sodium sulfate and concentrated under reduced pressure. Silica gel flash column chromatography (30% ethyl acetate in hexanes with 1% acetic acid) of the resin yielded 143 mg of Boc-Tet4-Me (0.51 mmol, 54%) in the form of a pinkish red gummy material. ¹H NMR (400 MHz, CDCl₃) δ 5.62 (d, 1H), 4.54 (bs, 1H), 3.84 (dd, 2H), 3.07 (s, 3H), 1.47 (s, 9H). ¹³C NMR (175 MHz, CDCl₃) δ 172.1, 167.7, 166.3, 155.4, 81.2, 50.2, 37.1, 28.2, 21.6.

(S)-2-amino-3-(6-methyl-1,2,4,5-tetrazin-3-yl)propanoic acid trifluoro acetate salt

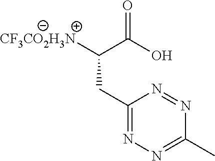

(1a): In a dry round-bottom flask, boc-protected Tet4-Me amino acid (120 mg, 0.423 mmol) was dissolved in 1:1 mixture of dry dichloromethane and trifluoroacetic acid (TFA) by volume (1.0 ml total) under argon. The reaction mixture was allowed to stir at room temperature for 1 hr. It was then concentrated under reduced pressure and dissolved in DCM to drive off TFA. This process was repeated twice prior to drying completely under high vacuum affording a pinkish red color TFA salt of Tet4-Me, with quantitative yield.

¹H NMR (700 MHz, CD₃OD) δ 4.48 (bs, 1H), 3.98 (dd, 1H), 3.83 (dd, 1H), 3.04 (s, 3H). ¹³C NMR (175 MHz, CD₃OD) δ 169.7, 167.7, 163.5, 51.9, 36.7, 21.3. ESI-MS calculated for C₆H₁₀N₅O₂([M+H]⁺) 184.0829, found 184.0834.

### (S)-2-amino-3-(6-phenyl-1,2,4,5-tetrazin-3-yl)propanoic acid, hydrochloride salt (1b) (Tet-v4.0 phenyl, hydrochloride salt)

Tet-v4.0 phenyl was prepared as described below.

(S)-2-((tert-butoxycarbonyl)amino)-3-(6-phenyl-1,2,4,5-tetrazin-3-yl)propanoic acid (2b): In a flame dried, 15 mL heavy walled reaction tube containing boc-protected β-cyano

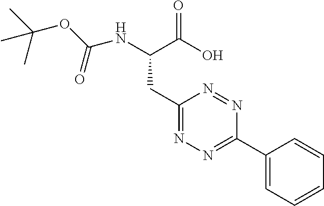 [SMILES: CC(C)(C)OC(=O)N[C@@H](Cc1nnc(-c2ccccc2)nn1)C(=O)O]

L-alanine (120 mg, 0.560 mmol) was charged with nickel(II) trifluoromethanesulfonate (Ni(OTf)₂) (98 mg, 0.28 mmol) and (benzonitrile (0.4 mL, 3.9 mmol) under an argon atmosphere. Then anhydrous hydrazine (1 mL, 28 mmol) and 0.3 mL ethanol (EtOH) were added to the reaction mixture and purged with argon for 10 minutes and the tube immediately sealed. The reaction mixture was heated to 42° C. for 30 hrs. The reaction mixture was cooled to room temperature, opened slowly and 10 eqv. 2 M sodium nitrite aqueous solution and 10 mL water were added to the reaction mixture. Then, the reaction mixture was washed with ethyl acetate to remove the homocoupled product. The aqueous phase was acidified with 4 M HCl (about pH 2) under ice cold conditions and subsequently extracted with ethyl acetate (3 times). The combined organic layer was dried with anhydrous sodium sulfate and concentrated under reduced pressure. Silica gel flash column chromatography (15% ethyl acetate in hexanes with 1% acetic acid) yielded 62 mg of Boc-Tet4-Ph (0.18 mmol, 32%) in the form of a reddish pink color gummy material. ¹H NMR (400 MHz, CDCl₃) δ 8.55 (d, 2H), 7.59-7.57 (m, 3H), 5.67 (d, 1H), 5.01 (bs, 1H), 3.96 (dd, 2H), 1.35 (s, 9H). ¹³C NMR (175 MHz, CDCl₃) δ 174.8, 166.7, 164.6, 155.6, 133.1, 131.6, 129.5, 128.3, 80.9, 51.9, 37.4, 28.4.

Hydrochloride salt (S)-2-amino-3-(6-phenyl-1,2,4,5-tetrazin-3-yl)propanoic acid (1b): In a dry round-bottom flask, boc-protected Tet4-Ph amino acid (80 mg, 0.23 mmol)

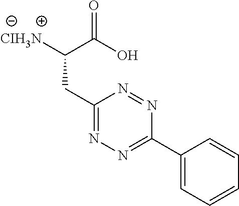

in 3 mL ethyl acetate was charged with 1 mL dioxane (saturated with N:N HCl gas) under an argon atmosphere. The reaction mixture was allowed to stir at room temperature for 2 hr. Then it was concentrated under reduced pressure, then dissolved in ethyl acetate. T his process was repeated 2 times to remove excess HCl gas. Finally, 5 mL pentane was added and dried completely under high vacuum resulting in a solid radish-pink color chloride salt of Tet4-Ph, with quantitative yield (˜97%). ¹H NMR (700 MHz, CD₃OD) δ 8.59 (d, 2H), 7.69 (t, 1H), 7.65 (t, 2H), 4.81 (dd, 1H), 4.07 (dd, 2H), 3.99 (dd, 2H). ¹³C NMR (175 MHz, CD₃OD) δ 170.6, 167.2, 166.3, 134.2, 133.4, 130.6, 129.2, 52.2, 36.22. ESI-MS calculated for C₁₁H₁₂N₅O₂ ([M+H]⁺) 246.0986, found 246.0994.

### (S)-2-amino-3-(6-(pyridin-2-yl)-1,2,4,5-tetrazin-3-yl)propanoic acid, hydrochloride salt (1c) (Tet-v4.0 pyridinyl, hydrochloride salt)

Tet-v4.0 pyridine was prepared as described below.

(S)-2-((tert-butoxycarbonyl)amino)-3-(6-(pyridin-2-yl)-1,2,4,5-tetrazin-3-yl)propanoic acid (2c): Following the synthetic procedure for 2b, the boc-protected β-cyano

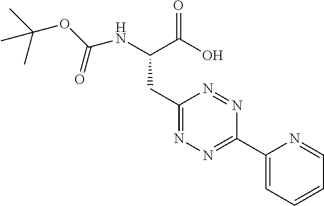 [SMILES: CC(C)(C)OC(=O)N[C@@H](Cc1nnc(-c2ccccn2)nn1)C(=O)O]

L-alanine (0.20 g, 0.93 mmol) and 2-pyridine carbonitrile (0.7 mL, 6.5 mmol) produced 0.145 g (0.42 mmol) of the title compound Boc-Tet4-Pyr as a pink color material with a yield 45%. ¹H NMR (400 MHz, CDCl₃) δ 8.96 (d, 1H), 8.68 (d, 1H), 8.05 (t, 1H), 7.62 (t, 1H), 5.71 (bs, 1H), 5.01 (bs, 1H), 4.03 (bs, 2H), 1.41 (s, 9H). ¹³C NMR (175 MHz, CDCl₃) δ 176.4, 174.1, 167.9, 155.6, 150.5, 149.8, 138.2, 127.1, 124.4, 80.5, 52.2, 37.7, 28.4.

Hydrochloride salt of (S)-2-amino-3-(6-(pyridin-2-yl)-1,2,4,5-tetrazin-3-yl)

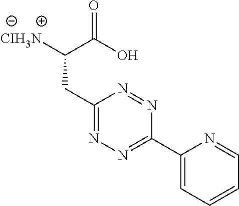

propanoic acid (1c): Following the boc-deprotection reaction for 1b, the chloride salt of Tet4-Pyr was generated in a near quantitative yield (97-98%). ¹H NMR (700 MHz, CD₃OD) δ 9.1 (d, 1H), 9.06 (d, 1H), 8.71 (t, 1H), 8.21 (t, 1H), 4.23 (dd, 1H), 4.13 (dd, 1H). ¹³C NMR (175 MHz, CD₃OD) δ 170.3, 169.1, 162.5, 147.6, 147.1, 146.3, 130.7, 127.2, 52.1, 36.5. ESI-MS calculated for C₁₀H₁₁N₆O₂ ([M+H]⁺) 247.0938, found 247.0932.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A tetrazine amino acid having formula (I): 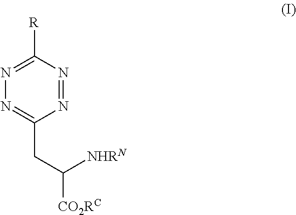
or a stereoisomer or salt thereof, wherein
R is selected from the group consisting of:
(a) a phenyl group substituted with a group selected from o-C1-C4 alkyl, m-C1-C4 alkyl, o-C1-C3 alkoxy, m-C1-C3 alkoxy, o-cyano, m-cyano, o-nitro, m-nitro, o- or m-primary amino (—NH₂), secondary amino (—NHR^{x}), or tertiary amino (—NR^{x}R^{y}) (wherein R^{x} and R^{y} are independently C1-C6 alkyl), o-fluoro, m-fluoro, 3,5-difluoro, 3,4,5-trifluoro, o-trifluoromethyl, m-trifluoromethyl, and o-, m-, or p-C(═O)R^{z} (wherein R^{z} a counterion, hydrogen, or C1-C6 alkyl),
(b) a substituted or an unsubstituted heteroaryl group,
(c) a substituted or an unsubstituted heterocyclyl group,
(d) an amino C1-C6 alkyl group,
(e) a thio C1-C6 alkyl group,
(f) a carboxylate group,
(g) a sulfonate group, and
(h) an amide group;
R^{C} is hydrogen, a counter ion, or a carboxyl protecting group; and
R^{N} is hydrogen or an amine protecting group.

2. The tetrazine amino acid of claim 1, wherein the C1-C6 alkyl group is selected from the group consisting of methyl, ethyl, n-propyl, i-propyl, n-butyl, s-butyl, t-butyl, n-pentyl, and n-hexyl.

3. The tetrazine amino acid of claim 1, wherein the phenyl group is substituted with a group selected from the group consisting of 3-methyl, 2-methoxy, 3-methoxy, 2-cyano, 3-cyano, 2-nitro, 3-nitro, 2-fluoro, 3-fluoro, 3,5-difluoro, 3,4,5-trifluoro, 2-hydroxy, 3-hydroxy, 2-amino, 3-amino, 2-acetyl, 3-acetyl, 2-trifluoromethyl, and 3-trifluoromethyl.

4. The tetrazine amino acid of claim 1, wherein the heteroaryl group is a pyridyl group, a furanyl group, a thiophenyl group, or an oxazolyl group.

5. The tetrazine amino acid of claim 1, wherein the heterocyclyl group is a pyrrolidinyl group, a piperadinyl group, a piperazinyl group, a tetrahydrofuranyl group, a tetrahydropyranyl, a tetrahydrothiophenyl group, an oxazolydinyl group, or a dihydropyran group.

6. The tetrazine amino acid of claim 1, wherein the amino C1-C6 alkyl group is an aminobutyl group.

7. The tetrazine amino acid of claim 1, wherein the thio C1-C6 alkyl group is a thiomethyl group.

8. The tetrazine amino acid of claim 1, wherein the carboxylate group is a C1-C6 alkyl carboxylate group.

9. The tetrazine amino acid of claim 1, wherein the amide group is a C1-C6 alkyl amide group.

10. A method for making a protein or a polypeptide of interest, comprising:
incorporating a tetrazine amino acid of claim 1, or a stereoisomer or salt thereof, into a protein or polypeptide.

11. A method for genetically encoding a protein or a polypeptide of interest, comprising:
incorporating a tetrazine amino acid of claim 1, or a stereoisomer or salt thereof, into a protein or polypeptide by genetic encoding.

12. A protein or polypeptide, comprising at least one tetrazine amino acid residue, wherein the tetrazine amino acid residue is derived from a tetrazine amino acid of claim 1, or a stereoisomer or salt thereof.

13. A protein or polypeptide, comprising at least one tetrazine amino acid residue, wherein the tetrazine amino acid residue is incorporated into the protein or polypeptide by genetic encoding of the protein or polypeptide using a tetrazine amino acid of claim 1, or a stereoisomer or salt thereof.

14. A composition comprising a protein or polypeptide,
wherein the protein or polypeptide comprises at least one tetrazine amino acid comprising a first reactive group and at least one post-translational modification,
wherein the tetrazine amino acid residue is derived from a tetrazine amino acid of claim 1, or a stereoisomer or salt thereof, and
wherein the at least one post-translational modification comprises attachment of a molecule comprising a second reactive group by a [4+2] cycloaddition reaction to the at least one tetrazine amino acid comprising the first reactive group.

15. A composition comprising a protein or polypeptide,
wherein the protein or polypeptide comprises at least one tetrazine amino acid comprising a first reactive group and at least one post-translational modification,
wherein the tetrazine amino acid residue is derived from genetic encoding of the protein or polypeptide using a tetrazine amino acid of claim 1, or a stereoisomer or salt thereof, and
wherein the at least one post-translational modification comprises attachment of a molecule comprising a second reactive group by a [4+2] cycloaddition reaction to the at least one tetrazine amino acid comprising the first reactive group.

16. A kit for in cellulo production of a tetrazine-labeled protein or a tetrazine-labeled polypeptide, comprising:
(a) a tRNA;
(b) an aminoacyl-tRNA synthetase; and
(c) a tetrazine amino acid of claim 1, or a stereoisomer or salt thereof,
wherein the tRNA and aminoacyl-tRNA synthetase are an orthogonal tRNA/orthogonal aminoacyl-tRNA pair effective for incorporating the compound into a protein or polypeptide to provide a tetrazine-labeled protein.


SEQUENCE LISTING (20 sequences)

SEQ ID NO: 1 - protein, 247 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 2 - DNA, 744 bp, synthetic construct
  source 1..744: mol_type = other DNA
atggttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataatgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccattaa

SEQ ID NO: 3 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKVFEVGPCYRKESDGKEHLEEFTMVSFGQMGSGCTRENLEALIKEFLDYLEIDFEIVGDSCMVYGDTLDIMHGDLELSSAHVGPVSLDREWGIDKPVIGAGFGLERLLKVMHGFKNIKRASRSESYYNGISTNL

SEQ ID NO: 4 - DNA, 1260 bp, synthetic construct
  source 1..1260: mol_type = other DNA
atggataaaaaaccgctggatgtgctgattagcgcgaccggcctgtggatgagccgtaccggcaccctgcataaaatcaaacatcatgaagtgagccgcagcaaaatctatattgaaatggcgtgcggcgatcatctggtggtgaacaacagccgtagctgccgtaccgcgcgtgcgtttcgtcatcataaataccgcaaaacctgcaaacgttgccgtgtgagcgatgaagatatcaacaactttctgacccgtagcaccgaaagcaaaaacagcgtgaaagtgcgtgtggtgagcgcgccgaaagtgaaaaaagcgatgccgaaaagcgtgagccgtgcgccgaaaccgctggaaaatagcgtgagcgcgaaagcgagcaccaacaccagccgtagcgttccgagcccggcgaaaagcaccccgaacagcagcgttccggcgtctgcgccggcaccgagcctgacccgcagccagctggatcgtgtggaagcgctgctgtctccggaagataaaattagcctgaacatggcgaaaccgtttcgtgaactggaaccggaactggtgacccgtcgtaaaaacgattttcagcgcctgtataccaacgatcgtgaagattatctgggcaaactggaacgtgatatcaccaaattttttgtggatcgcggctttctggaaattaaaagcccgattctgattccggcggaatatgtggaacgtatgggcattaacaacgacaccgaactgagcaaacaaattttccgcgtggataaaaacctgtgcctgcgtccgatgctggccccgaccctgtataactatctgcgtaaactggatcgtattctgccgggtccgatcaaagtttttgaagtgggcccgtgctatcgcaaagaaagcgatggcaaagaacacctggaagaattcaccatggtttcgtttggtcaaatgggcagcggctgcacccgtgaaaacctggaagcgctgatcaaagaattcctggattatctggaaatcgacttcgaaattgtgggcgatagctgcatggtgtatggcgataccctggatattatgcatggcgatctggaactgagcagcgcgcatgtgggtccggttagcctggatcgtgaatggggcattgataaaccggtgattggcgcgggttttggcctggaacgtctgctgaaagtgatgcatggcttcaaaaacattaaacgtgcgagccgtagcgaaagctactataacggcattagcacgaacctgtaa

SEQ ID NO: 5 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVSFAQMGSGCTRENLEALIKEFLDYLEIDFEIVGDSCMVYGDTLDIMHGDLELSSAHVGPVSLDREWGIDKPIIGAGFGLERLLKVMHGFKNIKRASRSESYYNGISTNL

SEQ ID NO: 6 - DNA, 1260 bp, synthetic construct
  source 1..1260: mol_type = other DNA
atggataaaaaaccgctggatgtgctgattagcgcgaccggcctgtggatgagccgtaccggcaccctgcataaaatcaaacatcatgaagtgagccgcagcaaaatctatattgaaatggcgtgcggcgatcatctggtggtgaacaacagccgtagctgccgtaccgcgcgtgcgtttcgtcatcataaataccgcaaaacctgcaaacgttgccgtgtgagcgatgaagatatcaacaactttctgacccgtagcaccgaaagcaaaaacagcgtgaaagtgcgtgtggtgagcgcgccgaaagtgaaaaaagcgatgccgaaaagcgtgagccgtgcgccgaaaccgctggaaaatagcgtgagcgcgaaagcgagcaccaacaccagccgtagcgttccgagcccggcgaaaagcaccccgaacagcagcgttccggcgtctgcgccggcaccgagcctgacccgcagccagctggatcgtgtggaagcgctgctgtctccggaagataaaattagcctgaacatggcgaaaccgtttcgtgaactggaaccggaactggtgacccgtcgtaaaaacgattttcagcgcctgtataccaacgatcgtgaagattatctgggcaaactggaacgtgatatcaccaaattttttgtggatcgcggctttctggaaattaaaagcccgattctgattccggcggaatatgtggaacgtatgggcattaacaacgacaccgaactgagcaaacaaattttccgcgtggataaaaacctgtgcctgcgtccgatgctggccccgaccctgtataactatctgcgtaaactggatcgtattctgccgggtccgatcaaaatttttgaagtgggcccgtgctatcgcaaagaaagcgatggcaaagaacacctggaagaattcaccatggtttcgtttgctcaaatgggcagcggctgcacccgtgaaaacctggaagcgctgatcaaagaattcctggattatctggaaatcgacttcgaaattgtgggcgatagctgcatggtgtatggcgataccctggatattatgcatggcgatctggaactgagcagcgcgcatgtgggtccggttagcctggatcgtgaatggggcattgataaaccgattattggcgcgggttttggcctggaacgtctgctgaaagtgatgcatggcttcaaaaacattaaacgtgcgagccgtagcgaaagctactataacggcattagcacgaacctgtaa

SEQ ID NO: 7 - protein, 398 aa, synthetic construct
MKHHHHHHPMSDYDIPTTENLYFQGAMAKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 8 - DNA, 1197 bp, synthetic construct
  source 1..1197: mol_type = other DNA
atgaaacatcaccatcaccatcaccccatgagcgattacgacatccccactactgagaatctttattttcagggcgccatggcgaaaactgaagaaggtaaactggtaatctggattaacggcgataaaggctataacggtctcgctgaagtcggtaagaaattcgagaaagatacaggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgttcacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgcatcaccaaataa

SEQ ID NO: 9 - DNA, 56 bp, synthetic construct
  source 1..56: mol_type = other DNA
gttttttgggctaacaggaggaattaacatgaaacatcaccatcaccatcacccca

SEQ ID NO: 10 - DNA, 57 bp, synthetic construct
  source 1..57: mol_type = other DNA
gagtttttgttcgggcncaagcttcgctcgagttattatttggtgatgcgagtctgc

SEQ ID NO: 11 - DNA, 72 bp, synthetic construct
  source 1..72: mol_type = other DNA
tggcggaaaccccgggaatctaacccggctgaacggatttagagtccattcgatctacatgatcaggttccc

SEQ ID NO: 12 - DNA, 75 bp, synthetic construct
  source 1..75: mol_type = other DNA
agatctgggggacggtccggcgaccagcgggtctctaaaacctagcatagcggggttcgacaccccggtctctcg

SEQ ID NO: 13 - DNA, 834 bp, synthetic construct
  source 1..834: mol_type = other DNA
atgacagtgaaatacacagatgcccagatccagcgcctgcgggagtatggcaacggcacctatgagcagaaggtgtttgaagatctggcctctagagatgcagccttctccaaggagatgtccgtggcttccacagacaacgagaaaaagatcaagggcatgattgccaaccccagccgccatgggctgacccagctgatgaatgacatcgccgacgccctggtggccgagggcttcatcgaggtcaggacccccatcttcatttctaaggacgcgctggctcgcatgaccatcaccgaggacaagcccctgttcaagcaggtgttctggatcgatgagaagagggctctgaggcccatgctcgcccccaaccagtactccgtgatgcgggacctgcgcgaccacacggacggccctgtgaaaattttcgaaatgggctcctgctttaggaaagaaagccacagcggaatgcacctggaggagttcaccatgctgagtctgtgtgacatggggccaagaggagatgccacagaagtgctgaagaactacatctcagtggtcatgaaggctgctggactgcccgactatgatttggtgcaggaagagagcgatgtctacaaagaaaccattgatgtggagatcaatggccaggaggtgtgctctgcttgtgtgggcccccactacctggacgccgcccacgacgtgcatgaaccctatagtggagcgggctttggcctggagaggctgctgaccataagagaaaagtacagcactgtgaagaaaggcggcgcctccatctcctacttgaatggagccaagatcaacagcggctga

SEQ ID NO: 14 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNQYSVMRDLRDHTDGPVKIFEMGSCFRKESHSGMHLEEFTMLSLCDMGPRGDATEVLKNYISVVMKAAGLPDYDLVQEESDVYKETIDVEINGQEVCSACVGPHYLDAAHDVHEPYSGAGFGLERLLTIREKYSTVKKGGASISYLNGAKINSG

SEQ ID NO: 15 - DNA, 75 bp, synthetic construct; identical to SEQ ID NO: 12
  source 1..75: mol_type = other DNA

SEQ ID NO: 16 - DNA, 834 bp, synthetic construct
  source 1..834: mol_type = other DNA
atgacagtgaaatacacagatgcccagatccagcgcctgcgggagtatggcaacggcacctatgagcagaaggtgtttgaagatctggcctctagagatgcagccttctccaaggagatgtccgtggcttccacagacaacgagaaaaagatcaagggcatgattgccaaccccagccgccatgggctgacccagctgatgaatgacatcgccgacgccctggtggccgagggcttcatcgaggtcaggacccccatcttcatttctaaggacgcgctggctcgcatgaccatcaccgaggacaagcccctgttcaagcaggtgttctggatcgatgagaagagggctctgaggcccatgctcgcccccaaccagtactccgtgatgcgggacctgcgcgaccacacggacggccctgtgaaaattttcgaaatgggctcctgctttaggaaagaaagccacagcggaatgcacctggaggagttcaccatgctgtctctggttgacatggggccaagaggagatgccacagaagtgctgaagaactacatctcagtggtcatgaaggctgctggactgcccgactatgatttggtgcaggaagagagcgatgtctacaaagaaaccattgatgtggagatcaatggccaggaggtgtgctctgctagtgtgggcccccactacctggacgccgcccacgacgtgcatgaaccctggagtggagcgggctttggcctggagaggctgctgaccataagagaaaagtacagcactgtgaagaaaggcggcgcctccatctcctacttgaatggagccaagatcaacagcggctga

SEQ ID NO: 17 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNQYSVMRDLRDHTDGPVKIFEMGSCFRKESHSGMHLEEFTMLSLVDMGPRGDATEVLKNYISVVMKAAGLPDYDLVQEESDVYKETIDVEINGQEVCSASVGPHYLDAAHDVHEPWSGAGFGLERLLTIREKYSTVKKGGASISYLNGAKINSG

SEQ ID NO: 18 - DNA, 75 bp, synthetic construct; identical to SEQ ID NO: 12
  source 1..75: mol_type = other DNA

SEQ ID NO: 19 - DNA, 834 bp, synthetic construct
  source 1..834: mol_type = other DNA
atgacagtgaaatacacagatgcccagatccagcgcctgcgggagtatggcaacggcacctatgagcagaaggtgtttgaagatctggcctctagagatgcagccttctccaaggagatgtccgtggcttccacagacaacgagaaaaagatcaagggcatgattgccaaccccagccgccatgggctgacccagctgatgaatgacatcgccgacgccctggtggccgagggcttcatcgaggtcaggacccccatcttcatttctaaggacgcgctggctcgcatgaccatcaccgaggacaagcccctgttcaagcaggtgttctggatcgatgagaagagggctctgaggcccatgctcgcccccaaccagtactccgtgatgcgggacctgcgcgaccacacggacggccctgtgaaaattttcgaaatgggctcctgctttaggaaagaaagccacagcggaatgcacctggaggagttcaccatgctgagtctggtggacatggggccaagaggagatgccacagaagtgctgaagaactacatctcagtggtcatgaaggctgctggactgcccgactatgatttggtgcaggaagagagcgatgtctacaaagaaaccattgatgtggagatcaatggccaggaggtgtgctctgctgccgtgggcccccactacctggacgccgcccacgacgtgcatgaaccctggagtggagcgggctttggcctggagaggctgctgaccataagagaaaagtacagcactgtgaagaaaggcggcgcctccatctcctacttgaatggagccaagatcaacagcggctga

SEQ ID NO: 20 - protein, 277 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNQYSVMRDLRDHTDGPVKIFEMGSCFRKESHSGMHLEEFTMLSLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKINSG